(19) United States
(12) Patent Application Publication
(10) Pub. No.: US 2019/0269331 A1
(43) Pub. Date: Sep. 5, 2019

(54) ULTRASOUND MODULATING OPTICAL TOMOGRAPHY USING REDUCED LASER PULSE DURATION

(71) Applicant: HI LLC, Los Angeles, CA (US)

(72) Inventors: Jamu Alford, Simi Valley, CA (US); Adam Marblestone, Arlington, MA (US); Changhuei Yang, Pasadena, CA (US)

(73) Assignee: HI LLC, Los Angeles, CA (US)

(21) Appl. No.: 16/266,818

(22) Filed: Feb. 4, 2019

Related U.S. Application Data
(60) Provisional application No. 62/637,703, filed on Mar. 2, 2018.

(51) Int. Cl. A61B 5/00; G01B 9/02

(52) U.S. Cl. CPC A61B 5/0066; A61B 8/4416; G01B 9/02091; A61B 5/0035

(57) ABSTRACT

Sample light is delivered into the anatomical structure having a target voxel, whereby a portion of the sample light passing through the target voxel is scattered by the anatomical structure as signal light, and another portion of the sample light not passing through the target voxel is scattered by the anatomical structure as background light that is combined with the signal light to create a sample light pattern. Reference light is combined with the sample light pattern to generate an interference light pattern, such that the signal light and the reference light are combined in a heterodyne manner. Ultrasound is delivered into the target voxel, such that the signal light is frequency shifted by the ultrasound. The ultrasound and the sample light are pulsed in synchrony at the target voxel, such that at least one pulse of the sample light has a combined duration less than a pulse width of the ultrasound.

### RELATED APPLICATION DATA

This application claims the benefit of U.S. Provisional Patent Application 62/637,703, filed Mar. 2, 2018, which is expressly incorporated herein by reference.

### FIELD OF THE INVENTION

The present invention relates to methods and systems for non-invasive measurements in the human body, and in particular, methods and systems related to detecting physiologically dependent optical parameters in the human body, animal body, and/or biological tissue.

### BACKGROUND OF THE INVENTION

Measuring neural activity in the brain is useful for medical diagnostics, neuromodulation therapies, neuroengineering, or brain-computer interfacing. For example, it may be desirable to measure neural activity in the brain of a patient to determine if a particular region of the brain has been impacted by reduced blood irrigation, a hemorrhage, any other type of damage. For instance, in cases where the patient has suffered a traumatic brain injury, such as stroke, it may be desirable to determine whether the patient should undergo a therapeutic procedure. Measuring neural activity in the brain also may be used to determine the efficacy of such a therapeutic procedure.

Conventional methods for measuring neural activity in the brain include diffuse optical tomography (DOT), and functional near-infrared spectroscopy (fNIRS), as well as others. These applications only employ a moderate amount of near-infrared or visible light radiation, thus being comparatively safe and gentle for a biological subject in comparison to X-Ray Computed Tomography (CT) scans, positron emission tomography (PET), or other methods that use higher-energy and potentially harmful radiation. Moreover, in contrast to methods, such as functional magnetic resonance imaging (fMRI), these optically-based imaging methods do not require large magnets or magnetic shielding, and thus, can be scaled to wearable or portable form factors, which is especially important in applications such as brain-computer interfacing.

Because DOT and fNIRS rely on light, which scatters many times inside brain, skull, dura, pia, and skin tissues, the light paths occurring in these techniques comprise random or “diffusive” walks, and therefore, only limited spatial resolution can be obtained by a conventional optical detector, often on the order of centimeters. The reason for this limited spatial resolution is that the paths of photons striking the detector in such schemes are highly variable and difficult, and even impossible to predict without detailed microscopic knowledge of the scattering characteristics of the brain volume of interest, which is typically unavailable in practice (i.e., in the setting of non-invasive measurements through skull for brain imaging and brain interfacing). In summary, light scattering prevents optical imaging from achieving high resolution deep inside tissue.

There is an increasing interest in ultrasound modulated optical tomography (UOT) to detect more precisely localized changes in biological tissues, e.g., on a sub-millimeter length scale, inside thick biological tissue, such as the brain (see U.S. Pat. No. 8,423,116; Sakadzic S, Wang L V, “High-Resolution Ultrasound-Modulated Optical Tomography in Biological Tissues,” Optics Letters, Vol. 29, No. 23, pp. 2770-2772, Dec. 1, 2004). These localized changes may include changes in light absorption in the brain that reflect neural activity and neurovascular coupling, such as a blood-oxygen-level dependent signal, for application in diagnostics, therapeutics, or, notably, brain computer interfacing (see Steinbrink J, Villringer A, Kempf F, Haux D. Boden S, Obrig H., “Illuminating the BOLD Signal: Combined fMRI-fNIRS Studies,” Magnetic Resonance Imaging, Vol. 24, No. 4, pp. 495-505, May 31, 2006). Thus, there is an increasing interest in ultrasound modulated optical tomography (UOT) in biomedical applications due to its potential to simultaneously achieve good resolution and imaging depth.

In UOT, a highly localized ultrasound focus, e.g., millimeter or sub-millimeter in size, is used to selectively perturb (i.e., “tag”) light (e.g., light generated by a near-infrared coherent laser) passing through a voxel size of tissue defined by the size of the ultrasound focus. Due to the acousto-optic effect, light passing through the ultrasonic beam undergoes a frequency shift defined by multiples of the ultrasonic frequency. By detecting the frequency-shifted light, i.e., the tagged light, spatial information characterizing the biological tissue within the voxel can be acquired. As a result, spatial resolution is boosted from the centimeter-scale diffusive spread of light in the biological tissue to approximately a millimeter-scale voxel size. This ultrasound tagging of light relies on mechanisms known in the field (see Mahan G D, Engler W E, Tiemann J J, Uzgiris E, “Ultrasonic Tagging of Light: Theory,” Proceedings of the National Academy of Sciences, Vol. 95, No. 24, pp. 14015-14019, Nov. 24, 1998).

Typical UOT implementations generate weak signals that make it difficult to differentiate ultrasound-tagged light passing through the focal voxel from a much larger amount of unmodulated light which is measured as DC shot noise. Thus, conventional UOT has the challenge of obtaining optical information through several centimeters of biological tissue, for example, noninvasive measurements through the human skull used to measure functional changes in the brain.

Various methods have been developed to detect the very small fraction of tagged light out of a large background of untagged light by detecting the speckle pattern of light resulting from the interference of many multiply-scattered optical waves with different phases and amplitudes, which combine in a resultant wave whose amplitude, and therefore intensity, as well as phase, varies randomly. In the context of neuroengineering and brain computer interfacing, a key challenge is to render these methods to be sufficiently sensitive to be useful for through-human-skull functional neuroimaging.

One technique uses a narrow spectral filter to separate out the untagged light striking a single-pixel detector, and is immune to “speckle decorrelation” (greater than ˜0.1 ms-1 ms) due to the scatterers' motion (for example, blood flow) inside living biological tissue, but requires bulky and expensive equipment.

Another technique uses crystal-based holography to combine a reference light beam and the sample light beam into a constructive interference pattern, but can be adversely affected by rapid speckle decorrelation, since the response time of the crystal is usually much longer than the speckle correlation time.

Still another technique, referred to as heterodyne parallel speckle detection (PSD), employs optical interference together with a spatially resolved detector array (e.g., a conventional charge-coupled device (CCD) camera) used as an array of independent detectors for collecting the signal over a large number of coherence areas (see Atlan M, Forget B C, Ramaz F, Boccara A C, Gross M, “Pulsed Acousto-Optic Imaging in Dynamic Scattering Media With Heterodyne Parallel Speckle Detection,” Optics Letter, Vol. 30, No. 11, pp. 1360-1362, Jun. 1, 2005). Such configuration improves the signal-to-noise ratio relative to a single-detector and relative to approaches based on other modes of separating tagged and untagged light, such as spectral filters. However, the conventional CCD cameras used for heterodyne PSD have low frame rates, and therefore suffer from a relatively low speed relative to the speckle decorrelation time, thereby making this set up insufficient for in vivo deep tissue applications. Furthermore, conventional CCD cameras record both the AC signal and the DC background for each pixel. Thus, only a few bits of a pixel value can be used to represent the useful AC signal, while most of the bits are wasted in representing the DC background, resulting in a low efficiency in the use of bits.

Besides the challenges posed by the signal-to-noise ratio, speckle decorrelation time, and efficient pixel bit processing, another challenge involves obtaining sufficient axial resolution (i.e., the depth resolution or ultrasound propagation direction). To address this challenge, UOT has been applied in a pulsed wave (PW) mode for heterodyne PSD, rather than a continuous wave (CW) mode (see Li Y Zhang H, Kim C, Wagner K H, Hemmer P., Wang L V, “Pulsed Ultrasound-Modulated Optical Tomography Using Spectral-Hole Burning as a Narrowband Spectral Filter,” Applied Physics Letters, Vol. 93, No. 1, 011111, Jul. 7, 2008; Ruan H, Mather M L, Morgan S P, “Pulsed Ultrasound Modulated Optical Tomography with Harmonic Lock-In Holography Detection,” JOSA A, Vol. 30, No. 7, pp. 1409-1416, Jul. 1, 2013).

PW UOT has the benefit of enabling improved axial resolution compared to CW UOT. That is, with CW UOT, any light passing through the tissue, even though outside of the focal voxel, may be inadvertently tagged by the continuously propagating ultrasound energy along the ultrasound axis, thereby decreasing the signal-to-noise ratio. With PW UOT, the light passing through the tissue is pulsed only when the ultrasound pulses travels through the focal voxel, such that light outside of the focal voxel will not be tagged by the ultrasound energy. Although PW UOT improves axial resolution, the pulsed UOT signals are weak relative to continuous UOT signals.

Lock-in cameras, as compared to conventional CCD cameras, have been used to demodulate frequency-encoded light signals, e.g., to selectively extract ultrasound-modulated light from a light field consisting of a combination of ultrasound-modulated and unmodulated light, which has been theorized in ultrasound modulated optical tomography (UOT) (see Liu Y, Shen Y, Ma C, Shi J, Wang L V, “Lock-in Camera Based Heterodyne Holography for Ultrasound-Modulated Optical Tomography Inside Dynamic Scattering Media,” Applied Physics Letters, Vol. 108, No. 23, 231106, Jun. 6, 2016), and digital optical phase conjugation (DOPC) (see Liu Y, Ma C, Shen Y, Wang L V, “Bit-Efficient, Sub-Millisecond Wavefront Measurement Using a Lock-In Camera for Time-Reversal Based Optical Focusing Inside Scattering Media,” Optics Letters, Vol. 41, No. 7, pp. 1321-1324, Apr. 1, 2016).

A “lock-in camera” can be defined as a form of digital camera that can rapidly sample and store changes in a light field, in precise temporal synchrony with an external trigger signal, and which may also perform on-chip computations on the stored samples. The key feature of lock-in cameras is the ability to rapidly capture and store multiple sequential samples of the light field, which sample-to-sample latencies shorter than readout times of conventional cameras. Essentially, lock-in cameras capture and store, at each pixel multiple captured intensity values taken in short succession, with each captured intensity value stored in a separate data storing “bin,” rather than storing only a single captured intensity value as in conventional cameras. This enables lock-in cameras, for example, to sample a modulated light field at the same frequency as the modulation, such that subtraction across successive samples, or other operations, such as quadrature detection, will extract the component of the light that is modulated at the modulation frequency, while subtracting off the unmodulated (“DC”) background. Similarly, lock-in cameras can be used to make a series of such measurements or comparisons, locked to an external trigger signal, rapidly in order to extract such modulated components from a rapidly changing light field arising from dynamic, disordered biological specimen.

The use of lock-in cameras for measurement and demodulation of modulated light fields has, however, a number of disadvantages, particularly in the context of rapid measurement signals from dynamic, strongly scattering biological tissues. First, lock-in cameras cannot sample a light field arbitrarily fast, and therefore, have a minimum latency between the data storing bins. Second, lock-in cameras have only achieved limited scale to date, e.g., less than 100,000 pixels (e.g., the Heliotis Helicam C3), and do not have the large industrial support base of the conventional camera industry (e.g., with a digital camera that is now included within every smart phone). Third, lock-in cameras support only a limited number of data storing “bins” per pixel (currently, four bins per pixel) due to limitations on pixel area and photodetector fill factor, and thus, support only a limited number of temporal samples in the lock-in detection process.

The first of these disadvantages, in particular the limited sampling speed, poses a key challenge for the use of lock-in cameras to support imaging deep inside dynamic, highly scattering biological tissues, such as the human skull and brain. In particular, in the setting of UOT of a dynamic, highly scattering biological tissue using lock-in detection, a series of multiple lock-in camera detection events (bins) must be acquired within the “speckle decorrelation time” of the biological medium, which rapidly decreases with the depth at which the tissue is to be imaged, falling to microseconds or below as the imaging depth extends to the multi-centimeter range, and scaling inversely as the square of the imaging depth into tissue. This poses an obstacle for lock-in camera based detection, since the speed of lock-in cameras is limited.

In the context of PW UOT, it is generally desirable, within the constraints of a sufficient axial resolution, to maximize the width of the optical pulses generated by the pulsed lasers, such that the efficiency of the PW UOT detection scheme is maximized. However, for example, in some applications with specific requirements in power and wavelength, the maximum width of optical pulses generated by readily available pulsed lasers is limited; for example, the maximum pulse width of conventional off-the-shelf pulsed lasers is currently 200 ns, thereby requiring a pulsed laser to be customized, and increasing the cost and complexity of the UOT system.

Thus, although the UOT schemes described above may be sufficient for certain applications, particularly when using lock-in cameras, such UOT schemes are inappropriate for the application of 3D-resolved, highly sensitive detection of small signals (e.g., blood-oxygen-level dependent signals) non-invasively through thick scattering layers, such as the human skull.

### SUMMARY OF THE INVENTION

In accordance with one aspect of the present inventions, a pulsed ultrasound modulated optical tomography (UOT) system comprises an interferometer configured for delivering sample light into the anatomical structure having a target voxel (e.g., one less than one mm³), whereby a portion of the sample light passing through the target voxel is scattered by the anatomical structure as signal light, and another portion of the sample light not passing through the target voxel is scattered by the anatomical structure as background light that is combined with the signal light to create a sample light pattern. In one embodiment, the interferometer comprises a light source configured for generating source light, a beam splitter configured for splitting the source light into the sample light and the reference light. The interferometer is further configured for combining reference light with the sample light pattern to generate at least one interference light pattern (e.g., a speckle light pattern), such that at least one component of the signal light and the reference light are combined in a heterodyne manner.

The pulsed UOT system further comprises an acoustic assembly configured for delivering ultrasound into the target voxel, such that the signal light is frequency shifted by the ultrasound, and a controller configured for operating the acoustic assembly and the interferometer to pulse the ultrasound and the sample light in synchrony at the target voxel, such that at least one pulse (e.g., a single pulse or multiple pulses) of the sample light has a combined duration less than a pulse width of the ultrasound (e.g., a combined duration less than 500 ns, or even less than 200 ns).

In one embodiment, the interferometer is further configured for shifting the sample light relative to the reference light by a frequency different from the frequency of the ultrasound, such that the signal light and the reference light are combined in the heterodyne manner, e.g., such that

$${\int }_{0}^{1 / {f}_{shift}} \sqrt{{P}_{reference} ( t ) * {P}_{background} ( t )} \times sin ( 2 π {f}_{shift} + {α}_{unknown} ) dt \approx 0 ,$$

wherein P_{reference}(t) is the time varying component of the reference light as a function of time t, P_{background}(t) is the time varying component of the background light as a function of time t, and f_{shift} equals the frequency at which the sample light is shifted relative to the reference light by the interferometer. At least 99% of P_{background}(t) may be eliminated from the interference light pattern. In one embodiment, the pulse(s) of the sample light is a single rectangular pulse, and f_{shift}*Tₒₚ=1, wherein Tₒₚ is the width of the single rectangular pulse. In another embodiment, the pulse(s) of the sample light comprises two identical pulses (e.g., Gaussian pulses or even arbitrarily-shaped pulses) separated from each other by 1/(2*f_{shift}).

The pulsed UOT system further comprises at least one array of detectors configured for detecting spatial components of each of the at least one interference light pattern and storing a plurality of values representative of the respective spatial components of each of the at least one interference light pattern. The array(s) of detectors may be configured for detecting the spatial components of the different interference light pattern(s), and storing the plurality of values for all of the at least one interference pattern within, e.g., 10 milliseconds, and even, within 1 millisecond.

The pulsed UOT system further comprises a processor configured for determining a physiologically-dependent optical parameter of the target voxel based on the plurality of values. In one embodiment, the target voxel may comprise brain matter, in which case, the processor may be configured for determining neural activity within the target voxel based on the determined physiologically-dependent optical parameter. In one embodiment, the processor is configured for computing the amplitude of the signal light using the plurality of values generated by each detector array, and determining the physiologically-dependent optical parameter of the target voxel based on the computed amplitude of the signal light. If the plurality of values generated by each detector array are intensities of the spatial components of the respective interference light pattern, the processor may be configured for using the plurality of values generated by each detector array to determine a product of the amplitude of the signal light and a known amplitude of the reference light, and determining the amplitude of the signal light from the determined product.

In accordance with a second aspect of the present inventions, a method of performing pulsed ultrasound modulated optical tomography (UOT) comprises delivering ultrasound into a target voxel (e.g., one less than one mm³) within an anatomical structure, and delivering sample light into the anatomical structure, whereby a portion of the sample light passing through the target voxel is scattered by the anatomical structure as signal light, and another portion of the sample light not passing through the target voxel is scattered by the anatomical structure as background light that combines with the signal light to create a sample light pattern. The method may further comprise generating source light, and splitting the source light into the sample light and the reference light.

The method further comprises pulsing the ultrasound and the sample light in synchrony at target voxel, such that only the signal light is frequency shifted by the ultrasound, such that at least one pulse of the sample light has a combined duration less than a pulse width of the ultrasound (e.g., a single pulse or multiple pulses) of the sample light has a combined duration less than a pulse width of the ultrasound (e.g., a combined duration less than 500 ns, or even less than 200 ns), and combining reference light with the sample light pattern to generate at least one interference light pattern (e.g., a speckle light pattern), such that at least one component of the signal light and the reference light are combined in a heterodyne manner.

One method further comprises shifting the sample light relative to the reference light by a frequency different from the frequency of the ultrasound, such that the signal light and the reference light are combined in the heterodyne manner, e.g., such that

$${\int }_{0}^{1 / {f}_{shift}} \sqrt{{P}_{reference} ( t ) * {P}_{background} ( t )} \times sin ( 2 π {f}_{shift} + {α}_{unknown} ) dt \approx 0 ,$$

wherein P_{reference}(t) is the time varying component of the reference light 42 as a function of time t, P_{background}(t) is the time varying component of the background light 46 as a function of time t, and f_{shift} equals the frequency at which the sample light is shifted relative to the reference light by the interferometer. At least 99% of P_{background}(t) may be eliminated from the interference light pattern. In one embodiment, the pulse(s) of the sample light is a single rectangular pulse, and f_{shift}*Tₒₚ=1, wherein Tₒₚ is the width of the single rectangular pulse. In another embodiment, the pulse(s) of the sample light comprises two identical pulses (e.g., Gaussian pulses or even arbitrarily-shaped pulses) separated from each other by 1/(2*f_{shift}).

The method further comprises detecting spatial components of each of the interference light pattern(s), and storing a plurality of values representative of the respective spatial components of each of the at least one interference light pattern, e.g., 10 milliseconds, and even, within 1 millisecond.

The method further comprises determining a physiologically-dependent optical parameter of the target voxel based on the plurality of values. In one method, the target voxel comprises brain matter, in which case, the method may further comprise determining neural activity within the target voxel based on the determined physiologically-dependent optical parameter.

One method comprises computing the amplitude of the signal light using the plurality of values generated by each detector array, and determining the physiologically-dependent optical parameter of the target voxel based on the computed amplitude of the signal light. If the plurality of values generated by each detector array are intensities of the spatial components of the respective interference light pattern, the plurality of values generated by each detector array may be used to determine a product of the amplitude of the signal light and a known amplitude of the reference light, and determining the amplitude of the signal light from the determined product.

In accordance with a third aspect of the present inventions, a pulsed ultrasound modulated optical tomography (UOT) system comprises an interferometer configured for delivering sample light into the anatomical structure having a target voxel (e.g., one less than one mm³), whereby a portion of the sample light passing through the target voxel is scattered by the anatomical structure as signal light, and another portion of the sample light not passing through the target voxel is scattered by the anatomical structure as background light that is combined with the signal light to create a sample light pattern. In one embodiment, the interferometer comprises a light source configured for generating source light, a beam splitter configured for splitting the source light into the sample light and the reference light. The interferometer is further configured for combining reference light with the sample light pattern to generate at least one interference light pattern (e.g., a speckle light pattern), such that at least one component of the signal light and the reference light are combined in a heterodyne manner.

The pulsed UOT system further comprises an acoustic assembly configured for delivering ultrasound into the target voxel, such that the signal light is frequency shifted by the ultrasound, and a controller configured for operating the acoustic assembly and the interferometer to pulse the ultrasound and the sample light in synchrony at the target voxel, such that at least one pulse (e.g., a single pulse or multiple pulses) of the sample light has a combined duration different from a pulse width of the ultrasound (e.g., a combined duration less than 500 ns, or even less than 200 ns).

The interferometer is further configured for shifting the sample light relative to the reference light by a frequency different from the frequency of the ultrasound, such that the signal light and the reference light are combined in the heterodyne manner, e.g., such that

$${\int }_{0}^{1 / {f}_{shift}} \sqrt{{P}_{reference} ( t ) * {P}_{background} ( t )} \times sin ( 2 π {f}_{shift} + {α}_{unknown} ) dt \approx 0 ,$$

wherein P_{reference}(t) is the time varying component of the reference light 42 as a function of time t, P_{background}(t) is the time varying component of the background light as a function of time t, and f_{shift} equals the frequency at which the sample light is shifted relative to the reference light by the interferometer. At least 99% of the time varying component of P_{background}(t) may be eliminated from the interference light pattern. In one embodiment, the pulse(s) of the sample light is a single rectangular pulse, and f_{shift}*Tₒₚ=1, wherein Tₒₚ is the width of the single rectangular pulse. In another embodiment, the pulse(s) of the sample light comprises two identical pulses (e.g., Gaussian pulses or even arbitrarily-shaped pulses) separated from each other by 1/(2*f_{shift}).

The pulsed UOT system further comprises at least one array of detectors configured for detecting spatial components of each of the at least one interference light pattern and storing a plurality of values representative of the respective spatial components of each of the at least one interference light pattern. The array(s) of detectors may be configured for detecting the spatial components of the different interference light pattern(s), and storing the plurality of values for all of the at least one interference pattern within, e.g., 10 milliseconds, and even, within 1 millisecond.

The pulsed UOT system further comprises a processor configured for determining a physiologically-dependent optical parameter of the target voxel based on the plurality of values. In one embodiment, the target voxel may comprise brain matter, in which case, the processor may be configured for determining neural activity within the target voxel based on the determined physiologically-dependent optical parameter. In one embodiment, the processor is configured for computing the amplitude of the signal light using the plurality of values generated by each detector array, and determining the physiologically-dependent optical parameter of the target voxel based on the computed amplitude of the signal light. If the plurality of values generated by each detector array are intensities of the spatial components of the respective interference light pattern, the processor may be configured for using the plurality of values generated by each detector array to determine a product of the amplitude of the signal light and a known amplitude of the reference light, and determining the amplitude of the signal light from the determined product.

In accordance with a fourth aspect of the present inventions, a method of performing pulsed ultrasound modulated optical tomography (UOT) comprises delivering ultrasound into a target voxel (e.g., one less than one mm³) within an anatomical structure, and delivering sample light into the anatomical structure, whereby a portion of the sample light passing through the target voxel is scattered by the anatomical structure as signal light, and another portion of the sample light not passing through the target voxel is scattered by the anatomical structure as background light that combines with the signal light to create a sample light pattern. The method may further comprise generating source light, and splitting the source light into the sample light and the reference light.

The method further comprises pulsing the ultrasound and the sample light in synchrony at the target voxel, such that only the signal light is frequency shifted by the ultrasound, such that at least one pulse of the sample light has a combined duration different from a pulse width of the ultrasound (e.g., a single pulse or multiple pulses) of the sample light has a combined duration less than a pulse width of the ultrasound (e.g., a combined duration less than 500 ns, or even less than 200 ns), and combining reference light with the sample light pattern to generate at least one interference light pattern (e.g., a speckle light pattern), such that at least one component of the signal light and the reference light are combined in a heterodyne manner.

The method further comprises shifting the sample light relative to the reference light by a frequency different from the frequency of the ultrasound, such that the signal light and the reference light are combined in the heterodyne manner, e.g., such that

$${\int }_{0}^{1 / {f}_{shift}} \sqrt{{P}_{reference} ( t ) * {P}_{background} ( t )} \times sin ( 2 π {f}_{shift} + {α}_{unknown} ) dt \approx 0 ,$$

wherein P_{reference}(t) is a time varying component of the reference light as a function of time t, P_{background}(t) is a time varying component of the background light as a function of time t, and f_{shift} equals the frequency at which the sample light is shifted relative to the reference light by the interferometer. At least 99% of the time varying component of the background light P_{background}(t) may be eliminated from the interference light pattern. In one embodiment, the pulse(s) of the sample light is a single rectangular pulse, and f_{shift}*Tₒₚ=1, wherein Tₒₚ is the width of the single rectangular pulse. In another embodiment, the pulse(s) of the sample light comprises two identical pulses (e.g., Gaussian pulses or even arbitrarily-shaped pulses) separated from each other by 1/(2*f_{shift}).

The method further comprises detecting spatial components of each of the interference light pattern(s), and storing a plurality of values representative of the respective spatial components of each of the at least one interference light pattern, e.g., 10 milliseconds, and even, within 1 millisecond.

The method further comprises determining a physiologically-dependent optical parameter of the target voxel based on the plurality of values. In one method, the target voxel comprises brain matter, in which case, the method may further comprise determining neural activity within the target voxel based on the determined physiologically-dependent optical parameter.

One method comprises computing the amplitude of the signal light using the plurality of values generated by each detector array, and determining the physiologically-dependent optical parameter of the target voxel based on the computed amplitude of the signal light. If the plurality of values generated by each detector array are intensities of the spatial components of the respective interference light pattern, the plurality of values generated by each detector array may be used to determine a product of the amplitude of the signal light and a known amplitude of the reference light, and determining the amplitude of the signal light from the determined product.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a block diagram of an ultrasound modulating optical tomography (UOT) system constructed in accordance with one embodiment of the present inventions;

FIG. 2 a block diagram of one embodiment of an acoustic assembly used in the UOT system of FIG. 1;

FIG. 3a is a block diagram of one embodiment of an interferometer used in the UOT system of FIG. 1;

FIG. 3b is a block diagram of another embodiment of an interferometer used in the UOT system of FIG. 1;

FIG. 3c is a block diagram of still another embodiment of an interferometer used in the UOT system of FIG. 1;

FIG. 3d is a block diagram of yet another embodiment of an interferometer used in the UOT system of FIG. 1;

FIG. 4 is a schematic diagram of one embodiment of a detector array used in the UOT system of FIG. 1;

FIG. 5 is a timing diagram of one pulsing sequence used by the UOT system of FIG. 1 to detect a physiologically-dependent optical parameter in a target voxel within an anatomical structure;

FIG. 6 is a schematic diagram of the UOT system of FIG. 1, particularly showing the generation of interference light patterns, the detection of spatial components, and binning of spatial component values;

FIG. 7 is a timing diagram of another pulsing sequence used by the UOT system to detect a physiologically-dependent optical parameter in a target voxel within an anatomical structure;

FIG. 8 is a plan diagram of one embodiment of an optical assembly used to split a single optical pulse into a train of identical optical pulses for use in the UOT system of FIG. 1;

FIG. 9a is a schematic diagram of a modified UOT system of FIG. 1, particularly showing a detection of a wavefront of signal light;

FIG. 9b is a schematic diagram of a modified UOT system of FIG. 1, particularly showing playback of a phase conjugate of the wavefront of signal light;

FIG. 10a is a block diagram of one embodiment of a phase conjugation array that can be incorporated into the UOT system of FIG. 1, particularly showing detection of the signal light;

FIG. 10b is a block diagram of the phase conjugation array of FIG. 10a, particularly showing playback of a phase conjugation light field;

FIG. 11 is a plot of the absorption of light in water over the wavelength of light;

FIG. 12 is a plan view of wearable and unwearable units in which the UOT system of FIG. 1 may be embodied;

FIG. 13 is a flow diagram of one method used by the UOT system of FIG. 1 to non-invasively measure a physiologically-dependent optical parameter using the pulse sequence of FIG. 5;

FIG. 14 is a flow diagram of another method used by the UOT system of FIG. 1 to non-invasively measure a physiologically-dependent optical parameter using the pulse sequence of FIG. 7;

FIG. 15 is a block diagram of an ultrasound modulating optical tomography (UOT) system constructed in accordance with another embodiment of the present inventions;

FIG. 16 is a block diagram of a generic optical detection assembly that can be incorporated into the UOT system of FIG. 15;

FIG. 17 is a block diagram of one embodiment of an interferometer used in the UOT system of FIG. 15;

FIG. 18 is a block diagram of one specific embodiment of the optical detection assembly of FIG. 16;

FIG. 19 is a block diagram of the optical detection assembly of FIG. 18, particularly showing the generation and detection of a kth speckle grain of an interference pattern;

FIG. 20 is a block diagram of another specific embodiment of the optical detection assembly of FIG. 16;

FIG. 21 is a block diagram of the optical detection assembly of FIG. 20, particularly showing the generation and detection of a kth speckle grain of an interference pattern;

FIG. 22 is a block diagram of still another specific embodiment of the optical detection assembly of FIG. 16;

FIG. 23 is a block diagram of the optical detection assembly of FIG. 22, particularly showing the generation and detection of a kth speckle grain of an interference pattern;

FIG. 24 is a block diagram of yet another specific embodiment of the optical detection assembly of FIG. 16;

FIG. 25 is a block diagram of the optical detection assembly of FIG. 24, particularly showing the generation and detection of a kth speckle grain of an interference pattern;

FIG. 26 is a perspective view of one embodiment of a camera microchip that can be used in an optical detection assembly;

FIG. 27 is a block diagram of a pixel of the camera microchip of FIG. 26;

FIG. 28 is a block diagram of an optical detection assembly that uses the camera microchip of FIG. 26;

FIGS. 29a and 29b are perspective views of another embodiment of a camera microchip that can be used in an optical detection assembly;

FIG. 30 is a side view of the camera microchip of FIGS. 29a and 29b;

FIG. 31 is a flow diagram of one method used by the UOT system of FIG. 15 to non-invasively measure a physiologically-dependent optical parameter;

FIG. 32a is a timing diagram of one pulsing sequence used by an ultrasound modulating optical tomography (UOT) system constructed in accordance with still another embodiment of the present inventions, to detect a physiologically-dependent optical parameter in a target voxel within an anatomical structure;

FIG. 32b is a timing diagram of another pulsing sequence used by the ultrasound modulating optical tomography (UOT) system to detect a physiologically-dependent optical parameter in a target voxel within an anatomical structure;

FIG. 33a is a pictorial diagram illustrating the blurring effect caused by movement of the ultrasound during the measurement process by the UOT system of FIG. 15;

FIG. 33b is a pictorial diagram illustrating the blurring effect caused by movement of the ultrasound during the measurement process by the UOT system that utilizes the timing diagram of FIG. 32a;

FIG. 34 is a block diagram of an ultrasound modulating optical tomography (UOT) system that utilizes the timing diagram of FIG. 32a in accordance with another embodiment of the present inventions;

FIG. 35a is a block diagram of one embodiment of an interferometer used in the UOT system of FIG. 34;

FIG. 35b is a block diagram of another embodiment of an interferometer used in the UOT system of FIG. 34;

FIG. 35c is a block diagram of still another embodiment of an interferometer used in the UOT system of FIG. 34;

FIG. 35d is a block diagram of yet another embodiment of an interferometer used in the UOT system of FIG. 34;

FIG. 36a is a timing diagram of background light and signal light components generated by the UOT system of FIG. 15;

FIG. 36b is a timing diagram of background light and signal light components generated by the UOT system of FIG. 34;

FIG. 37 is a histogram comparing the population of the signal energy components detected by the UOT systems of FIG. 15 and FIG. 34;

FIG. 38a is a timing diagram of double arbitrarily-shaped light pulses that can be used in the UOT system of FIG. 34, wherein background light is eliminated in the interference light pattern;

FIG. 38b is a timing diagram of double Gaussian-shaped light pulses that can be used in the UOT system of FIG. 34, wherein background light is eliminated in the interference light pattern;

FIG. 39 is a flow diagram of one method used by the UOT system of FIG. 34 to non-invasively measure a physiologically-dependent optical parameter;

FIG. 40 is a block diagram of an ultrasound modulating optical tomography (UOT) system that utilizes the timing diagram of FIG. 32a in accordance with still another embodiment of the present inventions;

FIG. 41a is a block diagram of one embodiment of an interferometer used in the UOT system of FIG. 40;

FIG. 41b is a block diagram of another embodiment of an interferometer used in the UOT system of FIG. 40;

FIG. 41c is a block diagram of still another embodiment of an interferometer used in the UOT system of FIG. 40;

FIG. 41d is a block diagram of yet another embodiment of an interferometer used in the UOT system of FIG. 40; and

FIG. 42 is a flow diagram of one method used by the UOT system of FIG. 40 to non-invasively measure a physiologically-dependent optical parameter.

### DETAILED DESCRIPTION OF THE EMBODIMENTS

The ultrasound modulated optical tomography (UOT) systems described herein utilize the combination of a pulsed ultrasound sequence that tags light propagating through an anatomical structure, and a selective lock-in camera that detects the tagged light (e.g., via parallel speckle detection (PSD)), as opposed to a conventional camera, to provide a highly efficient and scalable scheme that enables detection of highly localized and high spatial resolution UOT signals (e.g., blood-oxygen level dependent signals) at great depth inside a biological specimen, e.g., noninvasively through the entire thickness of the human skull and into the underlying cerebral cortical brain matter. The UOT systems may utilize a specific homodyne interference scheme that enables shot noise limited detection of the signal light. Such UOT signals may be used for, e.g., brain-computer interfacing, medical diagnostics, or medical therapeutics. Although the UOT systems are described herein as being used to measure brain activity for exemplary purposes, such UOT system can be used to measure other anatomical activities of the body.

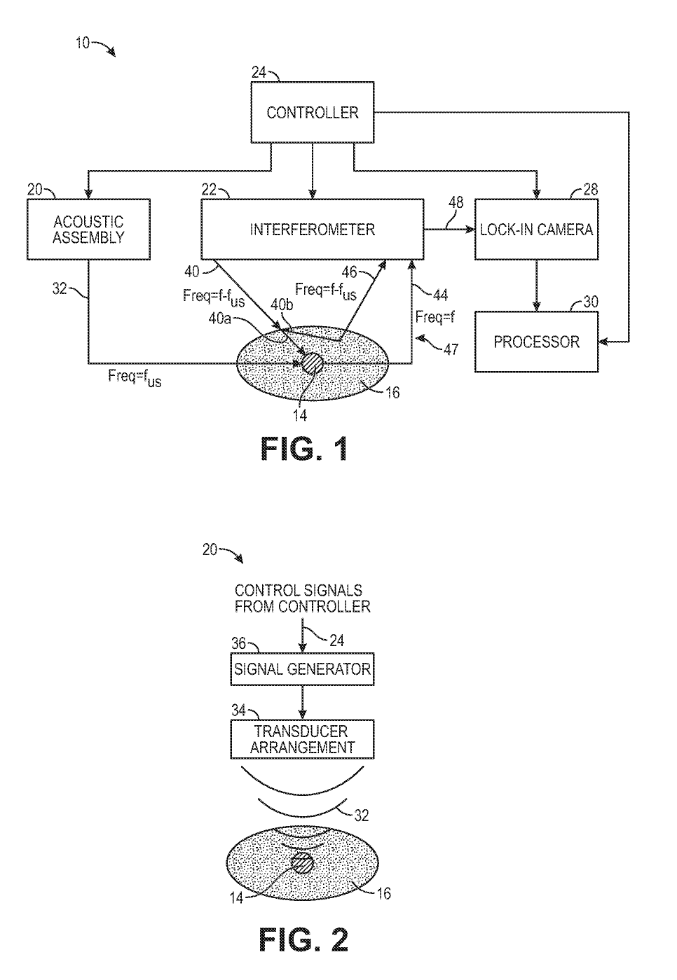

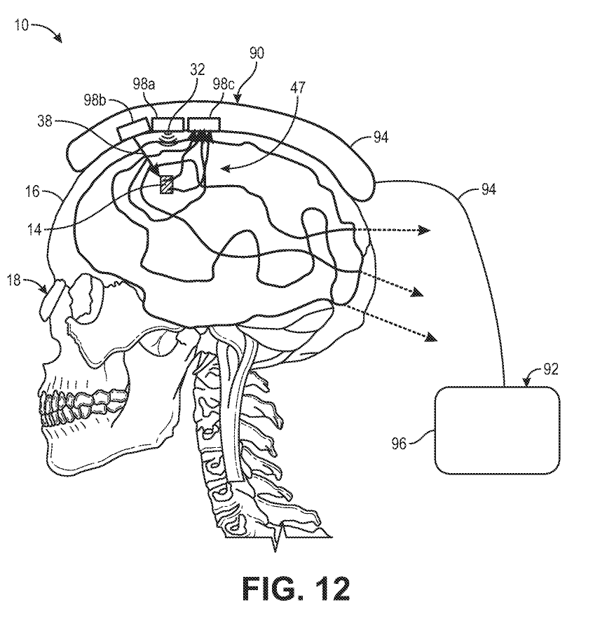

Referring to FIG. 1, an ultrasound modulated optical tomography (UOT) system 10 constructed in accordance with one embodiment of the present inventions will be described. The UOT system 10 is designed to non-invasively measure a physiologically-dependent optical parameter of a target voxel 14 in an anatomical structure 16. In the illustrated embodiment, the anatomical structure 16 is the intact head of a user 18 (shown in FIG. 12), including the scalp, skull, and brain, with the target voxel 14 being a portion of the brain. In a practical implementation, the UOT system 10 will acquire data from multiple target voxels 14 (“data voxels”) spatially separated from each other within a volume of interest (not shown). A “target voxel” may be defined as a small contiguous sub-volume of space (e.g., a cube) within the anatomical structure 16. For purposes of brevity, the UOT system 10 will be described as acquiring one data voxel (i.e., data representative of a physiologically-dependent optical parameter of the target voxel 14), although it should be understood that the UOT system 10 may be capable of acquiring more than one data voxel from the volume of interest of the anatomical structure 16.

In the illustrated embodiment, the physiologically-dependent optical parameter may be, e.g., a level of deoxygenated and/or oxygenated hemoglobin concentration or relative abundance, although in other embodiments, the physiologically-dependent optical parameter can be any parameter that varies in accordance with a change in an optical property of the target voxel 14 (e.g., light absorption). The physiologically-dependent optical parameters may alternatively comprise an analyte concentration in the blood, analyte/metabolite in tissue, concentration of a substance (e.g., blood, hemoglobin) or a structure within tissue, the presence and concentration of lamellar bodies in amniotic fluid for determining the level of lung maturity of a fetus, the presence and/or concentration of meconium in the amniotic fluid, optical properties of other extravascular fluids, such as pleural, pericardial, peritoneal, and synovial fluids. The physiologically-dependent optical parameter may be used internally within the UOT system 10 or may be transmitted to external devices for use therein, e.g., medical devices, entertainment devices, neuromodulation stimulation devices, alarm systems, video games, etc.

The UOT system 10 generally includes an acoustic assembly 20, an interferometer 22, a controller 24, a lock-in camera 28, and a processor 30.

The acoustic assembly 20 is configured for delivering ultrasound 32 into the target voxel 14. Preferably, the acoustic assembly 20 focuses the ultrasound 32 on this target voxel 14 in order to maximize the resolution of the UOT system 10; that is, the more focused the ultrasound 32 is, the smaller the target voxel 14 may be defined, thereby increasing or maximizing the resolution of the UOT system 10.

Preferably, the frequency fᵤₛ of the ultrasound 32 is selected (e.g., in the range of 100 KHz-10 MHz), such that the ultrasound 32 can pass efficiently through the skull and brain matter without significant attenuation that would otherwise cause insufficient ultrasound pressure at the target voxel 14, so that detectable UOT modulation of the light is created, as described in further detail below. It should be appreciated that the wavelength of such ultrasound in brain matter, given that the speed of sound in brain matter is similar to that of water (1500 meter/second), is on the order of fractions of a millimeter to a few millimeters. Thus, the acoustic assembly 20 may obtain ultrasound focal confinement at the target voxel 14 laterally on the order of the wavelength of the ultrasound 32 (e.g., less than 1 mm), and axially on the order of the wavelength of the ultrasound 32 when the acoustic assembly 20 is operated to pulse the ultrasound 32 at short durations (e.g., a single cycle), as will be described in further detail below.

Referring further to FIG. 2, one embodiment of the acoustic assembly 20 includes an ultrasound transducer arrangement 34 and a signal generator 36. The ultrasound transducer arrangement 34 may take the form of any device that emits ultrasound 32 (in the illustrated embodiment, focused ultrasound) at a defined frequency and duration in response to a controlled drive signal; for example, single acoustic element configured for emitting ultrasound beams with a fixed focus; or a piezoelectric phased array capable of emitting ultrasound beams with variable direction, focus, duration, and phase, or may be an array of pressure generating units (e.g., silicon, piezoelectric, polymer or other units), an ultrasound imaging probe, or even an array of laser generated ultrasound (LGU) elements.

The signal generator 36 is configured for generating alternating current (AC) signals for driving the ultrasound transducer arrangement 34 at a defined ultrasound frequency, duration, and intensity. The AC drive signal may be electrical or optical, depending on the nature of the ultrasound transducer arrangement. The signal generator 36 includes control inputs (not shown) for receiving control signals from the controller 24 that cause the ultrasound transducer arrangement 34 to emit the ultrasound 32 at a selected time, duration, and intensity. Thus, as will be described in further detail below, the controller 24 may selectively pulse the ultrasound 32.

In one particular embodiment, the transducer arrangement 34 is a head-mounted steerable ultrasonic array coupled to the skin of the user via hydrogel or other means of mechanical coupling in order to effectively launch the ultrasound 32 towards the precisely defined target voxel 14 within the anatomical structure 16, and in this case, the three-dimensional volume of the brain, while compensating the ultrasound wavefront using well-known phased array techniques to achieve efficient and selective ultrasound delivery to the target voxel 14.

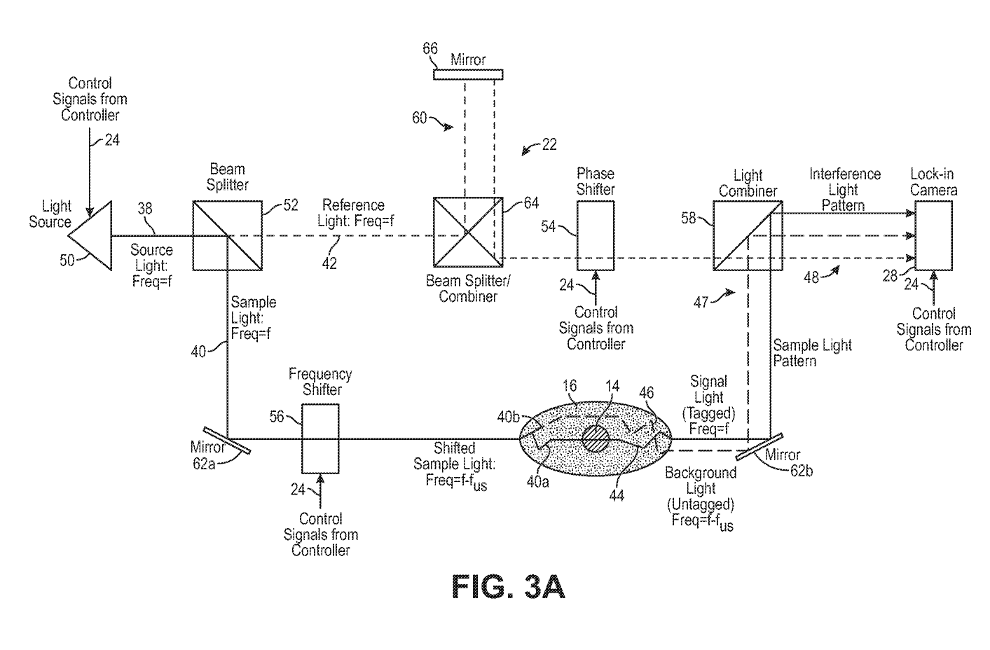

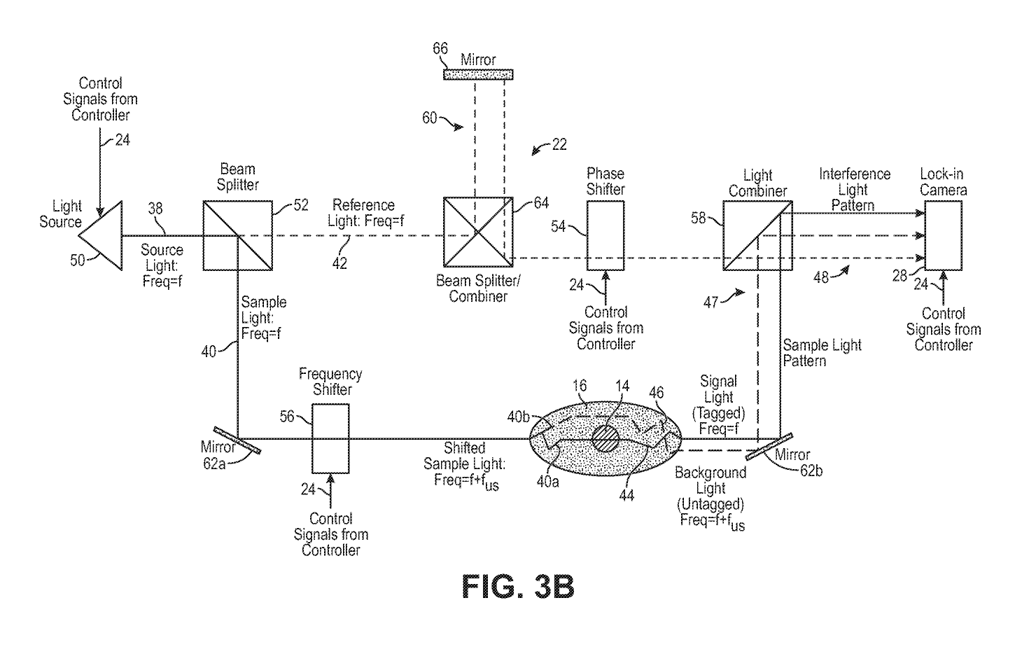

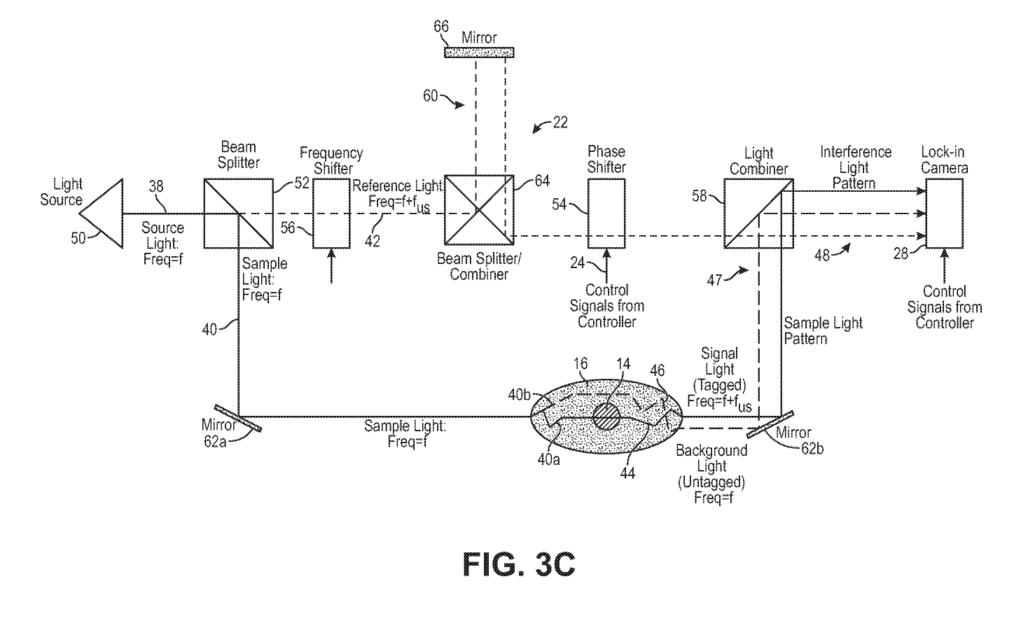

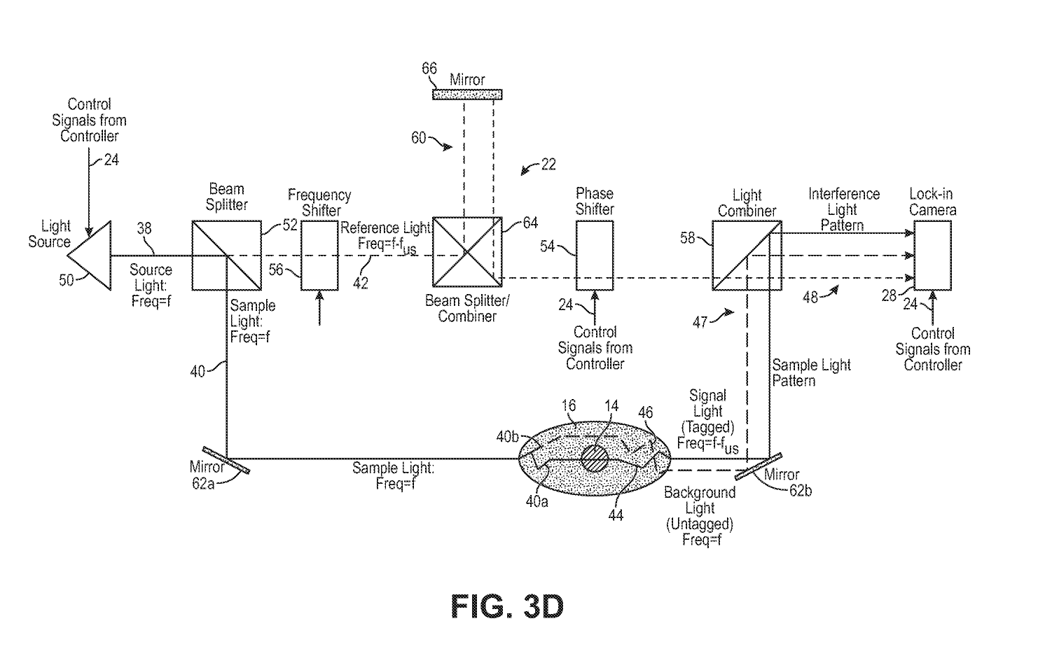

Referring to FIGS. 1 and 3, the interferometer 22 is configured for delivering sample light 40 into the anatomical structure 16, where the sample light scatters diffusively, e.g., through the human skull, into the brain, and back out again. Thus, a portion 40a of the sample light 40 will pass through the target voxel 14 and will be scattered by the anatomical structure 16 as signal light 44, and another portion 40b of the sample light 40, which does not pass through the target voxel 14, and will be scattered by the anatomical structure 16 as background light 46. The signal light 44 and background light 46 combine together to create a sample light pattern 47 that exits the anatomical structure 16. The interferometer 22 is further configured for combining reference light 42 with the sample light pattern 47 to generate an interference light pattern 48 (e.g., speckle light pattern, which can be defined as an intensity pattern produced by the mutual interference of a set of scattered wavefronts; that is, a speckle light pattern results from the interference of many waves, but having different phases and amplitudes, which add together to give a resultant wave whose amplitude, and therefore intensity and phase, varies randomly). In the illustrated embodiment, the interferometer 22 is configured for splitting source light 38 into the sample light 40 and reference light 42, as will be described in further detail below.

The reference light 42 may be combined with the signal light 44 in the sample light pattern 47 in a homodyne manner, e.g., by initially frequency shifting the sample light 40 by the frequency fᵤₛ of the ultrasound 32 delivered into the target voxel 14 by the acoustic assembly 20. For purposes of this disclosure, the term “homodyne,” when referring to the combination of signal light and reference light, means that the signal light and reference light have the same frequency when combined to generate interference terms of the same frequency, as opposed to the term “heterodyne,” which means that the signal light and reference light have different frequencies when combined to generate interference terms with beat frequencies.

It should be appreciated that, if unmodified, the sample light portion 40a passing through the target voxel 14 will be frequency shifted (i.e., tagged) by the ultrasound 32 that also passes through the target voxel 14, such that the signal light 44 will have a frequency f−fᵤₛ. Presumably, the sample light portion 40b not passing through the target voxel 14 will not be frequency shifted (i.e., untagged) by the ultrasound 32, such that the background light 46 will have a frequency f, i.e., the frequency of the sample light 40. It is also noted that not all of the sample light portion 40a passing through the target voxel 14 will be tagged by the ultrasound 32 (i.e., there exists a tagging efficiency (i.e., the number of tagged photons relative to a number of untagged photons scattered by the target voxel 14)), and therefore, some of the sample light portion 40a passing through the target voxel 14 will be scattered by the anatomical structure 16 as background light 46.

However, assuming that the reference light 42 and the sample light 40 output by the interferometer 22 have the same frequency f, in order to combine the ultrasound tagged signal light 44 in the sample light pattern 47 and the reference light 42 in a homodyne manner, which requires the reference light 42 and signal light 44 to have the same frequency, the frequency f of the sample light 40 or the reference light 42 must initially be shifted relative to each other by the ultrasound frequency fᵤₛ, such that, upon combining by the interferometer 22, the frequency of the ultrasound tagged signal light 44 will be shifted to the same frequency as the reference light 42, and the frequency of the untagged background light 46 will differ from the frequency of the reference light 42 by the ultrasound frequency fᵤₛ. Thus, either the sample light 40 or the reference light 42 will be pre-conditioned, such that the ultrasound tagged signal light 44 will interfere with the reference light 42 in a homodyne manner, resulting in a DC interference component between the reference light 42 and signal light 44 that can be detected by the lock-in camera 28 as the signal component during each pulse, as will be described in further detail below. In contrast, the frequency shifting of the sample light 40 before it enters the anatomical structure 16, or the frequency shifting of the reference light 42, will prevent the untagged background light 46 from interfering with the reference light 42 in a homodyne manner.

In the embodiment illustrated in FIG. 1, the interferometer 22 down frequency shifts the sample light 40 by the ultrasound frequency fᵤₛ to f−fᵤₛ, such that the ultrasound tagged signal light 44 has the frequency fᵤₛ, the untagged background light 46 has the frequency f−fᵤₛ, and the reference light 42 has a frequency fᵤₛ, thereby enabling combination of the reference light 42 and signal light 44 in a homodyne manner, as further described below with respect to FIG. 3a. However, it is noted that because the ultrasound 32 will tag the signal light 44 with the ultrasound frequencies +fᵤₛ and −fᵤₛ, as well as other positive and negative multiples of the ultrasound frequency fᵤₛ, other frequency shifting implementations are possible to effect homodyne combination of the reference light 42 and signal light 44. For example, as described in further detail below, the interferometer 22 may up frequency shift the sample light 40 by the ultrasound frequency fᵤₛ to f+fᵤₛ, such that the ultrasound tagged signal light 44 has the frequency fᵤₛ, the untagged background light 46 has the frequency f+fᵤₛ, and the reference light 42 has a frequency fᵤₛ (see FIG. 3b); may up frequency shift the reference light 42 by the ultrasound frequency fᵤₛ to f+fᵤₛ, such that the ultrasound tagged signal light 44 has the frequency f+fᵤₛ, the untagged background light 46 has the frequency f, and the reference light 42 has a frequency f+fᵤₛ, (see FIG. 3c); may down frequency shift the reference light 42 by the ultrasound frequency fᵤₛ to f−fᵤₛ, such that the ultrasound tagged signal light 44 has the frequency f−fᵤₛ, the untagged background light 46 has the frequency f, and the reference light 42 has a frequency f−fᵤₛ (see FIG. 3d); or perform any other frequency shift of the sample light 40 or reference light 42 that results in the homodyne combination of the reference light 42 and the signal light 44.

The interferometer 22 is further configured for modulating (and in the illustrated embodiment, phase modulating) the interference light pattern to generate a plurality of different interference light patterns, which as will be described in further detail below, enables the amplitude of the signal light 44 to be distinguished from the background light 46.

Referring further to FIG. 3a, one embodiment of the interferometer 22 includes a light source 50, a beam splitter 52, an optical phase shifter 54, an optical frequency shifter 56, a light combiner 58, a path length adjustment mechanism 60, and a set of mirrors 62a, 62b (generally, 62).

The light source 50 is configured for generating coherent light as the source light 38, preferably at a single wavelength (e.g., in the range of 605 nm to 1300 nm), and may take the form of, e.g., a laser diode. In alternative embodiments, multiple light source(s) (not shown) may be used to generate the source light 38 at multiple distinct wavelengths, e.g., one generating source light 38 within the range of 605 nm to 800 nm, and another generating source light 38 within the range of 800 nm to 1300 nm. The coherence length of the source light 38 is preferably at least one meter in order to generate the best speckle contrast in the speckle light pattern 48. The light source 50 may receive power from a drive circuit (not shown), which may include control inputs for receiving control signals from the controller 24 that cause the light source 50 to emit the source light 38 at a selected time, duration, and intensity. Thus, as will be described in further detail below, the controller 24 may selectively pulse the source light 38, and thus the sample light 40 and reference light 42.

As specifically illustrated in FIG. 3a, the beam splitter 52 is configured for splitting the source light 38 into the sample light 40 that propagates along a sample arm of the interferometer 22 and reference light 42 that propagates along a reference arm of the interferometer 22. In the illustrated embodiment, the beam splitter 52 (e.g., a partially transparent mirror) splits the source light 38 via amplitude division by reflecting a portion of the source light 38 as the sample light 40, and transmitting the remaining portion of the source light 38 as the reference light 42, although the beam splitter 52 may alternatively reflect a portion of the source light 38 as the reference light 42, and transmit the remaining portion of the source light 38 as the sample light 40. In alternative embodiments, the beam splitter 52 may split the source light 38 via wavefront division by splitting a portion of the wavefront into the sample light 40 and splitting the remaining portion of the wavefront into the reference light 42. In either case, the beam splitter 52 may not necessarily split the source light 38 equally into the sample light 40 and reference light 42, and it may actually be more beneficial for the beam splitter 52 to split the source light 38 unevenly, such that the amplitude of the sample light 40 is less than the amplitude of the reference light 42 (e.g., 10/90 power ratio) in order to comply with tissue safety standards. That is, the amplitude of the sample light 40 will preferably be relatively low to avoid damaging the tissue, whereas the amplitude of the reference light 42, which will be used to boost the signal light 44 in the interference light pattern 48, will be relatively high.

The optical phase shifter 54 is configured for setting the phase difference between the sample light 40 and reference light 42. The optical phase shifter 54 may include control inputs (not shown) for receiving control signals from the controller 24 that cause the optical phase shifter 54 to set the phase of the reference light 42 relative to the sample light 40. Thus, as will be described in further detail below, the controller 24 may selectively set the phase between the sample light 40 and the reference light 42.

The optical frequency shifter 56 is configured for down frequency shifting the sample light 40 by the ultrasound frequency fᵤₛ to f−fᵤₛ such that the frequency of the ultrasound tagged signal light 44 will be f, while the frequency of the background light 46 will be f−fᵤₛ, thereby enabling the homodyne combination of the reference light 42 at frequency f and the ultrasound tagged signal light 44 at frequency f, as described above with respect to FIG. 1. In one alternative embodiment illustrated in FIG. 3b, the optical frequency shifter 56 is configured for up frequency shifting the sample light 40 by the ultrasound frequency fᵤₛ to f+fᵤₛ, such that the frequency of the ultrasound tagged signal light 44 will be f, while the frequency of the background light 46 will be f+fᵤₛ, thereby enabling the homodyne combination of the reference light 42 at frequency f and the ultrasound tagged signal light 44 at frequency f. In one alternative embodiment illustrated in FIG. 3c, the optical frequency shifter 56 is configured for up frequency shifting the reference light 42 by the ultrasound frequency fᵤₛ to f+fᵤₛ, such that the frequency of the ultrasound tagged signal light 44 will be f+fᵤₛ, while the frequency of the background light 46 will be f, thereby enabling the homodyne combination of the reference light 42 at frequency f+fᵤₛ and the ultrasound tagged signal light 44 at frequency f+fᵤₛ. In still another alternative embodiment illustrated in FIG. 3d, the optical frequency shifter 56 is configured for down frequency shifting the reference light 42 by the ultrasound frequency fᵤₛ to f−fᵤₛ, such that the frequency of the ultrasound tagged signal light 44 will be f−fᵤₛ, while the frequency of the background light 46 will be f, thereby enabling the homodyne combination of the reference light 42 at frequency f−fᵤₛ and the ultrasound tagged signal light 44 at frequency f−fᵤₛ.

In any event, the frequency shifter 54 may include a local oscillator (not shown) that outputs a signal having a fixed or variable frequency. The local oscillator may be variable, in which case, it may have a control input for receiving control signals from the controller 24 that cause the local oscillator to output a signal at a defined frequency. Alternatively, the local oscillator may be fixed, in which case, it will output a signal having a fixed frequency. In either case, the frequency of the signal output by the local oscillator will be equal to the frequency fᵤₛ of the ultrasound 32 emitted by the acoustic assembly 20.

The light combiner 58 is configured for combining the reference light 42 with the sample light pattern 47 via superposition to generate the interference light pattern 48. The light combiner 58 can take the form of, e.g., a combiner/splitter mirror.

The path length adjustment mechanism 60 is configured for adjusting the optical path length of the reference light 42 (i.e., the reference arm) to nominally match the expected optical path length of the combined sample light 40 and signal light 44 (i.e., the sample arm), such that the signal light 44 and the reference light 42 reach the light combiner 58 at the same time. The path length adjustment mechanism 60 may include a beam splitter/combiner 64 and an adjustable mirror 66 that can be displaced relative to the beam splitter/combiner 64. The beam/splitter combiner 64 is configured for redirecting the reference light 42 at a ninety-degree angle towards the mirror 66, and redirecting the reference light 42 reflected back from the mirror 66 at a ninety-degree angle towards the light combiner 58. Thus, adjusting the distance between the mirror 66 and the beam splitter/combiner 64 will adjust the optical path length of the reference arm to match the optical path length of the sample arm.

The mirror assembly 62 is configured for confining the optical light paths in the interferometer 22 into a small form factor, and in the illustrated embodiment, includes a first tilted, completely reflective, mirror 62a configured for redirecting the sample light 40 at a ninety-degree angle towards the biological specimen 16, and a second tilted, completely reflective, mirror 62b configured for redirecting the signal light 44 (and coincidentally a portion of the background light 46) towards the light combiner 58.

Referring back to FIG. 1, the controller 24, which may, e.g., take the form of a central processing unit (CPU), is configured for implementing pulsed wave (PW) UOT by operating the acoustic assembly 20 to pulse the ultrasound 32 (in the illustrated embodiment, by sending on/off control signals to the signal generator 36), and operating the interferometer 22 to pulse the sample light 40 (in the illustrated embodiment, by sending on/off control signals to the drive circuit coupled to the light source 50) in synchrony at the target voxel 14, with the (comparatively very slow) flight of the ultrasound 32, such that only the signal light 44 is frequency shifted (i.e., tagged) by the ultrasound 32. That is, a pulse of the sample light 40 will be delivered into the anatomical structure 16, such that it will pass through the target voxel 14 only as the ultrasound 32 passes through the target voxel 14. In this manner, no portion of the background light 46 will be tagged by the ultrasound 32. As a result, pulsed wave (PW) UOT improves the spatial resolution in the axial direction (or depth) compared to continuous wave (CW) UOT. Thus, PW UOT achieves axial confinement and three-dimensional (3D) spatial resolution, rather than merely two-dimensional (2D) spatial resolution as in the case with CW UOT.

The controller 24 is further configured for operating the interferometer 22 to sequentially modulate the interference light pattern 48 (in the illustrated embodiment, by sending on/off control signals to the optical phase shifter 54) to generate a plurality of different interference light patterns. As will be described in further detail below, the interferometer 22 will set different phases (and in the illustrated embodiment, four different phases equal to 0, π/2, π, and 3π/2) between sequential pulses of the sample light 40 and the reference light 42 to facilitate quadrature detection of the signal light 44. As will be also described in further detail below, the controller 24 is further configured for synchronously operating the lock-in camera 28, such that the bin shifting of data detected by the lock-in camera 28 is performed in synchrony with the phase changes in the interferometer 22.

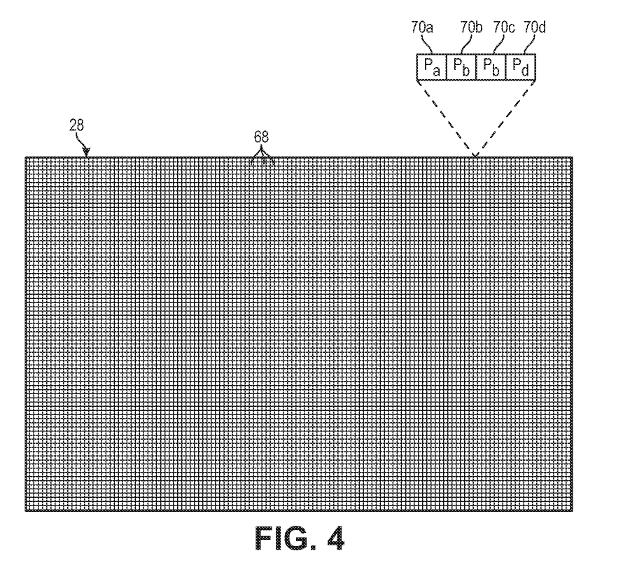

Referring further to FIG. 4, the lock-in camera 28 includes an array of detectors 68 (or “pixels”) configured for simultaneously detecting spatial components of each of the different interference light patterns 48. In the case where the interference light pattern 48 is a speckle light pattern, the spatial components are speckle grains (approximately the size of a wavelength of the light) of the speckle light pattern. In general, lock-in cameras include a class of digital cameras in which multiple measurements of a light field are rapidly made at each pixel in a temporally precise fashion synchronized with an external trigger or oscillation and stored in multiple “bins” within each pixel, in contrast with conventional cameras, which store only one value per pixel that merely aggregate the incoming photo-electrons over the camera frame integration time. Lock-in cameras may also perform on-chip computations on the binned values. Thus, the key feature of lock-in cameras is their ability to rapidly capture and store multiple sequential samples of the light field, with sample-to-sample latencies shorter than readout times of conventional cameras. This feature enables them, for example, to sample a modulated light field at the same frequency as the modulation, such that subtraction across successive samples, or other operations, such as quadrature detection (discussed below) will extract the component of the light that is modulated at the modulation frequency, while subtracting off the unmodulated (“DC”) background. Similarly, lock-in cameras can be used to make a series of such measurements or comparisons, locked to an external trigger signal (generated by the controller 24), rapidly in order to extract such modulated components from a rapidly changing light field arising from a dynamic, disordered biological specimen.

Thus, each detector 68 of the lock-in camera 28 respectively stores a plurality of values in a plurality of bins 70a-70d representative of the spatial component of the four interference light patterns 48, and in this case, four bins 70a-d (in general, 70) for storing four values from the respective four interference light patterns 48. The spatial component values stored in the bins 70 of a respective detector 68 may be, e.g., the intensity values of the respective spatial component of interference light patterns 48. For example, for any particular detector 68 (or pixel) corresponding to a particular spatial component (or speckle grain), four power values Pₐ-P_{d} for the four interference patterns 48 will be respectively stored in the four bins 70a-70d. As will be described in further detail below, the spatial component power values Pₐ-P_{d} detected by each detector 68 of the camera 28 for the four interference patterns 48 can be used to reconstruct the amplitude of the signal light 44, and thus, can be said to be representative of the physiologically-dependent optical parameters (e.g., optical absorption) of the target voxel 14. The lock-in camera 28 includes control inputs (not shown) for receiving control signals from the controller 24, such that the detection and binning of the data can be coordinated with the pulsing of the ultrasound 32 and sample light 40 described in further detail below.

Although only a single lock-in camera 28 is illustrated, it should be appreciated that multiple lock-in cameras 28 (e.g., in an array) or a lock-in camera in the form of multiple camera sensor chips on a common circuit board, can be used to increase the number of detectors 68 (i.e., pixels). Although not illustrated, the system 10 may include magnification optics and/or apertures to magnify the individual speckle grains, which may have a size on the order of the wavelength of the near-infrared or visible light used to acquire the data voxel, and hence on the order of hundreds of nanometers in size, to approximately the sizes of the detectors 68 of the lock-in camera 28. Thus, in the illustrated embodiment, the pixel sizes and pitches of the lock-in camera 28 are matched to the speckle grain sizes and pitches of the interference light pattern 48 via the appropriate magnification, although other embodiments are possible.

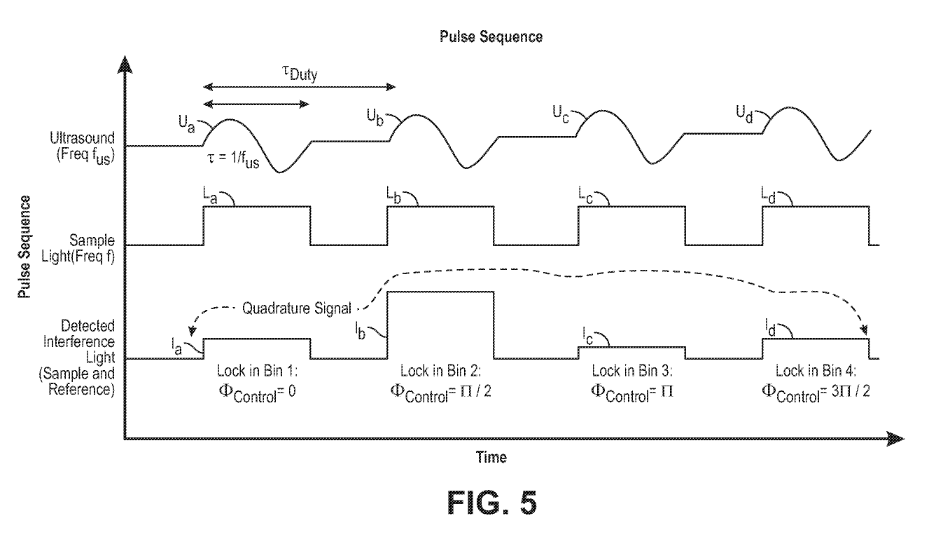

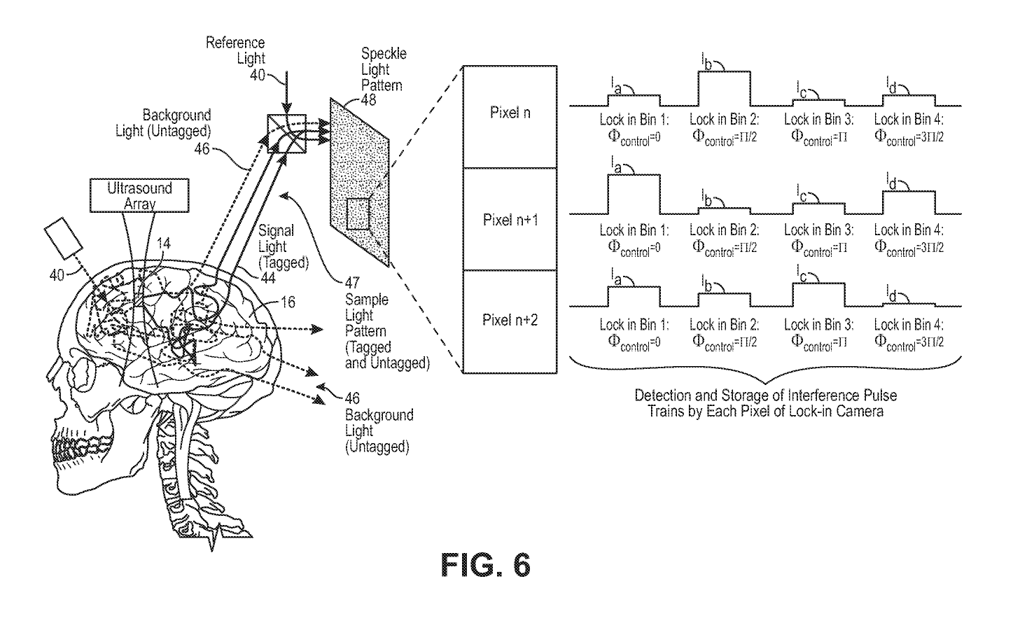

Referring to FIGS. 5 and 6, one pulsing sequence that can be used in a PW UOT technique performed by the system 10 for generating four interference light patterns 48 and detecting and storing the spatial component power values for the interference light patterns 48 will be described.

During one acquisition of a single data voxel (i.e., acquisition of data characterizing the target voxel 14), an ultrasound pulse train consisting of four separate, but identical, ultrasound pulses Uₐ-U_{d} are delivered into the target voxel 14. In this embodiment, the duration T of each ultrasound pulse U is equal to only one full cycle of the ultrasound 32 to maximize the data acquisition speed, and thus, is equal to 1/fᵤₛ although in alternative embodiments, the duration T may be several ultrasound cycles long (e.g., on the order of 1 microsecond or less than one microsecond). It should be noted that it is desirable to minimize the duration T of the ultrasound pulse U in order to minimize ultrasound focal confinement at the target voxel 14.

The duty cycle of the ultrasound pulses Uₐ-U_{d} (i.e., the time that elapses between the beginning of one pulse U to the beginning of the next pulse U) is τ_{duty}. The duty cycle τ_{duty} may be selected to allow each ultrasound pulse U to exit the anatomical structure 16 before the next measurement is taken, such that the ultrasound tagged signal light 44 is only present at high pressures at the three-dimensional location of the target voxel 14. The frame rate of the lock-in camera 28 should be selected to match the duty cycle τ_{duty} of the ultrasound pulse U, such that there exists one ultrasound pulse U per frame.

A light pulse train consisting of four sample light pulses Lₐ-L_{d} is also delivered into the anatomical structure 16 in synchrony with the delivery of the four ultrasound pulses UₐU_{d}, at the target voxel 14, such that, as each ultrasound pulse U passes through the target voxel 14, the sample light pulse L likewise passes through the target voxel 14. It should be appreciated that although the sample light pulses are illustrated herein as being rectangular-shaped for purposes of brevity, other shapes for the sample light pulses can be used, including, e.g., triangular or Gaussian.

In this manner, only the signal light 44 (and none of the background light 46) is tagged with the ultrasound, as discussed above. In this particular embodiment, only one sample light pulse L is delivered for each ultrasound pulse U. Thus, there is a one-to-one correspondence between the sample light pulses LₐL_{d} and the ultrasound pulses UₐU_{d}. Although each of the sample light pulses L is illustrated in FIG. 5 as having the same width as the width of the respective ultrasound pulses U for purposes of illustration, each sample light pulses L is, in practicality, at least slightly smaller than the respective ultrasound pulse U due to the latency period required for the respective ultrasound pulse to reach the target voxel 14. Alternatively, the duration of the sample light pulse L can be much less than the duration of the ultrasound pulse U. In any event, the duration of the sample light pulse L preferably should be approximately matched to be an integer multiple of the frequency of the ultrasound frequency fᵤₛ, e.g., if the ultrasound frequency fᵤₛ of 2 MHz, the duration of the sample light pulse L should be multiples of 0.5 microseconds. Although FIG. 5 illustrates only one ultrasound cycle per ultrasound pulse U, if the duration of the sample light pulse L is multiple integers of the frequency of the ultrasound frequency fᵤₛ, the number ultrasound cycles per ultrasound pulse U is preferably equal to the same multiple integer. This ensures that a full, balanced cycle of tagged light is generated. The energy of each sample light pulse L should be sufficiently high, e.g., on the order of 1 microsecond in duration (but can be as low as 10 nanoseconds in duration) and tens of micro-Joules per square centimeter.

For each of the four separate ultrasound pulses Uₐ-U_{d} occurring during the acquisition of a single data voxel, the phase difference between the reference light 42 and the sample light 40 is set to a different setting, and in this case, to one of 0, π/2, π, and 3π/2. In the illustrated embodiment, the phase between the reference light 42 and the sample light 40 is sequentially set to 0, π/2, π, and 3π/2, although these phase settings can be performed in any order, as long as all four phase settings 0, π/2, π, and 3π/2 are used during the acquisition of a single data voxel.

Although a single optical pulse L is shown for each ultrasound pulse U, a series of pulses L, all set with the same phase difference between the reference light 42 and the sample light, can be generated during a respective ultrasound pulse U (e.g., two light pulses Lₐ for the ultrasound pulse Uₐ when the phase between the reference light 42 and the sample light 40 is set to 0, two light pulses L_{b} for the ultrasound pulse U_{b} when the phase between the reference light 42 and the sample light 40 is set to π/2, two light pulses L_{c} for the ultrasound pulse U_{c} when the phase between the reference light 42 and the sample light 40 is set to π, and two light pulses L_{d} for the ultrasound pulse U_{d} when the phase between the reference light 42 and the sample light 40 is set to 3π/2.

Irrespective of the number of same-phased optical pulses L generated during each ultrasound pulse U, the respective pulses of the sample light pattern 47 and reference light 42 are then combined into the interference light patterns 48, each having four corresponding interference pulses Iₐ-I_{d} that can be detected by the lock-in camera 28. That is, for each interference pulse I, a detector 68 detects a spatial component of the respective interference pulse I (e.g., a speckle grain in the case where the interference light pattern 48 includes a speckle pattern) and stores the spatial component value (e.g., power) within a respective one of the bins 70.

That is, at phase φ=0, a given pixel n will detect and store the value of the respective spatial component of the interference pulse Iₐ into bin 1 of the pixel n; at phase φ=π/2, the pixel n will detect and store the value of the respective spatial component of the interference pulse I_{b} into bin 2 of the pixel n; at phase φ=η, the pixel n will detect and store the value of the respective spatial component of the interference pulse I_{c} into bin 3 of the pixel n; and at phase φ=3π/2, the pixel n will detect and store the value of the respective spatial component of the interference pulse I_{d} into bin 4 of the pixel n.

Similarly, at phase φ=0, the next pixel n+1 will detect and store the value of the respective spatial component of the interference pulse Iₐ into bin 1 of the pixel n+1; at phase φ=π/2, the pixel n+1 will detect and store the value of the respective spatial component of the interference pulse I_{b} into bin 2 of the pixel n+1; at phase φ=π, the pixel n+1 will detect and store the value of the respective spatial component of the interference pulse I_{c} into bin 3 of the pixel n+1; and at phase φ=3π/2, the pixel n+1 will detect and store the value of the respective spatial component of the interference pulse I_{d} into bin 4 of the pixel n+1.

Similarly, at phase φ=0, the next pixel n+2 will detect and store the value of the respective spatial component of the interference pulse Iₐ into bin 1 of the pixel n+2; at phase φ=π/2, the pixel n+2 will detect and store the value of the respective spatial component of the interference pulse I_{b} into bin 2 of the pixel n+2; at phase φ=π, the pixel n+2 will detect and store the value of the respective spatial component of the interference pulse I_{c} into bin 3 of the pixel n+2; and at phase φ=3π/2, the pixel n+2 will detect and store the value of the respective spatial component of the interference pulse I_{d} into bin 4 of the pixel n+2.

Thus, for each of an n number of pixels, four values will be respectively stored in the four bins 1-4. Significantly, in the case where the interference light pattern 48 includes a speckle light pattern, it is important that all four sample light pulses P be delivered by the interferometer 22 to the target voxel 14 and that all four interference pulses I be detected and recorded by the camera 28 within the characteristic speckle decorrelation time of the target voxel 14, which scales super-linearly with the depth into the anatomical structure 16 at which the target voxel 14 is located. For taking measurements from deep inside a living biological tissue, such as through the human skull and into the human cerebral cortex, the speckle decorrelation time is expected to be on the order of microseconds to tens of microseconds. For taking measurements directly into living brain matter in the absence of skull, speckle decorrelation times have been measured to be on the order of ten milliseconds for 1-millimeter penetration or 1-millisecond for 3-millimeter penetration. Notably, the speckle decorrelation time impacts the depth scaling of lock-in camera based UOT in dynamic scattering media, such as biological tissue, namely the constraint that multiple phase-shifted measurements must be made within the speckle decorrelation time (see, e.g., Qureshi M M, Brake J., Jeon H J, Ruan H, Liu Y, Safi A M, Eom T J, Yang C., Chung E, “In Vivo Study of Optical Speckle Decorrelation Time Across Depths in the Mouse Brain,” Biomedical Optics Express, Vol. 8, No. 11, pp. 4855-4864 (Nov. 1, 2017). Thus, it is important that the time window in which the set of quadrature measurements is short enough that the target voxel 14 does not have the time to de-correlate significantly. Otherwise, the signal-to-noise ratio is diminished.

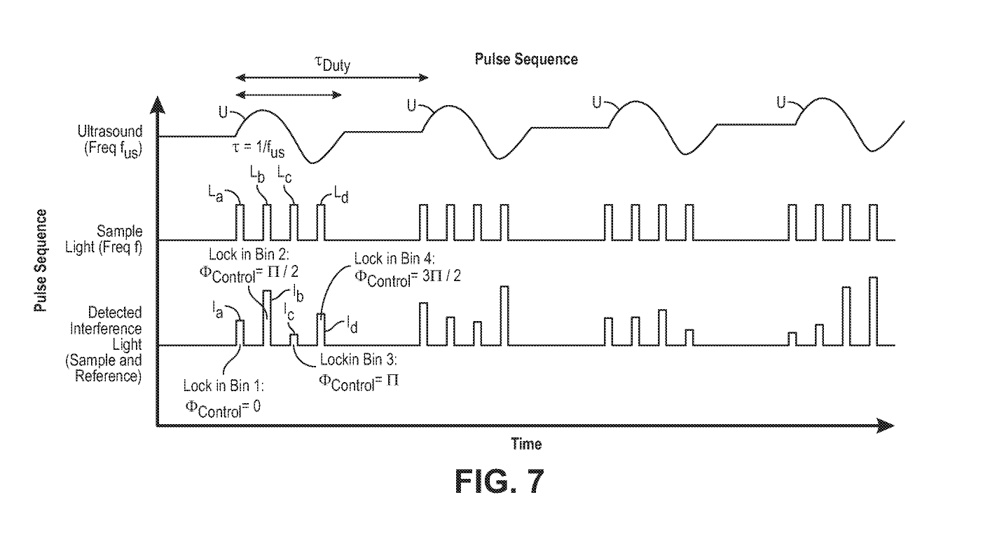

Referring to FIG. 7, another particularly advantageous pulsing sequence that can be used in a PW UOT technique performed by the system 10 for generating four interference light patterns 48 and detecting and storing the spatial component power values for the interference light patterns 48 will be described. The pulsing sequence of FIG. 7 is identical to the pulsing sequence of FIG. 5, with the exception that multiple sample light pulses L (for all phase settings), and in this embodiment, all four sample light pulses L (for the four phase settings), are delivered to the target voxel 14 for each ultrasound pulse U delivered to the target voxel 14, thereby accelerating the delivery of the sample light pulses P by the interferometer 22 to the target voxel 14, and the resultant generation, detection, and recording of all four interference pulses I by the camera 28. That is, because the four sample light pulses L are delivered for each ultrasound pulse U, the speed of the data voxel acquisition is increased by a factor of four.

In particular, during the acquisition of four consecutive data voxels (as opposed to only one in the pulsing sequence of FIG. 5), an ultrasound pulse train consisting of four separate, but identical, ultrasound pulses U are delivered into the target voxel 14. As with the case in the pulsing sequence of FIG. 5, the duration T of this ultrasound pulse U is equal to only one full cycle of the ultrasound 32 to maximize the data acquisition speed, and thus, is equal to 1/fᵤₛ. The duration T of this ultrasound pulse U and the duty cycle τ_{duty} of the ultrasound train pulse of FIG. 7 can be identical to the respective duration T and duty cycle τ_{duty} of the ultrasound pulse train in the pulsing sequence of FIG. 7.

A light pulse train consisting of four sets of sample light pulses, with each set comprising four sample light pulses Lₐ-L_{d}, are also delivered into the anatomical structure 16 in synchrony with the delivery of the four ultrasound pulses U, at the target voxel 14, such that as each ultrasound pulse U passes through the target voxel 14, the corresponding set of four sample light pulses LₐL_{d}, likewise pass through the target voxel 14. Thus, only the signal light 44 (and none of the background light 46) is tagged with the ultrasound, as discussed above. Thus, four sample light pulses LₐL_{d} are delivered for each ultrasound pulse U. Thus, there is a four-to-one correspondence between the sample light pulses LₐL_{d} and ultrasound pulses U.

Thus, in the same manner described above with respect to the pulsing sequence illustrated in FIG. 5, for each ultrasound pulse U occurring during the acquisition of a single data voxel, the phase difference between the reference light 42 and the sample light 40 is set to a different setting, and in this case, to one of 0, π/2, π, and 3π/2, to generate four interference pulses Iₐ-I_{d}. The quick detection and storage scheme of the lock-in camera 28 enables acquisition of an entire data voxel within one cycle of the ultrasound 32, well within the speckle decorrelation time of the target voxel 14.

It can be appreciated that the use of the lock-in camera 28 provides for a high-speed and precisely timed detection method that can capture differences in a light field far faster than the frame rates of conventional cameras. In the illustrated embodiment, the lock-in camera 28 rapidly measures the four quadratures of the pulse sequences illustrated in FIGS. 5 and 7. The acquisition sequence can be precisely timed to external signals for integration with optimal ultrasound and light pulse sequences. The lock-in camera 28 also enables an efficient detection scheme compared to conventional pulsed UOT, since the pulsed UOT signals may be quickly detected with a high signal-to-noise ratio, while using the full bit depth of the analog-digital conversion available in the signal chain due to rejection of DC background by the lock-in camera 28. The lock-in camera 28 provides for a simplified detection scheme that is highly scalable to large numbers of pixels and high frame rates, enabling the maximization of signal capture in UOT, thereby improving spatial and temporal resolution.

It should be appreciated that in addition to the ability of the combination of the pulsed UOT with a lock-in camera to provide high axial spatial resolution and high sensitivity from the high-speed lock-in detection, such combination also provides the additional advantage of efficiently detecting the signal light associated with a specific time point on the ultrasound phase cycle (e.g., at the peaks of the ultrasound phase cycle). As such, the pulsed UOT/lock-in camera combination can accurately measure tissue with a relatively small number of data measurements, and thus, a relatively short period of time, preferably within the speckle decorrelation time of the target voxel. In comparison, a continuous wave approach results in averaging light signal detection over a range of arbitrarily placed points on the ultrasound phase cycle, leading to a diminished overall detection sensitivity, requiring that, for sufficient sensitivity, data measurements be taken over a period time longer than the speckle decorrelation time of the target voxel. Thus, the use of pulsed UOT in combination with the lock-in camera allows for deeper measurements into tissue.

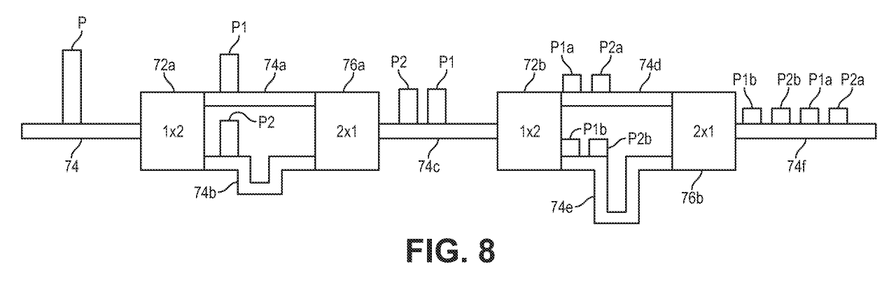

The detection processes illustrated in FIGS. 5 and 7 require the ultrasound timing and intensity to be consistent. As such, the output of the acoustic assembly 20 may be periodically sampled to ensure that the system 10 does not suffer from ultrasound performance drift, for instance, as the transducer arrangement 34 heats. Furthermore, the detection processes illustrated in FIGS. 5 and 7 require that all of the light pulses used between phase changes in the quadrature measurements be equal in strength, or at least exhibit a known ratio that can be normalized out post-detection. In the case that the pulse-to-pulse variations in the light intensity emitted by the light source 50 are too large, the optical arrangement illustrated in FIG. 8 can be utilized to generate optical pulses that are identical in intensity and intensity-time profile, but temporally separated from each other.

In particular, this optical arrangement includes a first 1×2 fiber splitter 72a in which a single optical pulse P (generated by the light source 50) is input via an optical fiber 74 and split into two identical optical pulses P1, P2. Two optical fibers 74a, 74b of different optical lengths are connected to the respective outputs of the first 1×2 fiber splitter 72a, such that the two identical optical pulses P1, P2 respectively propagate within the two optical fibers 74a, 74b. The optical arrangement further includes a first 2×1 fiber coupler 76a into which the two identical optical pulses P1, P2 are input and combined, and output to a single optical fiber 74c. By making the lengths of the optical fibers 74a, 74b different from each other, the single optical pulse P input into the first 1×2 fiber splitter 72a is effectively split into two identical optical pulses that propagate through the single optical fiber 74c and are spaced out by a time difference determined by the optical path length difference between the two optical fibers 74a, 74b. This conveniently enables the creation of two optical pulses that track each other identically.

Another fiber coupler and pair of optical fibers can be added to create four identical optical pulses separated from each other in time. In particular, the optical arrangement further includes a second 1×2 fiber splitter 72b to which the single optical fiber 74c carrying the two identical and temporally spaced optical pulses P1, P2 is coupled. Thus, the two identical optical pulses P1, P2 are input into the second 1×2 fiber splitter 72b and split into four identical optical pulses P1a, P1b, P2a, P2b (i.e., the optical pulse P1 is split into optical pulses P1a, P1b, and the optical pulse P2 is split into optical pulses P2a, P2b). Two optical fibers 74d, 74e of different optical lengths are connected to the respective outputs of the second 1×2 fiber splitter 72b, such that the two sets of two identical optical pulses P1a, P1b and P2a, P2b respectively propagate within the two optical fibers 72d, 72e. The optical arrangement further includes a second 2×1 fiber coupler 76b into which the two sets of identical optical pulses P1a, P1b and P2a, P2b are input and combined, and output to a single optical fiber 74f. By making the lengths of the optical fibers 74d, 74e different from each other, the two optical pulses input into the second 1×2 fiber splitter 72b are effectively split into four identical optical pulses that propagate through the single optical fiber 74f and spaced out by a time difference determined by the optical path length difference between the two optical fibers 74d, 74e. This conveniently enables the creation of four optical pulses that track each other identically.

Referring back to FIG. 1, once the camera 28 acquires the data voxel by storing all spatial component values of each of the four interference pulses Iₐ-I_{d} within the four bins 70 of each of the detectors 68, these data can be sent to the processor 30 (which can, e.g., take the form of a computer, field-programmable gate array or application specific integrated circuit), which is configured for determining a physiologically-dependent optical parameter (e.g., absorption) of the target voxel 14 based on the four values stored in the data storing bins 70 of each detector 68. As briefly discussed above, the four spatial component values can be power values Pₐ-P_{d}, which can be used by the processor 30 to reconstruct the amplitude of the signal light 44, and thus, can be said to be representative of the physiologically-dependent optical parameters (e.g., optical absorption) of the target voxel 14.

The spatial component power values Pₐ-P_{d} for all four interference light patterns Iₐ-I_{d} can be used in accordance with known “quadrature detection” methods to reconstruct the amplitude of the signal light 44, which is proportional to the number of tagged photons emerging from the target voxel 14 (i.e., the number of photons in the signal light 44), and thus, can be used to measure optical absorption in the target voxel 14 (e.g., for the purpose of measuring spatially localized neural activity-correlated changes in the level of deoxygenated and/or oxygenated hemoglobin concentration or relative abundance in the brain, which appear as localized changes in the optical absorption of blood). In the illustrated embodiment, it should be understood that because of the diffusive scattering of light over large distances through the brain and skull, the interference light pattern 48 detected by the lock-in camera 28 takes the form of a random speckle pattern in which each localized speckle grain has a definite, but random phase offset in the interference light pattern 48 (i.e., a beat pattern) between the reference light 42 and the signal light 44. This results in the unknown random phases in the beat patterns measured by each detector 68 (or pixel) in the equations set forth below.

In particular, assuming that the sample light 40 has a rectangular-shaped pulsed waveform, the power detected at a single detector 68 (or pixel) for each optical pulse at one of the four phases can be expressed as:

Value_{1,k}=P_{background}+P_{signal}+P_{reference}+2(P_{signal}×P_{reference})^{1/2}×cos(φ_{control}−φ_{unknown1,speckle k})+2(P_{signal}×P_{background})^{1/2}×cos(2π×fᵤₛ−φ_{unknown2,speckle k})+2(P_{reference}×P_{background})×cos(2π×fᵤₛ−φ_{unknown3,speckle k}), [1]

where P_{background} represents light at frequency f−fᵤₛ that has not been tagged with the ultrasound 32; P_{signal} represents light at frequency f that has been tagged with the ultrasound 32; P_{reference} represents the reference light at frequency f; φ_{control} is a control phase shift introduced into the reference light 42 for each detected interference pattern 48; (P_{unknown1, speckle k}, φ_{unknown2, speckle k}, and φ_{unknown3, speckle k} are random phases at the kth speckle grain at the time of measurement, which originate via multiple scattering of coherent light inside the tissue.

The terms P_{background}+P_{signal}+P_{reference} are constant across all four optical pulses with different control phase values φ_{control}. The terms 2(P_{signal}×P_{background})^{1/2}×cos(2π×fᵤₛ−φ_{unknown2})+2(P_{reference}×P_{background})^{1/2}×cos(2π×fᵤₛ (P_{unknown3}) oscillate at the frequency fᵤₛ, and are not detected by the lock-in camera 28 given appropriate light pulse duration and integration time, and thus, can be ignored. As such, equation [1] can be reduced to:

Valueₖ=P_{background}+P_{signal}+P_{reference}+2(P_{signal}×P_{reference})×cos(φ_{control}−φ_{unknown}), [2]

which is analogous to:

A+B×Cos(φ_{control}+φ_{unknown}), [3]

where φ_{control} can be respectively set to 0, π/2, π, and 3π/2 to create four equations. Both the amplitude B and the unknown phase φ_{unknown} can be extracted by solving the resulting four equations using the standard trigonometric identities.

Thus, the term magnitude of P_{signal}×P_{reference} can be extracted by shifting the control phase φ_{control} successively on each of four successive pulses φ_{control}=0, π/2, π, and 3π/2. Even though φ_{unknown} is an unknown and random phase, specific to each pixel, which results from the laser speckle pattern due to light scattering in the tissue, by measuring and storing each of these four measurements at different control phase values φ_{control}, the value of the interference term 2(P_{signal}×P_{reference})^{1/2} may be extracted via the known principal of “quadrature detection.” Because the power of the reference light P_{reference} is known or independently measurable, the interference term 2(P_{signal}×P_{reference})^{1/2} serves as a measurement of the power of the signal light P_{signal}. Thus, using a known scaling relationship, the power of the signal light P_{signal} can be determined (either in the absolute sense or relative sense) from the extracted term interference term 2(P_{signal}×P_{reference})^{1/2}.

Because the speckle phases are random, according to the known principles of parallel speckle detection in UOT or in wavefront measurement from strongly scattering media, it is known that a single-pixel detector will not scale to high signal to noise ratios. In particular, the aggregate signal over a large single-pixel detector would scale as the square root of detector size, but so would shot noise in the background, and hence the signal to noise ratio performance of a large detector would not increase with detector size. In contrast, as described in the equations below, with lock-in detection at each detector (or pixel), the aggregate signal scales linearly with the number of pixels, while the aggregate background shot noise scales as the square root, and hence signal to noise performance increases as the square root of the number of pixels, giving a strong advantage for using large numbers of pixels.

It can be assumed that the amplitude of P_{reference} is much greater than the amplitude of P_{background}, and the amplitude of P_{signal} is naturally much less than the amplitude of P_{reference}, since the ultrasound tagged signal light 44 originates from a very small target voxel 14 within the tissue and the tagging efficiency (i.e., the number of tagged photons relative to a number of untagged photons scattered by the target voxel 14) within that target voxel 14 is a small fraction. Thus, only interference terms containing P_{reference} are significant in the sum representing the intensity measured by each pixel (i.e., P_{background} P_{signal}+P_{reference}+2(P_{signal}×P_{reference})^{1/2}×cos(φ_{control}−φ_{unknown1})).

Therefore, the dominant signal source contributing to detection has the following number of photons impinging on one pixel:

dominant signal=(ε/hv)×2(P_{signal}×P_{reference})^{1/2}τ; [4]

and the dominant noise source in the quadrature measurement of this amplitude is due to the shot noise in the reference light 42, and has the following number of photons impinging on each pixel:

dominant noise=((ε/hv)×P_{reference}×τ)^{1/2}; [5]

where ε is a detector efficiency scaling factor, P is the power for each of the ultrasound tagged photons, hv is the per-photon energy (with h as Plank's constant, and v as the frequency of the light), and τ is the integrated pulse widths used in the measurement.

With a number of pixels N, the signal-to-noise ratio (SNR) scales with N^{1/2}, since the total shot noise grows as N^{1/2}, whereas the total signal grows as N, so that:

SNR_{N pixels}=(N×(ε/hv)×τ×P_{signal})^{1/2}, [6]

which shows that the SNR improves with increasing number of pixels in the lock-in camera 28. Thus, the Poisson nature of photon shot noise statistics is being utilized to determine the fundamental signal to noise ratio.

It should be appreciated that although the UOT system 10 has been described as using a 4-bin quadrature detection scheme to reconstruct the amplitude of the signal light 44 from the interference light patterns 48, and therefore, utilizes four data storing bins 70 (and four optical pulses) for each detector 68 (or pixel) of the lock-in camera 28 to store the intensity values of the respective four interference patterns 48 over the four different phases, the UOT system 10 may utilize less than four phases (e.g., three phases equal to 0, 2π/3, and 4π/3), or may even utilize two phases (e.g., 0 and π) to reconstruct the amplitude of the signal light 44 from the interference light patterns 48, and therefore utilizes three data storing bins 70 (and three optical pulses) or only two data storing bins 70 (and only two optical pulses) for each detector 68 (or pixel) to store the intensity values of the respective interference patterns 48 over the phases. It should further be appreciated that although the phases of the 4-bin quadrature scheme, as well as the three-bin and two-bin detection schemes, have been described as being equally spaced, the phases used in any of these detection schemes can be unequally spaced. For example, for the three-bin detection scheme, the phases can be selected to be 0, π, and 4π/3, or for a two-bin detection scheme, the phases can be selected to be 0 and 4π/3.

In the case of a two-bin detection scheme, rather than obtaining a quadrature amplitude from each pixel 68, the power of the signal light 44 can be computed as the absolute difference between the two intensity values stored in the two data storing bins 70 for each pixel 68 and then averaged.

In particular, assuming that the sample light 40 has a rectangular-shaped pulsed waveform, the power detected at a single detector 68 (or pixel) for each optical pulse at one of the two phases can computed by expanding equation [2] for two phases of 0 and 180 degrees, as follows:

Value_{l,k}=P_{background}+P_{signal}+P_{reference}+2(P_{signal}×P_{reference})^{1/2}×cos(0−φ_{unknown}); [6]

and

Value_{2,k}=P_{background}+P_{signal}+P_{reference}+2(P_{signal}×P_{reference})^{1/2}×cos(π−φ_{unknown}), [7]

where Value₁ is the intensity value in the first data storing bin 70 of the respective pixel k, and Value₂ is the intensity value in the second data storing bin 70 of the respective pixel k.

It is noted that the term P_{background}+P_{signal}+P_{reference} in each of equations [6] and [7] represents the DC terms of the two interference light patterns, whereas the terms 2(P_{signal}×P_{reference})^{1/2}×cos(φ_{control}−φ_{unknown}) and 2(P_{signal}×P_{reference})^{1/2}×cos(φ_{control}−φ_{unknown}+π) in the respective equations [6] and [7] represent the AC beat patterns of the interference light patterns 48, which differ in phase by 180 degrees, such that they sum to zero.

The terms P_{background}+P_{signal}+P_{reference} are constant across the two phases, and thus, can be eliminated by taking the difference of the intensity values Value_{1,k} and Value_{2,k}, leaving only the difference of the interference terms, as 2(P_{signal}×P_{reference})^{1/2}×cos( _{control}−φ_{unknown})−2(P_{signal}×P_{reference})^{1/2}×cos(φ_{control}−φ_{unknown}+π), which can be summed over all of the k pixels and then averaged in accordance with the following equation:

2(P_{signal}×P_{reference})^{1/2}(Σk|Value_{1,k}−Value_{2,k}|)/N across all k=1,2, . . . N speckles or pixels, [8]

the average across all pixels of the absolute value of the difference across corresponding pixels. Based on known scaling relationship between the power of the reference light P_{reference} and the power of the signal light P_{signal} discussed above, the power of the P_{signal} can be expressed as:

P_{signal}∝(Σk|Value_{1,k}−Value_{2,k}|)/N, [9]

i.e., the average across all pixels of the absolute value of the difference across corresponding pixels.

It should be noted that sign-rectifying operations of the intensity value differences other than taking the absolute can be utilized to obtain the power of the signal light 44. For example, a squaring operation can be applied to the intensity value differences. Thus, the power of the signal light 44 can alternatively be computed as the square of the difference between the two intensity values stored in the two data storing bins 70 for each pixel 68 and then averaged in accordance with the following equation:

P_{signal}∝(Σk(Value_{1,k}−Value_{2,k})²)/N [10]

across all k=1,2, . . . N speckles or pixels.

Thus, it can be appreciated that the intention of the two-bin measurement is to arrive at a precise estimate of power of the signal light P_{signal} up to the aforementioned scaling relationship defined by the strength of the reference light P_{reference}, by removing the terms that are constant between the two measurements of the lock-in camera 28, and removing the unknown speckle-specific phases, and instead extracting only the amplitude of the cosine term. In the context of the UOT, just as with the quadrature detection scheme described above, the two-bin detection scheme serves as a measurement of light absorption at a single spatial voxel within the tissue. However, the two-bin detection scheme represents a simplification that leads to only a small constant decrease factor in the signal to noise ratio. The dominant signal source contributing to detection has the following number of photons impinging on one pixel:

dominant signal=(ε/hv)×B×τ/2×|cos(0+φ)−cos(π+φ)|_{average over φ in [0,2π]}; [11]

and the dominant noise source in the two-bin measurement of this amplitude is due to the shot noise in the reference light 42, and has the following number of photons impinging on one pixel:

dominant noise=(ετ/hv)^{1/2}×(2/π)B/A^{1/2}, [12]

where A and B are constants in the equation A+B(cos φ), B is proportional to the number of tagged photons per detector 68, ε is a detector efficiency scaling factor, hv is the per-photon energy (with h as Plank's constant, and v as the frequency of the light), and τ is the integrated pulse widths used in the measurement, and φ is a random, pixel-specific speckle phase.

With a number of pixels N, the signal-to-noise ratio (SNR) scales with N^{1/2}, since the total shot noise grows as N^{1/2}, whereas the total signal grows as N, so that:

SNR_{N pixels}=(N×(ετ/hv)^{1/2}×(2/π)×B/A^{1/2}, [13]

which shows that, just as in the quadrature detection scheme, the SNR improves with increasing number of pixels in the lock-in camera 28, and the Poisson nature of photon shot noise statistics is being utilized to determine the fundamental signal to noise ratio.

Notably, the use of a two-bin detection scheme, rather than the four-bin quadrature scheme, provides the advantage that only two optical pulses, as opposed to four optical pulses, needs to be generated, thereby shortening the time period needed to take a measurement of the target voxel 14, and thus, alleviating the speckle decorrelation time limitation.

Although the UOT system 10 has been described as computing the intensity values Valueₖ of the speckle grains of each interference light pattern 48 in order to obtain the power of the light signal P_{signal}, it should be appreciated that the UOT system 10 may alternatively or optionally compute the random phases φ_{unknown} of the kth speckle grains of each interference light pattern 48 in order to obtain the phase across the wavefront of the light signal.

For example, in an optional embodiment, a digital optical phase conjugation (DOPC) technique can be used to boost the sensitivity of the pulsed UOT detection. DOPC can be performed in the context of schemes that rely on time reversal based optical phase conjugation using “guidestars” localized in three dimensions, for instance, using schemes, such as Time Reversal of Ultrasound-Encoded Light (TRUE) (see, e.g., Xu X, Liu H., Wang L V, “Time-Reversed Ultrasonically Encoded Optical Focusing into Scattering Media,” Nature Photonics, Vol. 5, No. 3, pp. 154-157 (Mar. 1, 2011); Wang Y M, Judkewitz B, DiMarzio C A, Yang C., “Deep-Tissue Focal Fluorescence Imaging with Digitally Time-Reversed Ultrasound-Encoded Light,” Nature Communications, Vol. 3, Article 928 (Jun. 16 2012); Horstmeyer R., Ruan H, Yang C, “Guidestar-Assisted Wavefront-Shaping Methods for Focusing Light into Biological Tissue,” Nature Photonics, Vol. 9, No. 9, pp. 563-571 (Sep. 1, 2015).

These methods are used to focus light to a guide-star-defined point deep inside a scattering medium, by measuring the wavefront emanating from the guidestar and digitally time-reversing (e.g., phase conjugating) light in order to cause the light to “play back” its path through the scattering medium and come to focus at the guidestar position. In the context of UOT, the guidestar is the focal point of an ultrasound beam. In these methods, the phase of a tagged light field originating from a given three-dimensional guidestar voxel in the brain is measured using demodulation and quadrature detection, and then an approximate phase-conjugate, i.e., approximate time-reverse light field, possibly amplified in intensity, is “played back” to focus light to the three-dimensional guidestar location despite the effects of strong or even diffusive scattering in the tissue.

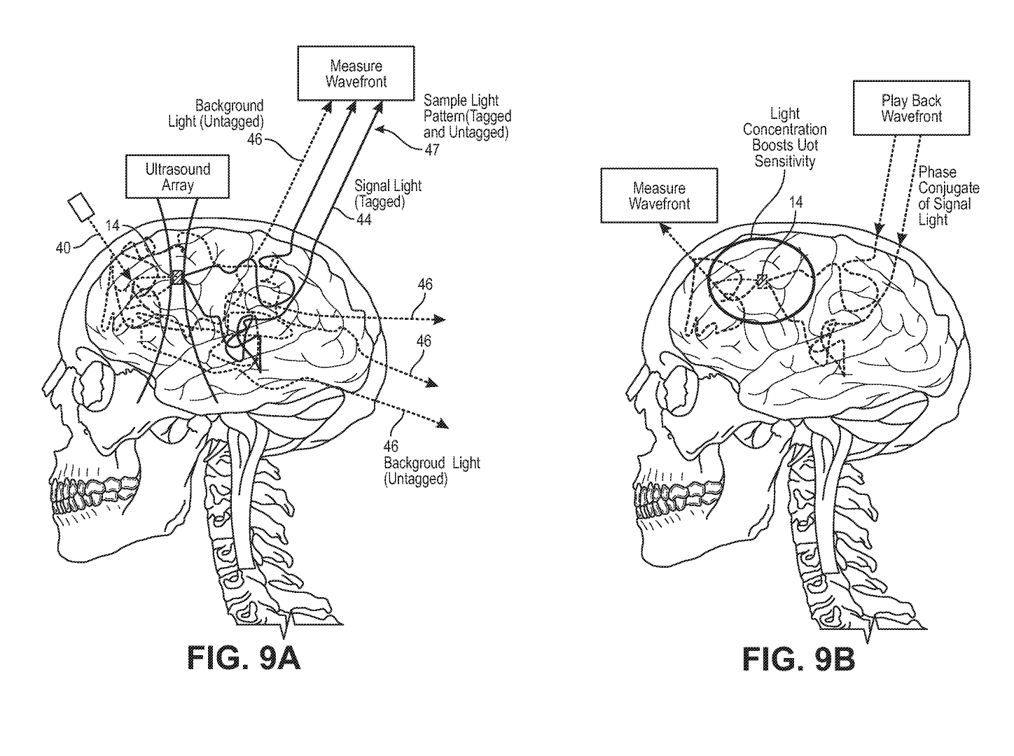

In the context of the UOT system 10 described herein, the phase of the wavefront of the signal light 44 originating from the target voxel 14 (i.e., the guidestar) is measured using the pulsed UOT detection scheme described above, as illustrated in FIG. 9a, with the exception that, in addition to extracting the power of the wavefront of the P_{signal}, the unknown phase φ_{unknown} of the wavefront of the signal light 44 is extracted using the known principles of quadrature detection. As illustrated in FIG. 9b, the wavefront of the signal light 44 is then amplified and retransmitted back in the opposite direction and focused onto the target voxel 14 (i.e., the guidestar), where it is tagged by the ultrasound 32 and may then be detected by the same or another lock-in camera so as to perform the process iteratively. The retransmission should be timed such that the light through the voxel is coincident in time with an ultrasound pulse passing through the voxel.

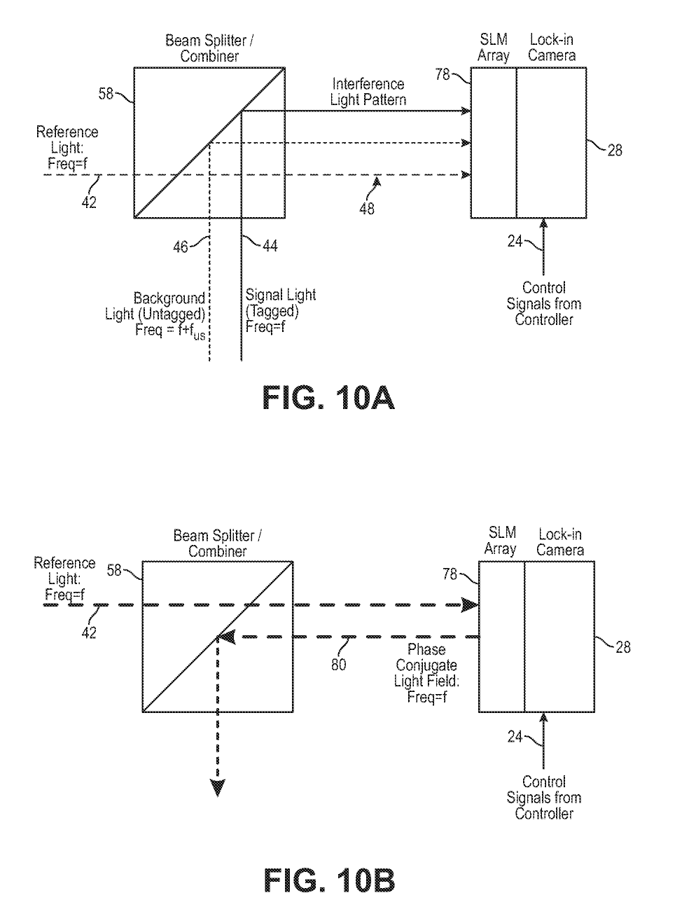

Referring to FIG. 10a, the phase of the signal light 44 extracted in the pulsed UOT detection scheme may be used in conjunction with a spatial light modulator (SLM) array 78 that is co-registered (e.g., pixel-by-pixel) with the lock-in camera 28 to perform optical phase conjugation of the signal light 44 (see, e.g., Laforest T, Verdant A, Dupret A, Gigan S., Ramaz F, Tessier G, “Co-Integration of a Smart CMOS Image Sensor and a Spatial Light Modulator for Real-Time Optical Phase Modulation,” Proc. Of SPIE-IS&T, Vol. 2014, 9022:90220N-1 (March 2014).

The SLM array 78 may include any of a number of different amplitude and/or phase modulator structures, such as liquid crystals, re-positionable microelectromechanical systems (MEMS) mirrors, ferroelectrics, digital micro-mirror device pixels, among others. In one embodiment, the SLM array 78 may be semi-transparent (e.g., a liquid crystal modulator backed by a partial reflector), and can be inserted into the light path between the entry of the reference light 42 and the lock-in camera 28. The SLM array 78 may be built directly on top of the lock-in camera 28 to create a phase conjugation array, with this arrangement being similar to the pulsed UOT detection scheme described above.

Referring to FIG. 10b, post-detection, each pixel 68 of the lock-in camera 28 will send the conjugate phase information to the SLM array 78 (conjugate phase information being the negative of the detected phase). Each pixel may have internal electronics (e.g., transistors) that compute and sends the desired phase adjustment as an electrical voltage to adjust the corresponding pixel phase or amplitude of the SLM array 78. In a phase-only optical phase conjugation scheme, each pixel of the phase conjugation array will simply “actuate” the conjugate phase, such that light reflected from the pixel will accrue the phase. Amplitude-only phase conjugation can alternatively be performed by reflecting or not reflecting the input light based on the conjugate phase information. In aggregate, the same reference light 42 used in the detection process (while blocking the sample light 40) or light precisely aligned with the reference light 42 will reflect off the phase conjugation array to create a phase conjugate light field 80 that will focus back to the target voxel 14.

The improvement in contrast of this return light 80 (i.e., the phase conjugate light field) to the target voxel 14 is given by: Contrast A=α*((N−1)/M+1), wherein N is the number of input optical modes (or the number of photons if less than the number of input optical modes), which is approximately equal to the number of pixels on the phase conjugation array); M is the number of target optical modes in the target voxel 14, and α equals 1 when a full phase and amplitude conjugation is performed, and is some value smaller than 1 when a phase only, amplitude only, and/or coarse grain phase conjugation is performed. The term “coarse grain,” in this context, means that the phase playback at each pixel can take only a finite number of possible values.

The phase conjugation process can be iterated many times, each time taking the light field, resulting from the last step, and phase conjugating that scattered light field. The contrast improvement can be expected to grow as (contrast A)^{K}, where K is the number of iterations. Thus, the number of photons traveling through the target voxel 14 can be exponentially amplified, thereby improving the effective modulation depth of the UOT (i.e., the fraction of the ultrasound tagged photons reaching the detector). The addition of phase conjugation to the UOT system 10 could be used to increase the number of collected tagged photons, increase modulation depth, or decrease ultrasound intensity or duty cycle requirements.

Besides detecting random phases φ_{unknown} of the kth speckle grains of each interference light pattern 48 in order to obtain the phase across the wavefront of the light signal for purposes of performing DOPC, the UOT system 10 may detect random phases m unknown of the kth speckle grains of each interference light pattern 48 for other purposes, such as detecting faster signals due to light scattering, as opposed to light absorption changes. For example, the phase of each kth speckle grain of signal light may be determined using quadrature detection (i.e., four bins) in accordance with the equation:

φ=arctan(Value_{1,k}−Value_{3,k})/(Value_{2,k}−Value_{4,k}), [14]

where Value_{1,k} is the intensity of the kth speckle grain of the interference light pattern 48a at a phase of 0, Value_{2,k} is the intensity of the kth speckle grain of the interference light pattern 48b at a phase of π/2, Value_{3,k} is the intensity of the kth speckle grain of the interference light pattern 48c at a phase of π, and Value_{4,k} is the intensity of the kth speckle grain of the interference light pattern 48d at a phase of 3π/2.

Alternatively, the phase of each kth speckle grain of signal light may be determined using other phase estimation techniques. For example, a discretized phase of each kth speckle grain of signal light may be determined, e.g., in the range of π/2 to 3π/2 versus −π/2 to −3π/2. In any event, the phases of the wavefront of the signal light can be repeated to determine temporal changes in the wavefront phases, and the magnitude of such temporal phase changes can be computed over the kth speckle grains of the signal light. In this manner, the UOT system 10 may be sensitive to changing optical phases in the target voxel 14 due to optical scatter changes, which are indicative of neural activity.

Performance estimates for the UOT system 10 described herein in the detection of a blood-oxygen-level dependent signal in the brain through the skull as a function of the number of pixels in the lock-in camera 28 used (in this case, 10 million pixels or higher) indicate that neural activity dependent changes in the blood-oxygen-level dependent signal could be detected at hundreds to thousands of voxels per 100 millisecond temporal sample. In this calculation, the use of a 2 MHz ultrasound, and thus a spatial resolution on the order of ½ millimeter, is assumed, exceeding the spatial resolution of traditional blood-oxygen-level dependent signal measurements, like functional MRI (fMRI), and vastly exceeding the many millimeter to multiple centimeter-scale spatial resolution of diffuse optical tomography, including time-gated forms of diffuse optical tomography. In this calculation, it is further assumed that millions of tagged photons must be collected from the target voxel 14 per temporal sample in order to measure naturally occurring blood-oxygen-level dependent signals functional changes in the human brain, which are on the order of small fractions of a percent, while overcoming shot noise fluctuations in the number of detected tagged photons.

In one embodiment, the processor 30 utilizes blood-oxygen-level dependent signals detected by the lock-in camera 28 to determine the neural activity in the brain; that is, blood-oxygen-level dependent signals provide a sense of the level of deoxygenated and/or oxygenated hemoglobin concentration or relative abundance in the target voxel 14 in the brain, and given the known coupling between cerebral hemodynamics and neuronal activity, the processor 30 can thereby determine the extent of neuronal activity in that target voxel 14. In another embodiment, the UOT system 10 detects blood-oxygen-level dependent signals over multiple wavelengths of the sample light, in which case, the processor 30 may determine and compare the optical absorption characteristics of the target voxel 14 of blood-oxygen-level dependent signals over the different wavelengths of sample light in order to determine the level of deoxygenated and/or oxygenated hemoglobin concentration or relative abundance present in the target voxel 14 according to known principles of functional infrared spectroscopy, for instance by solving two equations in two unknowns relating the measured absorption at two wavelengths to the level of deoxygenated and/or oxygenated hemoglobin concentration or relative abundance in the blood, or alternatively several equations in several unknowns representing absorption at several wavelengths in order to determine the concentrations of several molecular species in the target voxel 14.

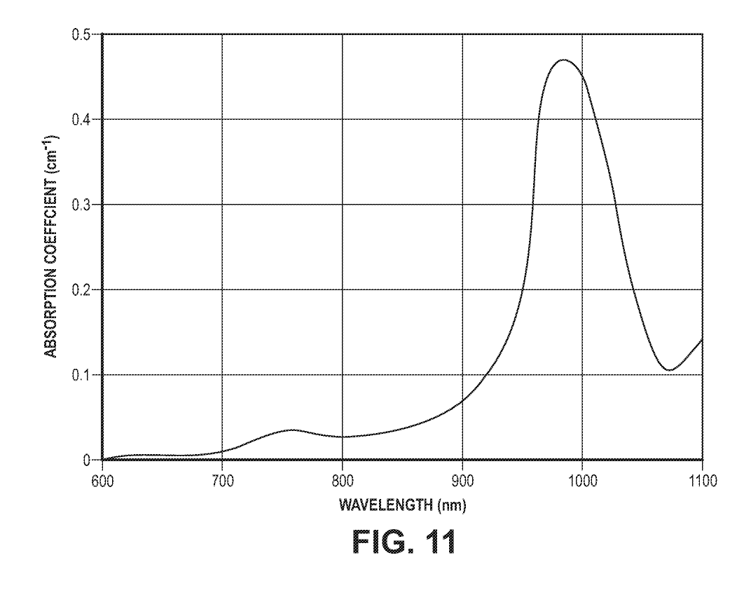

In one particularly advantageous embodiment, instead of detecting blood-oxygen-level dependent signals, the processor 30 may detect faster signals of neuronal activity, such as in the brain, to determine the extent of neuronal activity in the target voxel 14. Neuronal activity generates fast changes in optical properties, called “fast signals,” which have a latency of about 10-100 milliseconds and are much faster than the metabolic (approximately 100-1000 milliseconds) and hemodynamic (hundreds of milliseconds to seconds) evoked responses (see Franceschini, M A and Boas, D A, “Noninvasive Measurement of Neuronal Activity with Near-Infrared Optical Imaging,” Neuroimage, Vol. 21, No. 1, pp. 372-386 (January 2004)). Additionally, is believed that brain matter (e.g., neurons and the extracellular matrix around neurons) hydrates and dehydrates as neurons fire (due to ion transport in and out of the neurons), which could be measured via determining the absorption characteristics of water in the target voxel 14. In this case, it is preferred that the target voxel 14 be minimized as much as possible by selecting the appropriate ultrasound frequency (e.g., two to six times the size of a neuron, approximately 100 micrometers) in order to maximize sensitivity to highly localized changes in fast indicators of neural activity. As illustrated in FIG. 11, the optical absorption coefficient of water is relatively high for wavelengths of light in the range of 950 nm-1080 nm. Thus, for maximum sensitivity to changes in optical absorption of tissue due to changes in the level of water concentration or relative water concentration in the brain matter, it is preferred the wavelength of the sample light be in the range of 950 nm-1080 nm.

Regardless of the nature of the detected signal and physiologically-dependent optical parameter, the processor 30 may optionally use a computational model of light propagation in the tissue, and deconvolution or inverse problem optimization methods/algorithms, to improve the spatial resolution of the resulting measurement. Empirical measurements of a sample may be compared to those predicted by a model of the spatial layout of absorbers of the sample incorporating an effective point spread function of detection, such that the model may be improved to obtain an optimal match between the model predictions and the observed signals from the sample (see Powell S., Srridge S R, Leung T S, “Gradient-Based Quantitative Image Reconstruction in Ultrasound-Modulated Optical Tomography: First Harmonic Measurement Type in a Linearized Diffusion Formulation,” IEEE Transactions on Medical Imaging, Vol. 35, No. 2, pp. 456-467 (February 2016).

Although the UOT system 10 has been described herein as acquiring only one measurement of the target voxel 14, it should be appreciated that the UOT system 10 may acquire multiple measurements of the target voxel 14 over time that yields a time trace indicative of time varying physiologically depending optical properties in the target voxel 14, such as time-varying optical absorption in the target voxel 14 due to functional changes in the brain. Optionally, two time traces of the target voxel 14 can be acquired, one time trace being generated with the ultrasound 32 turned on at regular intervals in the same manner described above, and another time trace generated with the ultrasound 32 turned off at regular intervals. For example, a measurement of the target voxel 14 may be acquired when the ultrasound 32 turned on to create a first data point on the first time trace; a measurement of the target voxel 14 may be acquired when the ultrasound 32 is turned off to create a first data point on the second time trace; a measurement of the target voxel 14 may be acquired when the ultrasound 32 is turned on to create a second data point on the first time trace; a measurement of the target voxel 14 may be acquired when the ultrasound 32 is turned off to create a second data point on the second time trace; and so forth. The second time trace may provide a baseline null signal measurement trace, which is useful for tracking secondary variations distinct from the first time trace's signal variations due to the ultrasound 32.

Although the controller 38 and processor 40 are described herein as being separate components, it should be appreciated that portions or all functionality of the controller 24 and processor 26 may be performed by a single computing device. Furthermore, although all of the functionality of the controller 24 is described herein as being performed by a single device, and likewise all of the functionality of the processor 26 is described herein as being performed by a single device, such functionality each of the controller 24 and the processor 26 may be distributed amongst several computing devices. Moreover, it should be appreciated that those skilled in the art are familiar with the terms “controller” and “processor,” and that they may be implemented in software, firmware, hardware, or any suitable combination thereof.

Referring now to FIG. 12, the physical implementation of the UOT system 10 will be described. As there shown, the UOT system 10 includes a wearable unit 90 that is configured for being applied to the user 18, and in this case, worn on the head of the user 18, and an auxiliary head-worn or not head-worn unit 92 coupled to the wearable unit 90 via a wired connection 94 (e.g., electrical wires). Alternatively, the UOT system 10 may use a non-wired connection (e.g., wireless radio frequency (RF) signals) for providing power to or communicating between components of the respective wearable unit 90 and auxiliary unit 92.

In the illustrated embodiment, the wearable unit 90 includes a support structure 94 that either contains or carries the transducer arrangement 34 of the acoustic assembly 20 (shown in FIG. 2), the interferometer 22, and the lock-in camera 28. The wearable unit 90 may also include an output port 98a from which the ultrasound 32 generated by the acoustic assembly 20 (shown in FIG. 1) is emitted, an output port 98b from which the sample light 40 generated by the interferometer 22 (shown in FIG. 1) is emitted, and an input port 98c into which the sample light pattern 47 comprising the tagged signal light and untagged background light are input into the interferometer 22. It should be appreciated that although the input port 98c is illustrated in close proximity to the input ports 98a, 98b, the proximity between the input port 98c and the output ports 98a, 98b may be any suitable distance. The support structure 94 may be shaped, e.g., have a banana, headband or hat shape, such that the ports 98 are in close contact with the outer skin of the body part, and in this case, the scalp of the head 16 of the user 18. An index matching fluid maybe used to reduce reflection of the light generated by the light source 50 of the interferometer 22 from the outer skin, and an ultrasound transmitting adhesive or acoustic coupling material can be used to facilitate conduction of the ultrasound 32 into the body part. An adhesive or belt (not shown) can be used to secure the support structure 94 to the body part.

The auxiliary unit 92 includes a housing 96 that contains the controller 24 and the processor 30 (shown in FIG. 1). The auxiliary unit 92 may additionally include a power supply (which if head-worn, may take the form of a rechargeable or non-chargeable battery), a control panel with input/output functions, a display, and memory. The auxiliary unit 92 may further include the signal generator 36 of the acoustic assembly 20, as well as any drive circuitry used to operate the interferometer 22.

The interferometer 22 and lock-in camera 28 are preferably mechanically and electrically isolated from the acoustic assembly 20, such that the emission of the ultrasound 32 by the acoustic assembly 20, as well as the generation of RF and other electronic signals by the acoustic assembly 20 minimally affects the detection of the optical signals by the interferometer 22 and generation of data by the lock-in camera 28. The wearable unit 90 may include shielding (not shown) to prevent electrical interference and appropriate materials that attenuate the propagation of acoustic waves through the support structure 94.

Having described the arrangement and function of the UOT system 10, one method of operating the UOT system on a user will now be described. In this method, ultrasound 32 is delivered into the target voxel 14 in the anatomical structure 16, and sample light 40 is delivered into the anatomical structure 16, wherein a portion 40a of the sample light 40 passing through the target voxel 14 is scattered by the anatomical structure 16 as the signal light 44, and another portion 40b of the sample light 40 not passing through the target voxel 14 is scattered by the anatomical structure 16 as background light 46 that combines with the signal light 44 to create the sample light pattern 47. As exemplified above, the anatomical structure 16 may be an intact head comprising the scalp, skull, and brain matter. Due to the high resolution of the UOT system 10, the target voxel 14 may be smaller than one mm³.

The reference light 42 is combined with the sample light pattern 47 to generate an interference light pattern 48 (e.g., in a homodyne manner), and in this method, a speckle light pattern. The ultrasound 32 and sample light 40 are pulsed in synchrony at the target voxel 14, such that only the signal light 44 is shifted (i.e., tagged) by the ultrasound 32. That is, as described above, each pulse of the sample light 40 will pass through the target voxel 14 only as the ultrasound 32 passes through the target voxel 14, such that no portion of the background light 46 will be tagged by the ultrasound 32. The interference light pattern 48 is sequentially modulated to generate a plurality of different interference light patterns 48. The spatial components of any particular interference light pattern 48 can then be simultaneously detected, and a plurality of values can be stored in the respective data storing bins 70 (either in bins 70a, in bins 70b, in bins 70c, or bins 70d) of the detectors 68. The values are representative of the spatial component for the respective interference light pattern 48. The physiologically-dependent optical parameter of the target voxel 14 is then determined based on the spatial component values stored in the data storing bins 70. Due to the high speed of the lock-in camera 28, the spatial components for any particular interference light pattern 48 may be simultaneously detected and stored in the respective data storing bins 70 very quickly. For example, in one embodiment, the spatial components for any particular interference light pattern 48 may be simultaneously detected, and the resulting spatial component values for all the interference light patterns 48 are stored in the respective data storing bins 70 within 1 millisecond. In another embodiment, the spatial components for any particular interference light pattern 48 may be simultaneously detected, and the resulting spatial component values for all the interference light patterns 48 are stored in the respective data storing bins 70 within 1 microsecond to 1 millisecond.

Although the UOT system 10 has been described as non-invasively measuring one target voxel 14 at a time, the UOT system 10 may be modified to measure multiple target voxels 14 at a time. For example, in one particular embodiment, an acoustic assembly may simultaneously generate multiple foci in parallel (i.e., focuses the ultrasound on multiple target voxels at the same time), with each ultrasound focus being modulated at a different optical frequency (e.g., f₁−fᵤₛ, f₂−fᵤₛ, f₃−fᵤₛ, etc.), with separate frequency-shifted reference light (e.g., f₁, f₂, f₃, etc.) being used to interfere with the resulting signal light in a homodyne manner. Separate lock-in cameras may be utilized to detect the different interference patterns corresponding to the different target voxels.

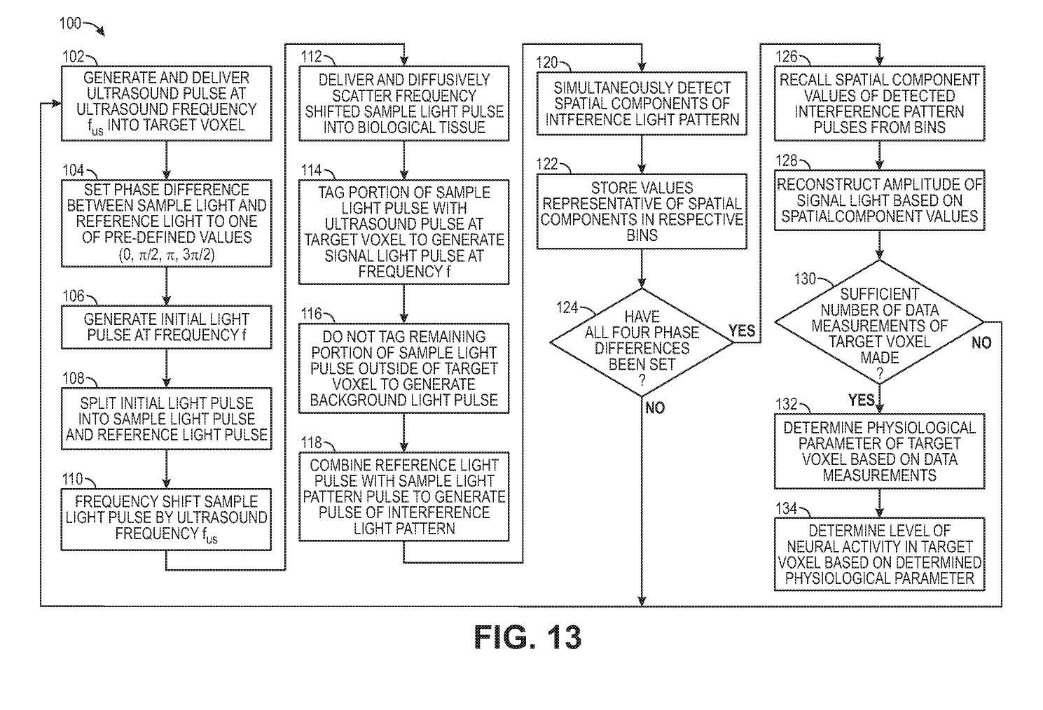

Referring to FIG. 13, one particular method 100 performed by the UOT system 10 to non-invasively measure the target voxel 14 in anatomical structure 16 will now be described. This particular method 100 implements the pulsing sequence of FIG. 5.

The controller 24 operates the acoustic assembly 20 to generate and deliver a pulse of ultrasound 32 having a frequency fᵤₛ (initially, ultrasound pulse Uₐ illustrated in FIG. 5) into the anatomical structure 16, e.g., by sending a control signal to the signal generator 36 to pulse an electrical signal on and off (step 102). The controller 24 sets the phase difference between the sample light 40 and the reference light 42 to one of the four pre-defined values (0, π/2, π, and 3π/2) by sending a control signal to the phase shifter 54 of the interferometer 22 (step 104). This pre-defined phase difference value may be first set to 0. Next, the controller 24 operates the interferometer 22 to generate and emit a pulse of source light 38 having a frequency f, e.g., by sending a control signal to the drive circuit to pulse the light source 50 on and off (step 106). The interferometer 22 (e.g., via the beam splitter 52) splits the pulse of source light 38 into a pulse of sample light 40 (initially, the sample light pulse Lₐ illustrated in FIG. 5) and a pulse of reference light 42 (step 108).

The wavelength (and thus, the frequency f) of the source light 38 may be selected based on the physiologically-dependent optical parameter to be ultimately determined. For example, if the physiologically-dependent optical parameter is the level of deoxygenated and/or oxygenated hemoglobin concentration or relative abundance, the wavelength of the source light 38 may be in the range of 605 nanometers to 950 nanometers, whereas if the physiologically-dependent optical parameter to be determined is a water absorption level (level of water concentration or relative water concentration), the wavelength of the source light 38 may be in the range of 950-1080 nanometers.

Next, prior to the pulse of sample light 40 entering the anatomical structure 16, the controller 24 operates the interferometer 22 to frequency shift the pulse of sample light 40 by the ultrasound frequency fᵤₛ, e.g., by sending a control signal to the frequency shifter 56, resulting in the pulse of sample light 40 having a frequency f−fᵤₛ (step 110). It should be appreciated that, although this frequency shifting technique implements the frequency shifting technique illustrated in FIG. 3a, other frequency shifting techniques can be utilized, such as those illustrated in FIGS. 3b-3d. The frequency-shifted pulse of sample light 40 is then delivered into and diffusively scattered within the anatomical structure 16 (step 112). As the pulse of frequency shifted sample light 40 scatters diffusively through the anatomical structure 16, a portion will pass through the target voxel 14 and be frequency shifted (i.e., tagged) back to its original frequency f by the pulse of ultrasound 32 passing through the target voxel 14, resulting in a pulse of scattered signal light 44 having the same frequency f (step 114); and the remaining portion will not pass through the target voxel 14, and thus will not be frequency shifted by the pulse of ultrasound 32, resulting in a pulse of scattered background light 46 having a frequency f−fᵤₛ (the same frequency as the frequency shifted sample light 40 prior to entering the anatomical structure 16) that combines with the pulse of signal light 44 to create a pulse of a sample light pattern 47 (step 116).

Next, the interferometer 22 then combines (e.g., via the light combiner 58) the pulse of reference light 42 with the pulse of the sample light pattern 47 to generate a pulse of an interference light pattern 48 (initially, the interference light pattern pulse Iₐ illustrated in FIG. 5) (step 118). Then, under control of the controller 24, all of the detectors 68 (FIG. 4) of the lock-in camera 28 simultaneously detect respective spatial components of the interference light pattern 48 (i.e., speckle grains in the case where the interference light pattern includes a speckle light pattern) (step 120), and values (e.g., power values) representative of the spatial components of the interference light pattern 48 are stored in data storing bins 70 (initially, the first bins 70a of the corresponding detectors 68) (step 122).

At this point, only one quadrature measurement has been taken. If the interferometer 22 has not been set to all four of the phases (step 124), the controller 24 then repeats steps 102-122 to take the next quadrature measurement. That is, the next pulse of ultrasound 32 (e.g., ultrasound pulse U_{b} illustrated in FIG. 5) is generated and emitted into the anatomical structure 16 (step 102); the phase difference between the sample light 40 and the reference light 42 is set to the next pre-defined value (e.g., π/2) (step 104); the next pulse of source light 38 is generated (step 106) and split into the next pulse of sample light 40 (e.g., sample light pulse L_{b} illustrated in FIG. 5) and a pulse of reference light 42 (step 108); the next pulse of sample light 40 is frequency shifted by the ultrasound frequency fᵤₛ (step 110); the frequency shifted pulse of sample light 40 is delivered and diffusively scattered within the anatomical structure 16 (step 112); a portion of the scattered pulse of sample light 40 passing through the target voxel 14 is frequency shifted (tagged) by the pulse of ultrasound 32 passing through the target voxel 14, thereby generating the next pulse of scattered signal light 44 (step 114); the remaining portion of the scattered pulse of sample light 40 not passing through the target voxel 14 is not frequency shifted (not tagged) by the pulse of ultrasound 32 passing through the target voxel 14, thereby generating the next pulse of scattered background light 46 (step 116); the next pulses of reference light 42 and sample light pattern 47 are combined into the pulse of the next interference light pattern 48 (e.g., the interference light pattern pulse I_{b} illustrated in FIG. 5) (step 118); the spatial components of the next pulse of the interference light pattern 48 are detected (step 120); and the resulting spatial component values are stored in the data storing bins 70 (e.g., the second bins 70b of the corresponding detectors 68) (step 122).

Thus, it can be appreciated that steps 102-122 will be repeated to take the remaining quadrature measurements to generate and detect the pulses of the remaining interference light patterns (e.g., the third and fourth interference light pattern pulses I_{c}, I_{d} illustrated in FIG. 5) for the remaining phase settings (e.g., π and 3π/2) and ultimate storage of the spatial component values in the data storing bins 70 (e.g., the third and fourth bins 70c, 70d of the corresponding detectors 68).

After all four quadrature measurements have been taken, the controller 24 recalls the spatial component values of the detected interference light pattern pulses 48 from the data storing bins 70 of the lock-in camera 28 and transfers these values to the processor 30 (step 126). The processor 30 reconstructs the amplitude of the signal light 44 from the four interference light patterns 48 based on these spatial component values (e.g., by using the quadrature equation [2]) (step 128). Steps 102-128 can be iterated to repeatedly acquire data measurements of the target voxel 14, and if a sufficient number of data measurements have been acquired (step 130), the processor 30 may then determine the physiologically-dependent optical parameter (e.g., level of deoxygenated and/or oxygenated hemoglobin concentration or relative abundance or level of water concentration or relative water concentration) of the target voxel 14 based on the data measurements (step 132). In the case where the target voxel 14 is brain matter, the processor 30 may further determine the level of neural activity within the target voxel 14 based on the determined physiologically-dependent optical parameter (step 134).

For example, if the physiologically-dependent optical parameter is the level of deoxygenated and/or oxygenated hemoglobin concentration or relative abundance, and if the amplitude of the signal light 44 is relatively low (or high), indicating high absorption of light by blood in the target voxel 14, it can be assumed that there is a relatively high (or low) hemodynamic response (depending on the light wavelength used) through the target voxel 14, and thus, a substantial amount of neural activity in the target voxel 14. In contrast, if the amplitude of the signal light 44 is relatively high (or low), indicating low absorption of light by blood in the target voxel 14, it can be assumed that there is a relatively low hemodynamic response (depending on the wavelength) through the target voxel 14, and thus, comparatively little neural activity in the target voxel 14.

If the physiologically-dependent optical parameter is the level of water concentration or relative water concentration, and if the amplitude of the signal light 44 greatly varies over a short period of time, indicating a fast signal of neural activity in the brain tissue, it can be assumed that there is a substantial amount of neural activity in the target voxel 14. In contrast, if the amplitude of the signal light 44 varies very little over a short period of time, indicating that there is no fast signal of neural activity in the brain matter, it can be assumed that there is very little or no neural activity in the target voxel 14.

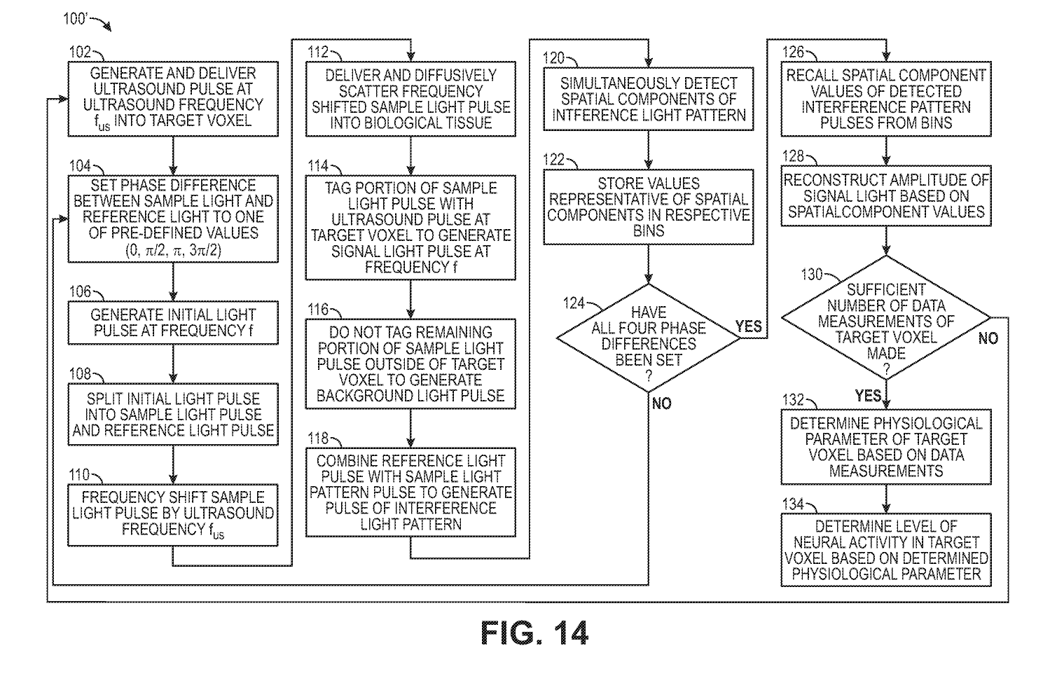

Referring to FIG. 14, another particular method 100′ performed by the UOT system 10 to non-invasively measure the target voxel 14 in the anatomical structure 16 will now be described. This particular method 100′ implements the pulsing sequence of FIG. 7.

The method 100′ is similar to the method 100 illustrated in FIG. 13, with the exception that, instead of a one-to-one correspondence between the sample light pulse and the ultrasound pulse, multiple sample pulses (in this case, four) are delivered to the biological tissue for every ultrasound pulse delivered to the biological tissue. Thus, after a quadrature measurement, the process returns to step 104 (instead of step 102) to take the next quadrature measurement.

That is, during the delivery of current pulse of ultrasound 32 (e.g., ultrasound pulse U illustrated in FIG. 5), the phase difference between the sample light 40 and the reference light 42 is set to the next pre-defined value (e.g., π/2) (step 104); the next pulse of source light 38 is generated (step 106) and split into the next pulse of sample light 40 (e.g., sample light pulse L_{b} illustrated in FIG. 5) and a pulse of reference light 42 (step 108); the next pulse of sample light 40 is frequency shifted by the ultrasound frequency fᵤₛ (step 110); the frequency shifted pulse of sample light 40 is delivered and diffusively scattered within the anatomical structure 16 (step 112); a portion of the scattered pulse of sample light 40 passing through the target voxel 14 is frequency shifted (tagged) by the pulse of ultrasound 32 passing through the target voxel 14, thereby generating the next pulse of scattered signal light 44 (step 114); the remaining portion of the scattered pulse of sample light 40 not passing through the target voxel 14 is not frequency shifted (not tagged) by the pulse of ultrasound 32 passing through the target voxel 14, thereby generating the next pulse of scattered background light 46 that combines with the pulse of signal light 44 to create a pulse of the sample light pattern 47 (step 116); the next pulses of reference light 42 and sample light pattern 47 are combined into the pulse of the next interference light pattern 48 (e.g., the interference light pattern pulse I_{b} illustrated in FIG. 5) (step 118); the spatial components of the next pulse of the interference light pattern 48 are detected (step 120); and the resulting spatial component values are stored in the data storing bins 70 (e.g., the second bins 70 of the corresponding detectors 68) (step 122).

After all four quadrature measurements have been taken at steps 102-124, as in the manner described above with respect to the method 100 of FIG. 13, the spatial component values of the detected interference light pattern pulses 48 are recalled from the data storing bins 70 of the lock-in camera 28 (step 126); and the amplitude of the signal light 44 is reconstructed from the four interference light patterns 48 based on these spatial component values (step 128). Steps 102-128 can be iterated to repeatedly acquire data measurements of the target voxel 14, and if a sufficient number of data measurements have been acquired (step 130), the physiologically-dependent optical parameter (e.g., the level of deoxygenated and/or oxygenated hemoglobin concentration or relative abundance or level of water concentration or relative water concentration) of the target voxel 14 is determined based on the data measurements (step 132), and the level of neural activity within the target voxel 14 is determined based on the determined physiologically-dependent optical parameter (step 134).

Although methods 100 and 100′ have been described in the context of a four-bin quadrature scheme, methods 100 and 100′ may alternatively be operated in a two-bin scheme. In this case, instead of setting one of four different phase differences in step 104, only two phase differences are set (0 and π) to generate two phase-modulated interference patterns, and the processor 30 reconstructs the amplitude of the signal light 44 from the two interference light patterns 48 based on these spatial component values (e.g., by using the equations [6]-[10]).

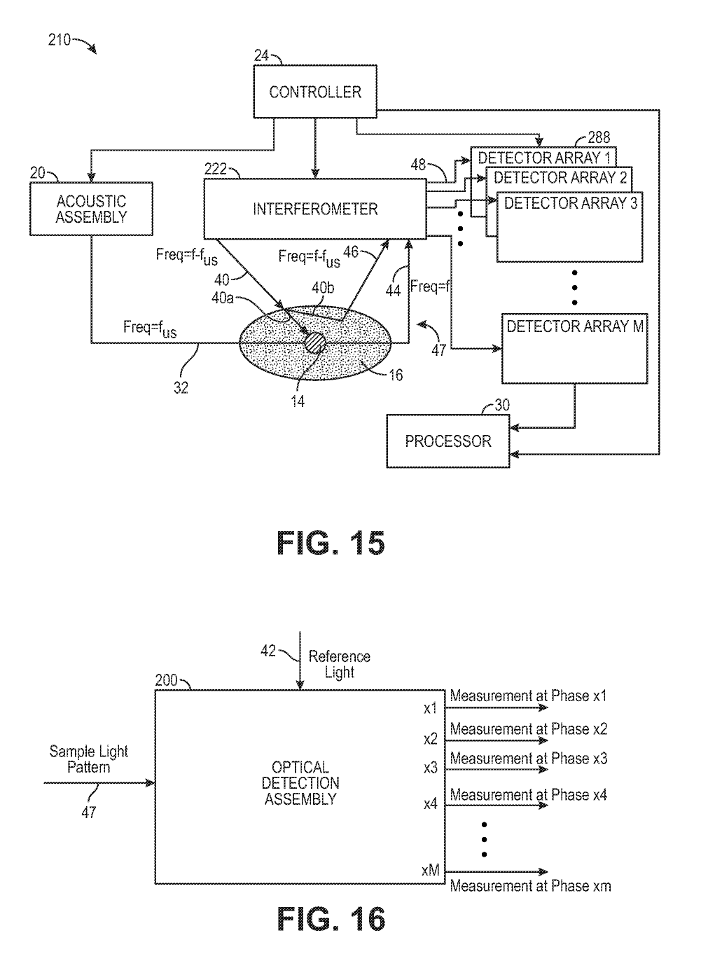

Referring now to FIG. 15, a UOT system 210 constructed in accordance with another embodiment of the present inventions will be described. The UOT system 210 is similar to the UOT system 10 illustrated in FIG. 1 in that it is designed to non-invasively measure a physiologically-dependent optical parameter (which may be the same types of parameters discussed above with respect to the UOT system 10) of a target voxel 14 in an anatomical structure 16. As with the UOT system 10, in a practical implementation, the UOT system 210 will acquire data from multiple target voxels 14 spatially separated from each other within a volume of interest (not shown). For purposes of brevity, the UOT system 210 will be described as acquiring one data voxel (i.e., data representative of a physiologically-dependent optical parameter of the target voxel 14), although it should be understood that the UOT system 210 may be capable of acquiring more than one data voxel from the volume of interest of the anatomical structure 16. The UOT system 210 differs from the UOT system 10 in that, instead of sequentially performing an M number of measurements of the target voxel 14, the UOT system 210 concurrently performs an M number of measurements of the target voxel 14.

To this end, the UOT system 210 comprises an interferometer 222 that, unlike the interferometer 22 of the UOT system 10 (which sequentially generates an M number of interference patterns 48 via phase modulation), concurrently generates an M number of interference patterns 48 via phase modulation. The UOT system 210 further comprises an M number of optical detector (or pixel) arrays 228 (e.g., using multiple cameras or dedicated spatial regions of a single camera) that, unlike the lock-in camera 28 of the UOT system 10 (which detects the interference light patterns 48 using a single array of detectors in the form of a lock-in camera 28 (in each of several successive data storing bins associated with each detector (or pixel)), are optically registered with each other to concurrently detect the interference light patterns 48 over the M number of phases.

Referring to FIG. 16, the optical detection portion of the interferometer 222 that concurrently generates the M number of interference patterns 48, as well as the M number of optical detector arrays 228, may be topologically combined into an optical detection assembly 200, various specific embodiments of which will be described in further detail below. Such optical detection assembly 200 (including the specific embodiments thereof described below) is configured for receiving the sample light pattern 47 and reference light 42, and outputting an M number of phase shifted measurements. In some embodiments described, the optical detection assembly 200 may optionally comprise on-board processing that computes and outputs one or more values (representative of the desired physiologically-dependent optical parameter) from the M number of measurements.

Although the specific embodiments of the optical detection assembly 200 described below utilize a two-measurement detection scheme (M=2) or a four-measurement detection scheme (M=4), the optical detection assembly 200 illustrated in FIG. 16 can be generalized to provide any combinations of phase differences between the outputted measurements by simply introducing the appropriate phase lags to the measurements. Thus, the optical detection assembly 200 can be viewed as a “black box” that receives the sample light pattern 47 and reference light 40 as inputs, and outputting an M number of measurements over different phases. While lock-in cameras may be scaled to have more data storing bins (e.g., twice as many), doing so implies changes to the electronic/very large scale integration (VLSI) design of lock-in camera pixels, which may detrimentally impact fill factor, speed of operation, bin saturation level, number of pixels, custom silicon chip design and fabrication, and so forth, specific embodiments of the generalized optical detection assembly 200 can be designed with arbitrarily large numbers of output ports (M is arbitrary) and high pixel counts, simply by introducing, e.g., more beam splitters, reference shifts, and camera alignments, as will be shown in further detail below.

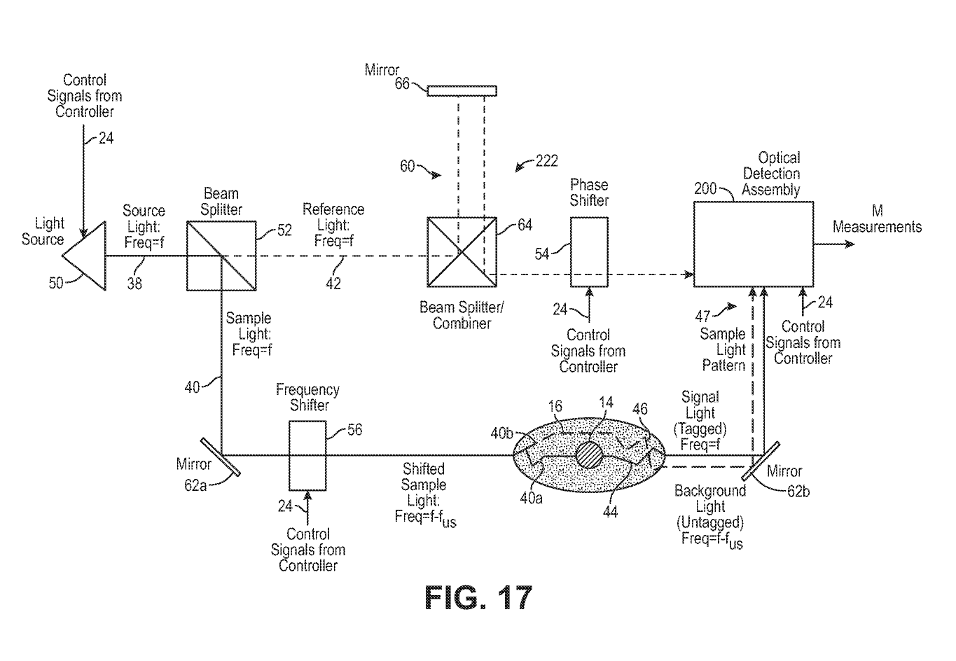

Referring to FIG. 17, it can be seen that the interferometer 222 of the UOT system 210 is identical to the interferometer 22 of the UOT system 10 illustrated in FIG. 3a, with the exception that the detection portion of the interferometer, along with cameras, are subsumed into an optical detection assembly 200. Of course, the interferometer 222 of the UOT system 210 can alternatively be identical to the interferometers 22 of the UOT system illustrated in FIGS. 3b-3d. In essence, the light combiner 58, as well as the lock-in camera 28, are replaced with the optical detection assembly 200 illustrated in FIG. 16. Although optical detection assemblies 200 are described herein in the context of a UOT system 210 that combines the signal light 44 and reference light 42 in a homodyne manner, it should be appreciated that optical detection assemblies 200 described herein may be alternatively used in the context of other systems that combine the signal light 44 and reference light 42 in a heterodyne manner.

Although the UOT system 210 is described herein as non-invasively measuring one target voxel 14 at a time, the UOT system 10 may be modified to image multiple target voxels 14 at a time. For example, in one particular embodiment, an acoustic assembly may simultaneously generate multiple foci in parallel (i.e., focuses the ultrasound on multiple target voxels at the same time), with each ultrasound focus being modulated at a different optical frequency (e.g., f₁−fᵤₛ, f₂−fᵤₛ, f₃−fᵤₛ, etc.), with separate frequency-shifted reference light (e.g., f₁, f₂, f₃, etc.) being used to interfere with the resulting signal light in a homodyne manner. This enables parallelization of the UOT detection. Separate optical detection assemblies 200, each having an M number of detector arrays, may be utilized to detect the different interference patterns corresponding to the different target voxels.

By using the optical detection assembly 200 in applications such as in UOT, lock-in detection can be made essentially instantaneous, limited only by the achievable pulse durations of the sample light (e.g., in the context of UOT, a 1 microsecond optical pulse width when used in conjunction with a 1 MHz ultrasound frequency, or a 0.5 microsecond optical pulse width used when used in conjunction with a 2 MHz ultrasound frequency), which would correspond, for instance via the operative speckle decorrelation times, to imaging depths into tissue of many centimeters.

Thus, the following embodiments of optical detection assemblies 200 can overcome the problem of short speckle de-correlation times for imaging at great depths inside dynamic, strongly scattering biological tissues, enabling UOT and other related methods to address the problem of in vivo deep-tissue imaging, e.g., through many millimeters of human skull followed by many millimeters of brain tissue. Not only can these optical detection assemblies 200 be used to detect blood-oxygen-level dependent signals, flow response, or other physiological dependent optical parameters indicative of localized changes in neural activity in the human brain, it is contemplated that these optical detection assemblies 200 can be used to detect changes faster than hemodynamic responses through the human skull. These optical detection assemblies 200 can also be used with non-endogenous contrast agents, such as protein-based calcium or voltage indicators that optically report neuronal activity (see Broussard G J, Liang R, Tian L, “Monitoring Activity in Neural Circuits with Genetically Encoded Indicators,” Frontiers in Molecular Neuroscience, Vol. 7 (2014).

Furthermore, the following embodiments of detection assemblies 200 can be used with conventional cameras with high pixel counts, effectively turning any conventional camera into a lock-in camera, and thus, dramatically expanding the achievable scale of lock-in detection of optical wavefronts. For application to UOT, this means that many more optical speckle grains can be sampled, thereby increasing signal-to-noise ratio. For example, conventional cameras with 10 or 100 or more megapixels can be utilized for lock-in detection in the following embodiments of the UOT system, potentially increasing the scale of lock-in detection of optical wavefronts by a factor of approximately 1000 over the current state of art (e.g., as represented by the Heliotis HeliCam C3 lock-in camera with less than 100,000 pixels).

Thus, the following embodiments of optical detection assemblies 200 are sufficiently sensitive to be useful for through-human-skull functional neuroimaging by enabling high-pixel count based lock-in detection and demodulation of the scattered optical wavefront emanating from a subject's head, and doing so even in the presence of the fast speckle decorrelations characteristic of deep tissue imaging. For example, a UOT system 210 that uses a 1-megapixel conventional camera with a 7 KHz frame rate, 1 ultrasound pulse per frame, and a 1 microsecond optical pulse duration, may have an imaging depth of approximately 10 centimeters in living brain tissueenough for whole brain coverage in a human. This suggests that the UOT system 210 could access the entire human brain, given sufficient detector sensitivity, for which the achievable signal-to-noise ratio scales with the square root of the number of camera pixels used and with the square root of the number of temporal averages taken per voxel.

Although the optical detection assemblies 200 are described and illustrated herein in the context of the UOT system 210, the optical detection assemblies 200 described herein can be used in any optical detection system (including pulsed or continuous UOT) where it is desirable to takes multiple measurements of a target voxel 14 simultaneously or in quick succession. For example, in the context of quasi-ballistic/snake photon Optical Coherent Tomography (“snake photon OCT”), which path-length encodes the signal light scattered by the target voxel 14, in contrast to UOT, which frequency encodes the signal light scattered by the target voxel 14, the optical detection assemblies 200 described herein can be used to simultaneously detect interference light patterns output by the interferometers of snake photon OCT systems.

For example, instead of frequency encoding the signal light 44 with ultrasound 32, snake photon OCT systems may average the detected intensity values of interference patterns on a pixel-by-pixel basis to compute an estimate of the magnitude of the signal light within a user-defined path-length range that approximates straight travel paths in tissue (i.e., quasi-ballistic photons that have experienced some degree of scattering but have a path length that is within a light source coherence length from the path length of the reference optical path), as well as the detection of photons that have a straight travel path in tissue (i.e., ballistic photons that have a path length that matches the path length of the reference optical path). Further detail discussing snake photon OCT systems are set forth in U.S. Provisional Patent Application Ser. No. 62/599,510, entitled “Systems and Methods for Quasi-Ballistic Photon Optical Coherence Tomography in Diffusive Scattering Media Using a Lock-In Camera Detector,” filed Dec. 15, 2017, which is expressly incorporated herein by reference; and U.S. patent application Ser. No. 15/853,538, entitled “Systems And Methods for Quasi-Ballistic Photon Optical Coherence Tomography in Diffusive Scattering Media Using a Lock-In Camera Detector,” filed on Dec. 22, 2017, which is also expressly incorporated herein by reference.

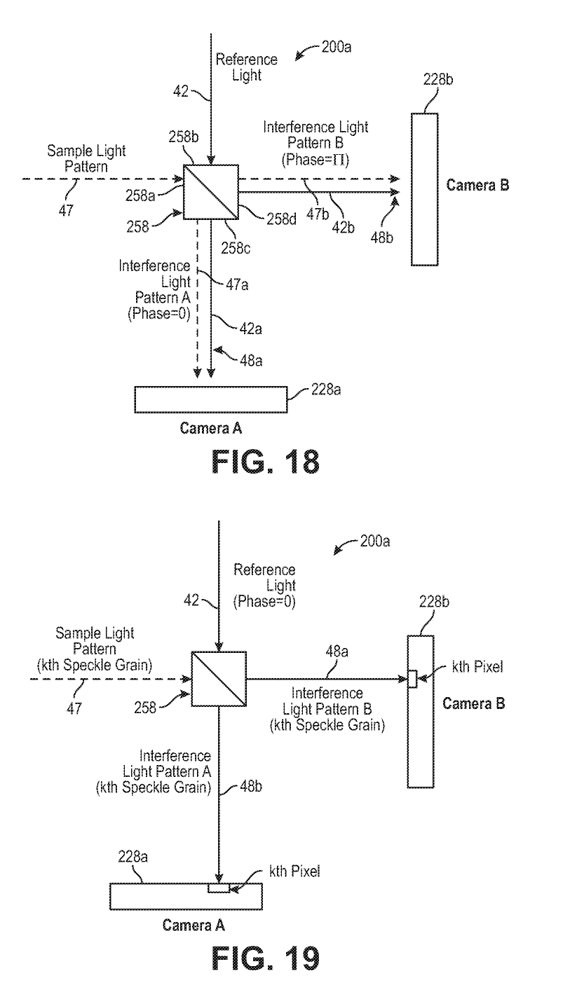

Referring to FIG. 18, one specific embodiment of an optical detection assembly 200a is capable of making two separate measurements of the target voxel 14 (shown in FIG. 17) simultaneously or in short succession by measuring the interference between the sample light field 47 and reference light 42 at two separate phases differing from each other by an angular phase of π.

To this end, the optical detection assembly 200a comprises a single beam splitter/combiner 258 (which replaces the light combiner 58 of the interferometer 22 illustrated in FIGS. 3a-3d). The beam splitter/combiner 258 is configured for splitting the reference light 42 respectively into reference light 42 having an M number of different phases relative to the sample light pattern 47 (and in this case, reference light 42a, 42b respectively having two different phases of 0 and π), splitting the sample light pattern 47 respectively into sample light pattern portions 47a, 47b, and concurrently combining the sample light pattern portions 47a, 47b and the reference light 42a, 42b to respectively generate an M number of interference light patterns 48 (in this case, two interference light patterns 48a (“Interference Light Pattern A”), 48b (“Interference Light Pattern B”)).

That is, the sample light pattern 47 enters an input port 258a of the beam splitter/combiner 258, where it is split into a reflected sample pattern portion 47a and a transmitted sample light pattern portion 47b, and the reference light 42 enters another input port 258b of the beam splitter/combiner 258, where it is split into a transmitted reference light 42a and a reflected reference light 42b. In a simultaneous manner, the reflected sample light pattern portion 47a interferes with the transmitted reference light 42a to generate the interference light pattern 48a, and the transmitted sample light pattern portion 47b interferes with the reflected reference light 42b to generate the interference light pattern 48b.

Due to power conservation, a four-port network, such as the beam splitter/combiner 258, requires the total power entering the input ports 258a, 258b to be equal to the total power exiting the output ports 258c, 258d, and thus, the transmitted reference light 42a will have a nominal phase of 0, and the reflected reference light 42b will have a phase of π. That is, as will be described in further detail below, since the combined power of the DC terms of the interference light patterns 48a, 48b exiting the respective output ports 258a, 258b of the beam splitter/combiner 258 will be equal to the combined power of combined DC power of the sample light pattern 47 and reference light 42 respectively entering the input ports 258a, 258b of the beam splitter/combiner 258, the interfering AC beat pattern terms of the respective interference light patterns 48a, 48b will need to differ in phase by 180 degrees such that they sum to zero.

Although a lock-in camera with two data storing “bins” can be used to detect the two interference light patterns 48a, 48b, in such a scenario, the interference light patterns 48a, 48b would have to be detected sequentially, with the resulting values stored in the two bins in rapid succession, preferably with the total time to acquire the two measurements being shorter than the speckle de-correlation time of the target voxel 14, which will fall into the microsecond range or below if the target voxel 14 is multiple centimeters deep within the anatomical structure 16 (shown in FIG. 17).

However, instead of using a conventional lock-in camera, the optical detection assembly 200a comprises an M number of arrays of detectors (or pixels) 228 (in this case, two arrays of detectors in the form of cameras 228a (“Camera A”), 228b (“Camera B”) respectively disposed at two output ports 258c, 258d of the beam splitter/combiner 258 for concurrently detecting the respective two interference light patterns 48a, 48b, and generating M pluralities of values representative of intensities of the spatial components (“speckle grains”) of the respective M number of interference light patterns (in this case, two pluralities of values representative of spatial components of the interference light patterns 48a, 48b). Thus, the sample light field 47 and reference light 42 combine to project an interference light pattern 48a onto the camera 228a, and likewise to project an interference light pattern 48b onto the camera 228b, but with respect to a different phase of the reference light 42. In the illustrated embodiment, the planes of the cameras 228a, 228b are perpendicular to each other, such that they face the respective output ports 258c, 258d of the beam splitter/combiner 258. The cameras 228a, 228b may be conventional in nature (e.g., readily available conventional charge-coupled device (CCD) cameras).

Although the cameras 228a, 228b are separate and distinct, the cameras 228a, 228b are optically aligned with each other, such that any given pixels on the cameras 228a, 228b have a known one-to-one correspondence with each other. That is, as illustrated in FIG. 19, a spatial component of the sample light pattern 47 (i.e., the kth speckle grain of the speckle light field) interferes with the reference light 42a with no phase shift (i.e., 0) (shown in FIG. 18) to generate a kth speckle grain of the interference light pattern 48a that is detected by kth pixel of the camera 228a, and the same kth speckle grain of the sample light pattern 47 interferes with the reference light 42b with a phase shift (i.e., π) (shown in FIG. 18) to generate a corresponding kth speckle grain of the interference light pattern 48b that is detected by the corresponding kth pixel of the camera 228b. Since the kth pixel of the camera 228a has a known correspondence via optical alignment with the kth pixel of the camera 228b, the pair of intensity values detected by the kth pixels of the cameras 228a, 228b serve as the analog to the pair of intensity values that would normally be stored by the kth pixel in the two data storing bins of a lock-in camera. It should be appreciated that although FIG. 19 illustrates one speckle grain “k,” an equivalent process for measuring the speckle grain k takes place for all speckles grains in parallel in the manner of imaging an entire speckle light field.

At each corresponding pair of kth pixels, the optical power received by the respective cameras 228a, 228b is equal to the summation of the power of the reference light 42 (P_{reference}A and P_{reference}B) input into the beam splitter/combiner 258, the sample light pattern 47 (PₛₐₘₚₗₑA and PₛₐₘₚₗₑB) input into the beam splitter/combiner 258, and an interference term between the reference light 42 and sample light pattern 47 (P_{interfere}A and P_{interfere}B). By the power conservation, the interference terms P_{interfere}A and P_{interfere}B are 180 degrees out of phase for the cameras 228a, 228b.

It follows that, in the context of pulsed UOT, the power detected at a kth pixel for the first camera 228a can be expressed as:

Value_{1,k}=P_{background}+P_{signal}+P_{reference}+2(P_{signal}×P_{reference})^{1/2}×cos(φ_{unknown1,speckle k})+2(P_{signal}×P_{background})^{1/2}×cos(2π×fᵤₛ−φ_{unknown2,speckle k})+2(P_{reference}×P_{background})^{1/2}×cos(2π×fᵤₛ−φ_{unknown3,speckle k}), [15]

and likewise, the power detected at the corresponding kth pixel of the second camera 228b can be expressed as:

Value_{2,k}=P_{background}+P_{signal}+P_{reference}+2(P_{signal}×P_{reference})^{1/2}×cos(φ_{unknown1,speckle k}+π)+2(P_{signal}×P_{background})^{1/2}×cos(2π×fᵤₛ−φ_{unknown2,speckle k}+π)+2(P_{reference}×P_{background})^{1/2}×cos(2π×fᵤₛ−φ_{unknown3,speckle k}+π); [16]

where P_{background} represents light at frequency f+fᵤₛ that has not been tagged with the ultrasound 32; P_{signal} represents light at frequency f that has been tagged with the ultrasound 32; P_{reference} represents the reference light at frequency f; and φ_{unknown1, speckle k}, φ_{unknown2, speckle k}, and φ_{unknown3, speckle k} are random phases at the kth speckle grain at the time of measurement, which originate via multiple scattering of coherent light inside the tissue.

The terms 2(P_{signal}×P_{background})^{1/2}×cos(2π×fᵤₛ−φ_{unknown2})+2(P_{reference}×P_{background})^{1/2}×cos(2π×fᵤₛ−φ_{unknown3}) oscillate at the frequency fᵤₛ, and are not detected by the cameras 228a, 228b given appropriate light pulse duration and integration time, and thus, can be ignored. As such, equations [15] and [16] can be respectively reduced to:

Value_{1,k}=P_{background}+P_{signal}+P_{reference}+2(P_{signal}×P_{reference})^{1/2}×cos(φ_{unknown}); and [17]

Value_{2,k}=P_{background}+P_{signal}+P_{reference}+2(P_{signal}×P_{reference})^{1/2}×cos(φ_{known}+π [18]

It is noted that the term P_{background}+P_{signal}+P_{reference} in each of equations [17] and [18] represents the DC terms of the interference light patterns 48a, 48b, whereas the terms 2(P_{signal}×P_{reference})^{1/2}×cos(φ_{unknown}) and 2(P_{signal}×P_{reference})^{1/2}×cos(φ_{unknown}+π) in the respective equations [17] and [18] represent the AC beat patterns of the interference light patterns 48a, 48b.

The terms P_{background}+P_{signal}+P_{reference} are constant across the two cameras 228a, 228b, and thus, can be eliminated by taking the difference of the intensity values Value_{1,k} and Value_{2,k}, leaving only the difference of the interference terms, as 2(P_{signal}×P_{reference})^{1/2}× cos(φ_{unknown})−2(P_{signal}×P_{reference})^{1/2}×cos(φ_{unknown}+π), which can be summed over all of the k pixels and then averaged in accordance with the following equation:

2(P_{signal}×P_{reference})^{1/2}=(Σk|Value_{1,k}−Value_{2,k}|)/N [19]

across all k=1,2, . . . N speckles or pixels,
i.e., the average across all pixels of the absolute value of the difference across corresponding pixels. Based on known scaling relationship between the power of the reference light P_{reference} and the power of the signal light P_{signal} discussed above, the power of the P_{signal} can be expressed as:

P_{signal}∝(Σk|Value_{1,k}−Value_{2,k}|)/N, [20]

i.e., the average across all pixels of the absolute value of the difference across corresponding pixels.

It should be noted that other sign-rectifying operations of the intensity value differences besides taking the absolute can be utilized to obtain the power of the signal light 44. For example, a squaring operation can be applied to the intensity value differences. Thus, the power of the signal light 44 can alternatively be computed as the square of the difference between the two intensity values stored in the two data storing bins 70 for each pixel 68 and then averaged in accordance with the following equation:

P_{signal}∝((Value_{1,k}−Value_{2,k})²)/N [21]

across all k=1,2, . . . N speckles or pixels.

Thus, it can be appreciated that the intention of the two-bin measurement is to arrive at a precise estimate of power of the signal light P_{signal} up to the aforementioned scaling relationship defined by the strength of the reference light P_{reference}, by removing the terms that are constant between the two measurements of the cameras 228a, 228b, removing the unknown speckle-specific phases, and extracting only the amplitude of the cosine term. As with the two-bin detection scheme described above with respect to the UOT system 10, with an N number of pixels, the signal-to-noise ratio (SNR) scales with N^{1/2} in accordance with equation [13] set forth above.

In the context of the UOT, just as with the detection schemes described above, this detection scheme serves as a measurement of a physiologically-dependent optical parameter at the target voxel 14.

It should be noted that if the split ratio of the beam splitter/combiner 258 is not 50/50, the interference terms would still be 180 degrees out of phase, i.e., P_{interfere}A=−P_{interfere}B by the power conservation. In the case where the power of the reference light 42 is ramped up to achieve shot noise limited detection, then P_{reference}A and P_{reference}B are the dominant constant terms, which would no longer be equal across the cameras 228a, 228b, i.e., P_{reference}A will not be equal to P_{reference}B. The subtraction process represented by equations [20] or [21] can then be modified to use a scaled subtraction to zero out the constant offset during subtraction. This scaling ratio can be determined a priori. The resulting difference will still be a quantifiable measure of the interference term, and hence, in the context of UOT, of the UOT tagged photon signal strength.

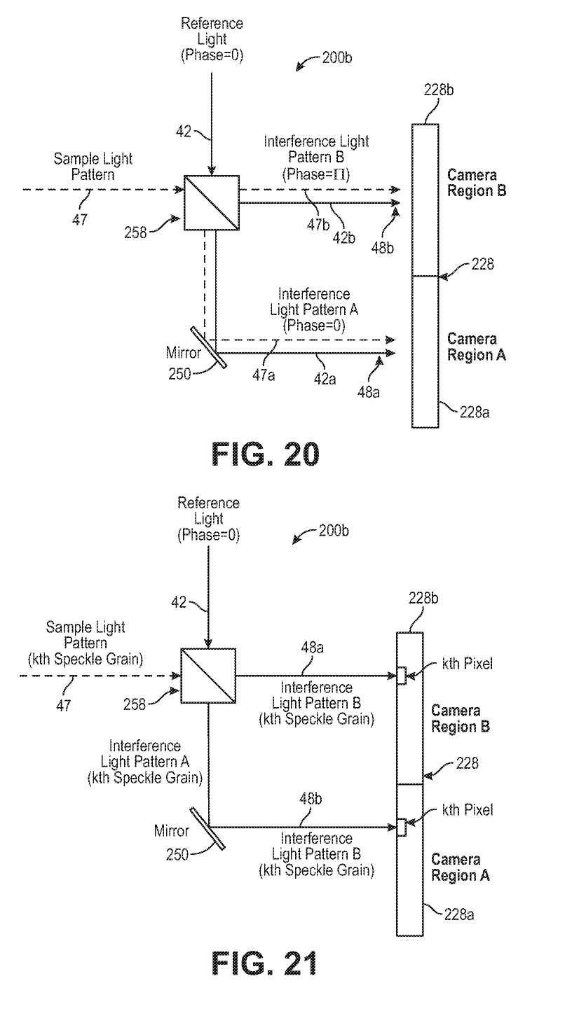

Although the optical detection assembly 200a has been described and illustrated as comprising detector arrays that take the form of separate cameras 228a, 228b (in effect, the detector arrays are mechanically disposed on separate camera microchips), an optical detection assembly 200b can comprise detection arrays that take the form of a single camera 228 (in effect, the detector arrays are mechanically disposed on a single camera microchip), as illustrated in FIG. 20.

As with the optical detection assembly 200a, the beam splitter/combiner 258 of the optical detection assembly 200b is configured for splitting the reference light 42 respectively into reference light 42 having an M number of different phases relative to the sample light pattern 47 (and in this case, reference light 42a, 42b respectively having two different phases of 0 and π), splitting the sample light pattern 47 respectively into sample light pattern portions 47a, 47b, and concurrently combining the sample light pattern portions 47a, 47b and the reference light 42a, 42b to respectively generate an M number of interference light patterns 48 (in this case, two interference light patterns 48a (“Interference Light Pattern A”), 48b (“Interference Light Pattern B”)).

However, instead of respectively projecting the interference light patterns 48a, 48b onto two cameras, the beam splitter/combiner 258 of the optical detection assembly 200b projects the interference light patterns 48a, 48b from its respective output ports onto two separate spatial regions 248a, 248b of the same camera 228. To this end, the optical detection assembly 200b comprises an angled mirror 250 configured for directing the interference light pattern 48a from the beam splitter/combiner 258 towards the same plane to which the interference light pattern 48b is directed, i.e., towards the single camera 228.

In the same manner as the cameras 228a, 228b are optically aligned with each other in the optical detection assembly 200a, the spatial regions 228a, 228b of the single camera 228 of the optical detection assembly 200b are optically aligned with each other, such that any given pixels on the spatial regions 228a, 228b have a known one-to-one correspondence with each other. That is, as illustrated in FIG. 21, the kth speckle grain of the sample light pattern 47 interferes with the reference light 42a with no phase shift (i.e., 0 phase) to generate a kth speckle grain of the interference light pattern 48a that is detected by the kth pixel of the spatial region 228a of the camera 228, and the same kth speckle grain of the sample light pattern 47 interferes with the reference light 42b with a phase shift (i.e., π) to generate a corresponding kth speckle grain of the interference light pattern 48b that is detected by the kth pixel of the spatial region 228b of the camera 228.

An estimate of the power of the signal light P_{signal} can be determined in accordance with equations [20] or [21], where Value_{1,k} is the power detected at a kth pixel for the first region 228a of the camera 228, and Value_{2,k} is the power detected at a kth pixel for the second region 228b of the camera 228.

Although the optical detection assembly 200a has been described and illustrated as being capable of making two separate measurements of the target voxel 14 (shown in FIG. 17) simultaneously or in short succession by measuring the interference between the sample light field 47 and reference light 42 at two separate phases of the reference light 42, an optical detection assembly 200 may be capable of making more than two separate measurements of the target voxel 14 (shown in FIG. 17) simultaneously or in short succession by measuring the interference between the sample light field 47 and reference light 42 at more than two separate phases of the reference light 42.

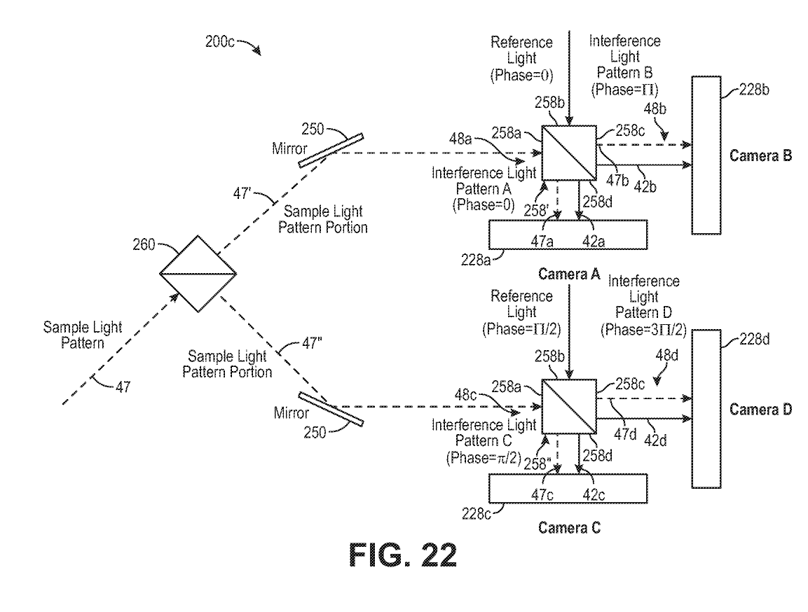

For example, one embodiment of an optical detection assembly 200c is capable of making four separate measurements of the target voxel 14 simultaneously or in short succession by measuring the interference between the sample light field 47 and reference light 42 at four separate phases (e.g., 0, π/2, π, and 3π/2) of the reference light 42, as illustrated in FIG. 22.

To this end, the optical detection assembly 200c comprises at least one beam splitter 260 configured for splitting the sample light pattern 47 into a plurality of sample light pattern portions, and in this case, a single beam splitter 260 that splits the sample light pattern 47 into first and second sample light pattern portions 47′, 47″.

The optical detection assembly 200c further comprises a first beam splitter/combiner 258′ configured for splitting the reference light 42 (having a nominal phase of 0) respectively into a first two of the four different phases relative to the sample light pattern 47 (and in this case, reference light 42a, 42b respectively having two different phases of 0 and π), splitting the first sample light pattern portion 47′ respectively into sample light pattern portions 42a, 42b, and concurrently combining the sample light pattern portions 47a, 47b and reference light 42a, 42b to respectively generate the first two of the four interference light patterns 48a (“Interference Light Pattern A”), 48b (“Interference Light Pattern B”).

That is, the first sample light pattern portion 47′ enters an input port 258a of the first beam splitter/combiner 258′, where it is split into a reflected sample pattern portion 47a and a transmitted sample light pattern portion 47b, and the reference light 42 (having a phase of 0) enters another input port 258b of the first beam splitter/combiner 258′, where it is split into a transmitted reference light 42a having a phase of 0 and a reflected reference light 42b having a shifted phase of π. In a simultaneous manner, the reflected sample light pattern portion 47a interferes with the transmitted reference light 42a to generate the interference light pattern 48a, and the transmitted sample light pattern portion 47b interferes with the reflected reference light 42b to generate the interference light pattern 48a.

The optical detection assembly 200c further comprises a second beam splitter/combiner 258″ configured for splitting the reference light 42 (having a nominal phase of π/2) respectively into a second two of the four different phases relative to the sample light pattern 47 (and in this case, reference light 42c, 42d respectively having two different phases of π/2 and 3π/2), splitting the second sample light pattern portion 47″ respectively into sample light pattern portions 42c, 42d, and concurrently combining the sample light pattern portions 47c, 47d and the reference light 42c, 42d to respectively generate the second two of the four interference light patterns 48c (“Interference Light Pattern C”), 48d (“Interference Light Pattern D”).

That is, the second sample light pattern portion 47″ enters an input port 258a of the second beam splitter/combiner 258″, where it is split into a reflected sample pattern portion 47c and a transmitted sample light pattern portion 47d, and the reference light 42 (having a phase of π/2) enters another input port 258b of the second beam splitter/combiner 258″, where it is split into a transmitted reference light 42c having a phase of π/2 and a reflected reference light 42d having a shifted phase of 3π/2. In a simultaneous manner, the reflected sample light pattern portion 47c interferes with the transmitted reference light 42c to generate the interference light pattern 48c, and the transmitted sample light pattern portion 47d interferes with the reflected reference light 42d to generate the interference light pattern 48d.

The optical detection assembly 200c further comprises a pair of angled mirrors 250 configured for respectively redirected the sample light pattern portion 47′, 47″ to the respective first and second beam splitter/combiners 258′, 258″.

Notably, as discussed above, the reference light 42 that enters the first beam splitter/combiner 258′ has a nominal phase shift of 0, and the reference light 42 that enters the second beam splitter/combiner 258″ has a nominal phase shift of π/2 (in order to get the full quadrature of phases), which can be accomplished by splitting the reference light 42 into two lines, and phase shifting one of the lines relative to the other line by π/2. The optical detection assembly 200c comprises a pair of angled mirrors 250a, 250b for respectively directing the first and second sample light patterns 47a, 47b towards the input ports 258a of the first and second beam splitter/combiners 258′, 258″.

Although a lock-in camera with four data storing “bins” can be used to detect the four interference light patterns 48a-48d, in such a scenario, the interference light patterns 48a-48d would have to be detected sequentially, with the resulting values stored in the four bins in rapid succession, preferably with the total time to acquire the four measurements being shorter than the speckle de-correlation time of the target voxel 14, which, as briefly discussed above, will fall below a microsecond if the target voxel 14 is multiple centimeters deep within the anatomical structure 16 (shown in FIG. 16).

However, instead of using a conventional lock-in camera, the optical detection assembly 200c comprises four arrays of detectors (or pixels) 228 (in this case, four arrays of detectors in the form of cameras 228a (“Camera A”), 228b (“Camera B”), 228c (“Camera C”), and 228d (“Camera D”), respectively disposed at the output ports 258c, 258d of the first and second beam splitter/combiners 258′, 258″ for concurrently detecting the respective four interference light patterns 48a-48d, and generating four pluralities of values representative of intensities of the spatial components of the respective four interference light patterns 48a-48d. Thus, the sample light field 47 and reference light 42 combine to project an interference light pattern 48a onto the camera 228a, project an interference light pattern 48b onto the camera 228b, project an interference light pattern 48c onto the camera 228c, and project an interference light pattern 48d onto the camera 228d, all with respect to different phases of the reference light 42. In the illustrated embodiment, the planes of the cameras 228a, 228b are perpendicular to each other, such that they face the respective output ports 258c, 258d of the first beam splitter/combiner 258′, and the planes of the cameras 228c, 228d are perpendicular to each other, such that they face the respective output ports 258c, 258d of the second beam splitter/combiner 258″.

It should be appreciated that as the number of measurements increases (e.g., from two to four) using a lock-in camera, the total time to acquire the measurements increases in a commensurate manner, whereas as the number of measurements increases (e.g., from two to four) using the optical detection assemblies 200 described herein, the total time to acquire the measurements remains essentially the same.

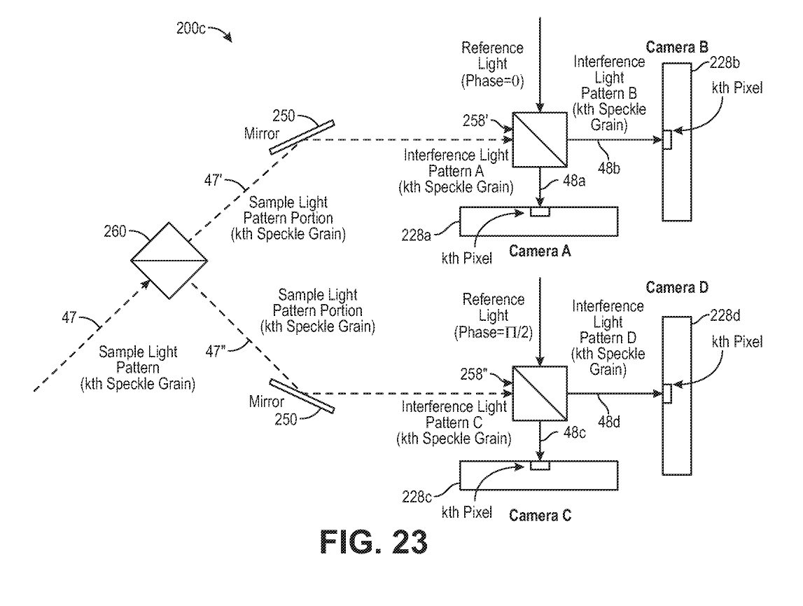

In the same manner as the cameras 228a, 228b are optically aligned with each other in the optical detection assembly 200a, the cameras 228a-228d of the optical detection assembly 200c are similarly optically aligned with each other, such that any given pixels of the cameras 228a-228d have a known one-to-one correspondence with each other. That is, as illustrated in FIG. 23, the kth speckle grain of the sample light pattern 47 (after it is split into the first sample light pattern portion 47a) interferes with the reference light 42a with a phase of 0 to generate a kth speckle grain of the interference light pattern 48a that is detected by the kth pixel of the camera 228a; the same kth speckle grain of the sample light pattern 47 (after it is split into the first sample light pattern portion 47a) interferes with the reference light 42b with a phase of π to generate a kth speckle grain of the interference light pattern 48b that is detected by the kth pixel of the camera 228b; the same kth speckle grain of the sample light pattern 47 (after it is split into the second sample light pattern portion 47b) interferes with the reference light 42c with a phase of π/2 to generate a kth speckle grain of the interference light pattern 48c that is detected by the kth pixel of the camera 228c; and the same kth speckle grain of the sample light pattern 47 (after it is split into the second sample light pattern portion 47d) interferes with the reference light 42d with a phase of 3π/2 to generate a kth speckle grain of the interference light pattern 48d that is detected by the kth pixel of the camera 228d.

Since each kth pixel of the cameras 228a-228d have known correspondences via optical alignment, the quadruplet of intensity values detected by the kth pixels of the cameras 228a-228d serve as the analog to the four intensity values that would normally be stored by the kth pixel in the four bins of a lock-in camera.

At each corresponding quadruplet of kth pixels, the optical power received by the respective cameras 228a, 228b is equal to the summation of the power of the reference light 42a (P_{reference}A and P_{reference}B) input into the first beam splitter 258′, the sample light pattern 47 (PₛₐₘₚₗₑA and PₛₐₘₚₗₑB) input into the first beam splitter 258′, and an interference term between the reference light 42 and sample light pattern 47 (P_{interfere}A and P_{interfere}B); and the optical power received by the respective cameras 228c, 228d is equal to the summation of the power of the reference light 42b (P_{reference}C and P_{reference}D) input into the second beam splitter 258″, the sample light pattern 47 (PₛₐₘₚₗₑC and PₛₐₘₚₗₑD) input into the second beam splitter 258″, and an interference term between the reference light 42 and sample light pattern 47 (P_{interfere}C and P_{interfere}D). By the power conservation, the interference terms P_{interfere}A and P_{interfere}B are 180 degrees out of phase for cameras 228a, 228b, and the interference terms PᵢₙₜₑᵣᵣₑᵣₑC and P_{interfere}D are 180 degrees out of phase for cameras 228c, 228d.

It follows that, in the context of pulsed UOT, the power detected at a kth pixel for the first camera 228a can be expressed as:

Value_{1,k}=P_{background}+P_{signal}+P_{reference}+2(P_{signal}×P_{reference})^{1/2}×cos(φ_{unknown1,speckle k})+2(P_{signal}×P_{background})^{1/2}×cos(2π×fᵤₛ−φ_{unknown2,speckle k})+2(P_{reference}×P_{background})^{1/2}×cos(2π×fᵤₛ−φ_{unknown3,speckle k}); [22]

the power detected at the corresponding kth pixel of the second camera 228b can be expressed as:

Value_{2,k}=P_{background}+P_{signal}+P_{reference}+2(P_{signal}×P_{reference})^{1/2}×cos(φ_{unknown1,speckle k}+π)+2(P_{signal}×P_{background})^{1/2}×cos(2π×fᵤₛ−φ_{unknown2,speckle k}+π)+2(P_{reference}×P_{background})^{1/2}×cos(2π×fᵤₛ−φ_{unknown3,speckle k}+π); [23]

the power detected at the corresponding kth pixel of the second camera 228b can be expressed as:

Value_{3,k}=P_{background}+P_{signal}+P_{reference}+2(P_{signal}×P_{reference})^{1/2}×cos(φ_{unknown1,speckle k}+π/2)+2(P_{signal}×P_{background})^{1/2}×cos(2π×fᵤₛ−φ_{unknown2,speckle k}+π/2)+2(P_{reference}×P_{background})^{1/2}×cos(2π×fᵤₛ−φ_{unknown3,speckle k}+π/2); [24]

and
the power detected at the corresponding kth pixel of the second camera 228d can be expressed as:

Value_{4,k}=P_{background}+P_{signal}+P_{reference}+2(P_{signal}×P_{reference})^{1/2}×cos(φ_{unknown1,speckle k}+3π/2)+2(P_{signal}×P_{background})^{1/2}×cos(2π×fᵤₛ−φ_{unknown2,speckle k}+3π/2)+2(P_{reference}×P_{background})^{1/2}×cos(2π×fᵤₛ−φ_{unknown3,speckle k}+3π/2), [25]

where P_{background} represents light at frequency f+fᵤₛ that has not been tagged with the ultrasound 32; P_{signal} represents light at frequency f that has been tagged with the ultrasound 32; P_{reference} represents the reference light at frequency f; φ_{control} is the phase shift introduced into the reference light 42; and φ_{unknown1, speckle k}, φ_{unknown2, speckle k}, and φ_{unknown3, speckle k} are random phases at the kth speckle grain at the time of measurement, which originate via multiple scattering of coherent light inside the tissue.

The terms 2(P_{signal}×P_{background})^{1/2}×cos(2π×fᵤₛ−φ_{unknown2})+2(P_{reference}×P_{background})^{1/2}×cos(2π×fᵤₛ−φ_{unknown3}) oscillate at the frequency fᵤₛ, and are not detected by the cameras 228a, 228b, and thus, can be ignored. As such, equations [22]-[25] can be respectively reduced to:

Value_{l,k}=P_{background}+P_{signal}+P_{reference}+2(P_{signal}×P_{reference})^{1/2}×cos(φ_{unknown}); [26]

Value_{2,k}=P_{background}+P_{signal}+P_{reference}+2(P_{signal}×P_{reference})^{1/2}×cos(φ_{unknown}+π); [27]

and

Value_{3,k}=P_{background}+P_{signal}+P_{reference}+2(P_{signal}×P_{reference})^{1/2}×cos(φ_{unknown}+3π/2); [28]

Value_{4,k}=P_{background}+P_{signal}+P_{reference}+2(P_{signal}×P_{reference})^{1/2}×cos(φ_{unknown}+3π/2) [29].

These equations can be expressed as A+B×Cos(φ_{unknown}). Both the amplitude B (i.e., the interference term 2(P_{signal}×P_{reference})^{1/2}) and the unknown phase φ_{unknown} can be extracted by solving the resulting four equations using the standard trigonometric identities, and determining power of the signal light P_{signal} can be determined (either in the absolute sense or relative sense) from the extracted term interference term 2(P_{signal}×P_{reference}) using a known scaling relationship with the known or independently measurable power of the reference light P_{reference}.

As with the four-bin detection scheme described above with respect to the UOT system 10, with an N number of pixels, the signal-to-noise ratio (SNR) scales with N^{1/2} in accordance with equation [6] set forth above.

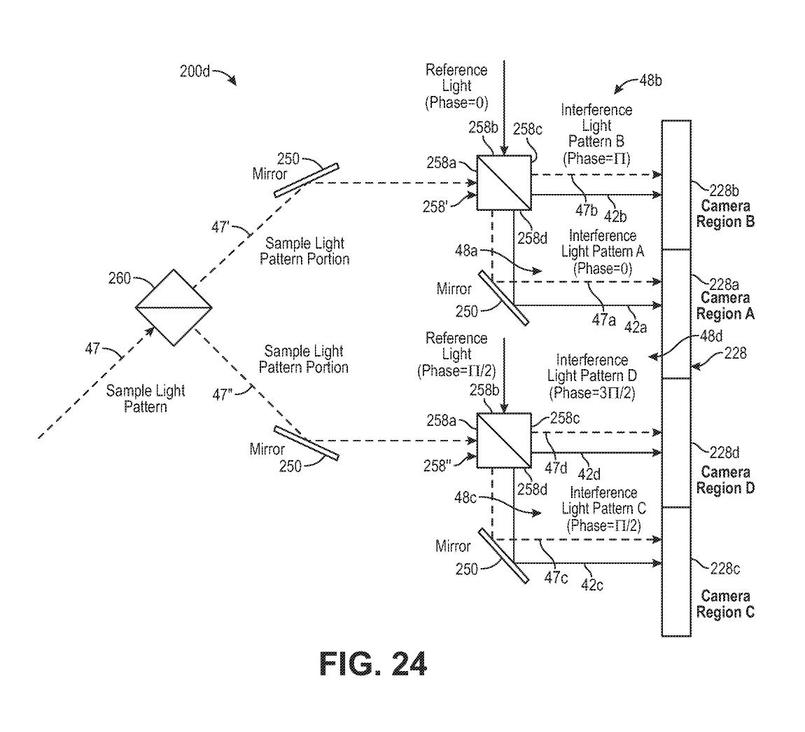

Although the optical detection assembly 200c has been described and illustrated as comprising detector arrays that take the form of separate cameras 228a-228d (in effect, the detector arrays are mechanically disposed on four separate camera microchips), an optical detection assembly 200d can comprise detection arrays that take the form of a single camera 228 (in effect, the four detector arrays are mechanically disposed on a single camera microchip), as illustrated in FIG. 24.

As with the optical detection assembly 200c, the beam splitter 260 of the optical detection assembly 200d splits the sample light pattern 47 into first and second sample light pattern portions 47′, 47″, and the first and second beam splitter/combiners 258′, 258″ of the optical detection assembly 200d split reference light 42 respectively into reference light 42 having an M number of different phase relative to the sample light pattern 47 (and in this case, reference light 42a-42d respectively having four different phases of 0, π, π/2, and 3n/2), and concurrently combining the sample light pattern portions 47a-47d and the reference light 42a-42d to respectively generate an M number of interference light patterns 48 (in this case, four interference light patterns 48a (“Interference Light Pattern A”), 48b (“Interference Light Pattern B”), 48c (“Interference Light Pattern C”), 48d (“Interference Light Pattern D”).

However, instead of respectively projecting the interference light patterns 48-48d onto four cameras, the beam splitter/combiners 258′, 258″ of the optical detection assembly 200d project the interference light patterns 48a-48d from their respective output ports onto four separate spatial regions 248a-248d of the same camera 228. To this end, the optical detection assembly 200d comprises two more angled mirrors 250 configured for respectively directing the interference light pattern 48a, 48c from the first output ports 258c of the first and second beam splitter/combiners 258′, 258″ toward the same plane to which the interference light patterns 48b, 48d are directed, i.e., towards the single camera 228.

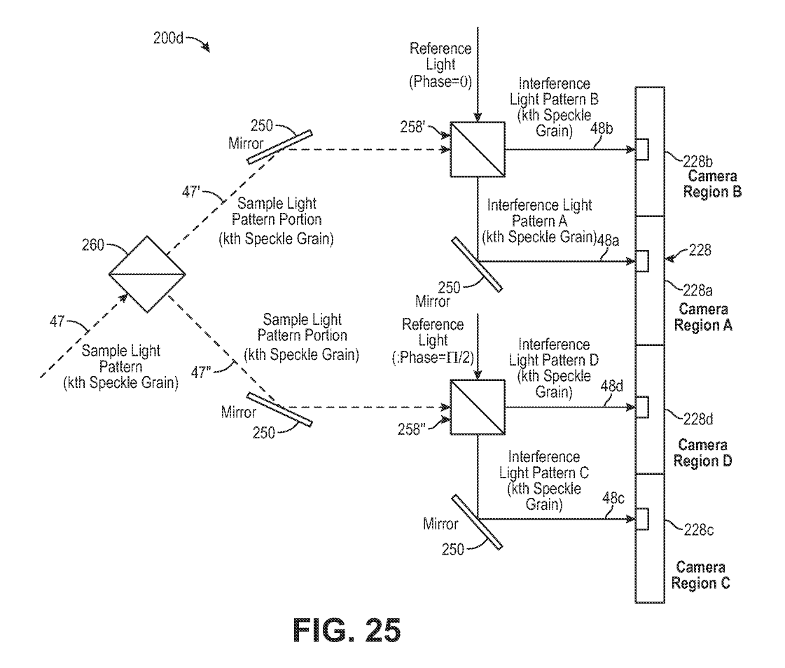

In the same manner as the cameras 228a-228d are optically aligned with each other in the optical detection assembly 200c, the spatial regions 228a-228d of the single camera 228 of the optical detection assembly 200d are optically aligned with each other, such that any given pixels on the spatial regions 228a-228d have a known one-to-one correspondence with each other. That is, as illustrated in FIG. 25, the kth speckle grain of the sample light pattern portion 47 (after being split into the first sample light pattern portion 47a) interferes with the reference light 42a with no phase shift (i.e., 0 phase) to generate a kth speckle grain of the interference light pattern 48a that is detected by the spatial region 228a of the camera 228; the same kth speckle grain of the sample light pattern 47 (after being split into the first sample light pattern portion 47a) interferes with the reference light 42b with a phase shift (i.e., π) to generate a corresponding kth speckle grain of the interference light pattern 48b that is detected by the kth pixel of the spatial region 228b of the camera 228; the same kth speckle grain of the sample light pattern 47 (after being split into the second sample light pattern portion 47b) interferes with the reference light 42c with a phase shift (i.e., π/2) to generate a corresponding kth speckle grain of the interference light pattern 48c that is detected by kth pixel of the spatial region 228c of the camera 228; and the same kth speckle grain of the sample light pattern 47 (after being split into the second sample light pattern portion 47b) interferes with the reference light 42d with a phase shift (i.e., 3π/2) to generate a corresponding kth speckle grain of the interference light pattern 48d that is detected by the kth pixel of the spatial region 228d of the camera 228.

The power of the signal light P_{signal} can be determined in accordance with equations [26]-[29], where Value_{1,k} is the power detected at a kth pixel for the first region 228a of the camera 228, Value_{2,k} is the power detected at a kth pixel for the second region 228b of the camera 228; Value_{3,k} is the power detected at a kth pixel for the second region 228c of the camera 228; and Value_{4,k} is the power detected at a kth pixel for the fourth region 228d of the camera 228.

Thus, it can be appreciated that the optical detection assemblies 200a-200d can be used in place of a conventional lock-in camera for UOT (or DOPC or snake photon OCT). In essence, the optical detection assemblies 200a-200d serve as drop-in replacements for conventional lock-in cameras. Instead of using two or more optical pulses that are interferometrically phase stepped and captured in separate lock-in camera data storing bins to perform a single “ultrasound tagged photon” quantification in UOT, the optical detection assemblies 200a-200d use a single optical pulse and a single camera frame for the same purpose. An arithmetic function (e.g., the absolute differencing and summation operations described above with respect to the optical detection assemblies 200a and 200b) can be performed on the arrays of values respectively detected by the arrays of pixels by a processor (e.g., the same processor used to determine the physiologically-dependent optical parameter of the target voxel or a separate circuit board, such as, e.g., an application specific integrated circuit board (ASIC) or a field-programmable field array (FPGA) customized to perform the processing). Alternatively, as will be described in further detail below, processing circuitry for performing the arithmetic function can be mechanically integrated into the camera microchip itself.

As discussed above, it is important that detector arrays be aligned pixel-by-pixel, whether the detector arrays are on separate cameras or spatial regions of a single camera. This can be accomplished, for instance, using image correlation methods and automated feedback find the optimal alignment configuration of the system. For example, a small bead may be measured on each of the detector arrays (cameras or regions of the camera), pixels that the bead appears on can be recorded, and this information can be stored in a look-up table to establish pixel-to-pixel correspondences. Alternatively, spatial light modulators (SLMs) may be used to project patterns onto each detector array, and pixels corresponding to defined parts of each pattern on each detector array may be stored in a look-up table to establish pixel-to-pixel correspondences. The cameras and any mirrors may be physically re-positioned during these calibration processes, e.g., using piezo-positioners or micro-electro-mechanical systems (MEMs) to physically establish a desired correspondence relationship between the pixels.

Although the UOT system 210 has been described as computing the intensity values Valueₖ of the speckle grains of each interference light pattern 48 in order to obtain the power of the light signal P_{signal}, it should be appreciated that the UOT system 10 may alternatively or optionally compute the random phases φ_{unknown} of the kth speckle grains of each interference light pattern 48 in order to obtain the phase across the wavefront of the light signal, e.g., for purposes of performing DOPC as described above, or for detecting optical phases in the target voxel 14 due to optical scatter changes by determining temporal changes in the wavefront phases of the signal light in accordance with equation [14] discussed above, wherein instead of using four data storing bins of a lock-in camera, four pixels of optically registered detector arrays (i.e., four different cameras or four different spatial regions of a single camera).

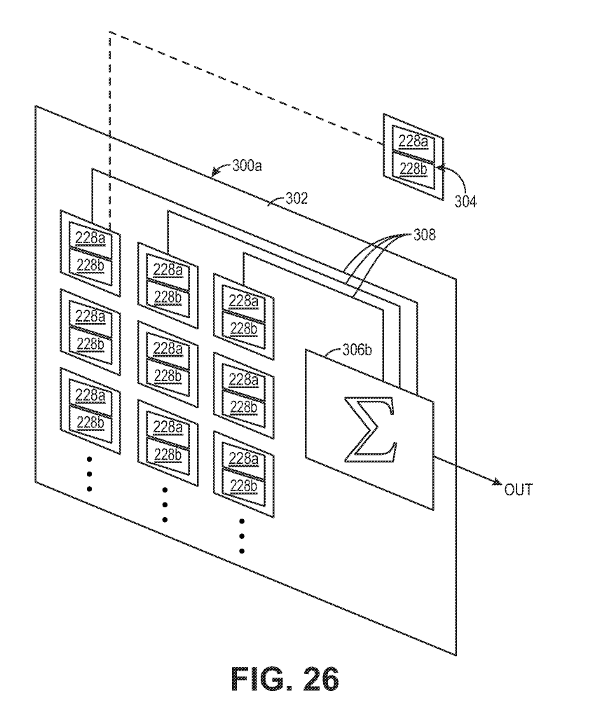

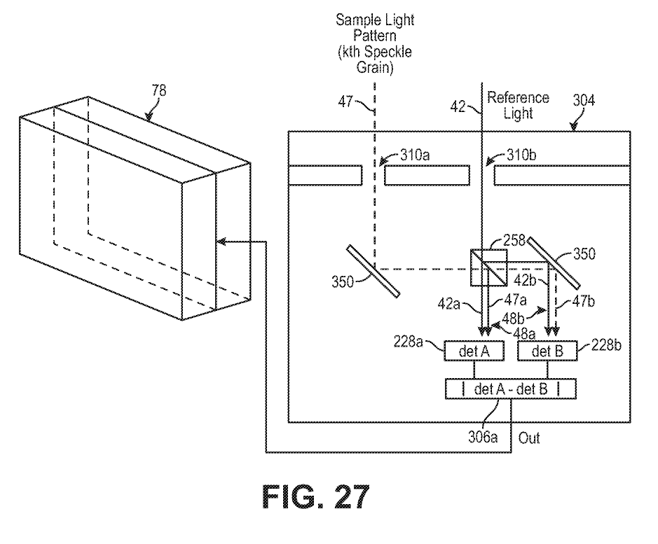

Referring now to FIG. 26, one embodiment of a camera microchip 300a that can be incorporated into an optical detection assembly comprises a substrate 302 and an array of N pixels 304, each of which contains miniaturized, integrated micro-optics, such that light splitting and combining function and interference pattern detection functions are performed at the pixel-level, as illustrated in FIG. 27, as described in further detail below. The camera microchip 300a essentially is a custom opto-electronic camera that performs interferometry at a pixel level.

To this end, the camera microchip 300a comprises an M number of optically registered detector arrays 228 mechanically integrated within the substrate 302 (and in this case, two detector arrays 228a, 228b). In the illustrated embodiment, the two detector arrays 228a, 228b are interlaced with each other, such that corresponding detectors of the arrays 228a, 228b are arranged as closely spaced side-by-side detector pairs (i.e., each detector pair comprises two corresponding detectors 228a, 228b), each of which represents a pixel 304 of the camera microchip 300a. Thus, the camera microchip 300a comprises an array of pixels 304, each comprising two corresponding detectors 228a, 228b. In the same manner described above with respect to the optical detection assembly 200d, the detector array 228a is configured for detecting an N number of spatial components of the interference light pattern 48a (one spatial component per pixel 304), and outputting a respective N number of intensity values for the respective spatial components (one intensity value “det A” shown in FIG. 27); and the detector array 228b is configured for detecting an N number of spatial components of the interference light pattern 48b (one spatial component per pixel 304), and outputting a respective N number of intensity values for the respective spatial components (one intensity value “det B” shown in FIG. 27).

The camera 300a further comprises processing circuitry 306 integrated within the substrate 302 for performing the arithmetic function on the respective intensity values respectively output by pixels containing corresponding detectors of the respective detector arrays 228a, 228b, and in this case, computing the sum of the absolute differences between the corresponding kth intensity values of each pixel. In the illustrated embodiment, local processing circuitry 306a (shown in FIG. 27) is distributed amongst the pixels 302, such that the absolute difference of corresponding intensity values (|det A−det B|) is computed locally at each respective pixel 302, and processing centralized circuitry 306b is remote from the detector arrays 228a, 228b for computing the sum of these absolute differences, and outputting a single value representative of the P_{signal} in equations [20] or [21]. Any averaging of the absolute difference summing function, if necessary, can be conveniently and quickly performed off-chip as a single computation having one input value and one output value. The outputs of the local processing circuitry 306a are coupled to respective inputs of the remote processing circuitry 306b via a respective N number of electrically conductive traces 308. Thus, the remote processing circuitry 306b comprises an N number of inputs, and one output for one value (i.e., P_{signal}).

It should be noted that by localizing a portion of the processing circuitry with the individual pixels, the number of electrically conductive traces 308 is minimized. However, the absolute difference summing function can be alternatively performed in the remote processing circuitry 306b, in which case, an increased number of electrically conductive traces 308 would be required to connect the outputs of the individual detectors to the processing circuitry 306b.

The camera microchip 300a further comprises an array of beam splitter/combiners 258, an array of sample input ports 310a, and an array of reference input ports 310b integrated within the substrate 302, such that, as illustrated in FIG. 27, each pixel 302 has a dedicated splitter/combiner 258, a dedicated sample input port 310a, a dedicated reference input port 310b, and a dedicated pair of corresponding detectors 228a, 228b (one from the detector array 228a, and the other from the detector array 228b).

The array of sample input ports 310a is configured for respectively receiving the sample light pattern 47, and the array of reference input ports 310b is configured for receiving the reference light 42. In the same manner described above with respect to the optical detection assembly 200a, the array of beam splitter/combiners 258 is configured for splitting the reference light 42 into reference light 42a, 42b respectively having two different phases of 0 and π, splitting the spatial component of the sample light pattern 47 respectively into the sample light pattern portions 47a, 47b, and concurrently combining the sample light portions 47a, 47b and the reference light 42a, 42b to respectively generate two interference light patterns 48a, 48b. However, in this case, the camera microchip 300a locally performs this entire process on a pixel-by-pixel basis.

Thus, for each pixel, a spatial component of the sample light pattern 47 enters the sample input port 310a, where it is split by the beam splitter/combiner 258 into a reflected spatial component of a sample pattern portion 47a and a transmitted spatial component of a sample light pattern portion 47b, and the reference light 42 enters the reference input port 310b, where it is split by the beam splitter/combiner 258 into transmitted reference light 42a and reflected reference light 42b. In a simultaneous manner, the reflected spatial component of the sample light pattern portion 47a interferes with the transmitted reference light 42a to generate a respective spatial component of the interference light pattern 48a, and the transmitted spatial component of the sample light pattern portion 47b interferes with the reflected reference light 42b to generate a respective spatial component of the interference light pattern 48a.

In the illustrated embodiment, the beam splitter/combiner 258 is in the direct linear path of the sample input port 310a, but laterally offset from the direct linear path of the reference input port 310b. In this case, the camera microchip 300 comprises an angled mirror 250 configured for redirecting the reference light 47 from the reference input port 310b towards the beam splitter/combiner 258. Alternatively, if the beam splitter/combiner 258 is in the direct linear path of the reference input port 310b, but laterally offset from the direct linear path of the sample input port 310a, the angled mirror 350 can instead be configured for redirecting the spatial component of the sample light pattern 47 from the sample input port 310a towards the beam splitter/combiner 258.

For each pixel, the detectors 228a, 228b are configured for concurrently detecting the respective spatial components of the two interference light patterns 48a, 48b, and generating two values representative of intensities of the two respective spatial components of the interference light patterns 48a, 48b. Thus, the sample light field 47 and reference light 42 combine to project the respective spatial component of the interference light pattern 48a onto the detector 228a, and likewise to project the respective spatial component of the interference light pattern 48b onto the detector 228b, but with respect to a different phase of the reference light 42.

As with the optical detection assembly 200a described above, the detectors 228a, 228b are optically aligned with each other, such that they have a known one-to-one correspondence with each other for that pixel. That is, a spatial component (“speckle k,” indicating the kth speckle grain of the speckle light field) of the sample light pattern 47 interferes with the reference light 42a with no phase shift (i.e., 0) to generate a kth speckle grain of the interference light pattern 48a that is detected by the detector 228a, and the same kth speckle grain of the sample light pattern 47 interferes with the reference light 42b with a phase shift (i.e., π) to generate a corresponding kth speckle grain of the interference light pattern 48b that is detected by the detector 228b. Thus, since the detectors 228a, 228b, in effect, are positioned below the same pixel and hence the same speckle grain, the pair of intensity values outputted by the detectors 228a, 228b serve as the analog to the pair of intensity values that would normally be stored by the kth pixel in the two data storing bins of a lock-in camera. It should be appreciated that although FIG. 27 illustrates one speckle “k,” an equivalent process for measuring the speckle k takes place for all speckles k in parallel in the manner of imaging an entire speckle light field. It should be appreciated that auto-alignment mechanisms are not necessary to optically align the detectors 228a, 228b, since they are integrated within one pixel.

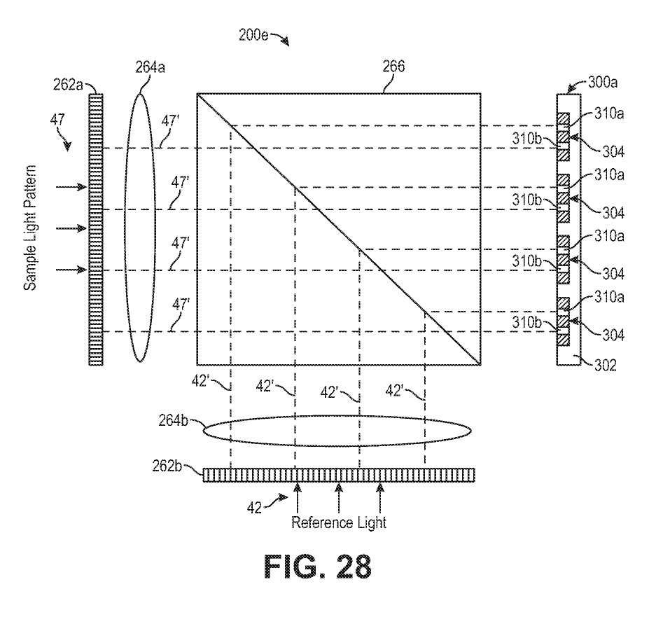

However, it is important that the spatial components of the sample light pattern 47 be respectively optically aligned with the sample input ports 310a, and that the reference light 40 only enter the reference input ports 310b. To this end, and with reference to FIG. 28, the camera microchip 300 of FIGS. 26 and 27 can be incorporated into an optical detection assembly 200e that additionally comprises a first grate 262a configured for transforming the sample light pattern 47 into an array of sample light pattern beamlets 47′ (corresponding to the spatial components), a first lens 264a configured for focusing the array of reference beamlets 47′ into the array of reference input ports 310a, a second grate 262b configured for transforming the reference light 42 into an array of reference light beamlets 42′, and a second lens 264b configured for focusing the array of reference light beamlets 42′ into the array of reference input ports 310b. The optical detection assembly 200e may further comprise a light combiner 266 that transmits the focused sample light pattern beamlets 42′ from the first lens 264a into the sample input ports 310a on the camera microchip 300, and that reflects the focused reference light pattern beamlets 47′ from the second lens 264b into the reference input ports 310b on the camera microchip 300a, or alternatively, vice versa.

Thus, the optical detection assembly 200e ensures that the kth speckle grain of the sample light pattern 47 enters the sample input port 310a of the kth pixel 302, while avoiding the reference input port 310b, and likewise, ensures that the reference light 42 enters the reference input ports 310a while avoiding the sample input ports 310a.

Referring now to FIG. 28, another embodiment of a camera microchip 300b that can be incorporated into an optical detection assembly for performing DOPC will be described. The camera microchip 300b is similar to the camera microchip 300a described above, except that it has an integrated camera-spatial-light-modulator architecture that performs phase conjugation using an SLM array 78 to perform optical phase conjugation of the signal light 44 in a digital optical phase conjugation (DOPC) technique in the same manner described above with respect to FIGS. 9a-9b and 10a-10b, i.e., to boost the sensitivity of the pulsed UOT detection by extracting the phase of the signal light 44 in the pulsed UOT detection scheme, computing the phase conjugate of the wavefront of the signal light 44, and feeding it back through an SLM array 78 to achieve enhanced light focusing at the target voxel 14. The pixels 302 of the camera microchip 300b are co-registered (e.g., pixel-by-pixel) with the SLM array 78, i.e., in direct alignment with the pixels of the SLM array 78, such that the outputs of the local processing circuitry 306a are coupled to respective inputs of the pixels of the SLM array 78. The SLM array 78 may be built directly on top of the detector array 228 or detector arrays 228 to create a phase conjugation array.

Furthermore, the remote processing circuitry 306b is not required, and therefore not included within the camera microchip 300b, for summing the absolute difference values output by the array of pixels 302. Instead, like the camera microchip 300a, the camera microchip 300b only includes local processing circuitry 306a is distributed amongst the pixels, such that the absolute difference of corresponding intensity values is computed locally at each respective pixel 302.

Notably, the increased processing speed enabled by the local data processing architecture of the camera microchip 300b lends itself well to DOPC detection, which additionally requires phase conjugation computation that would otherwise be slowed if data transfer and off-chip computation were required. Thus, phase conjugation can be performed directly on-chip without the need to shuttle information off-chip or to perform off-chip computation, thereby enabling extremely rapid phase conjugation. Because the camera microchip 300b can perform phase conjugation very rapidly, must faster than the speckle decorrelation time, even for imaging many centimeters inside a dynamic, strongly scattering biological specimen, such as the human brain, it would enable scattering correction methods, such as DOPC, to be performed for focusing light many centimeters inside such dynamic medium, effectively overcoming the limit on DOPC depth currently posed by speckle decorrelation.

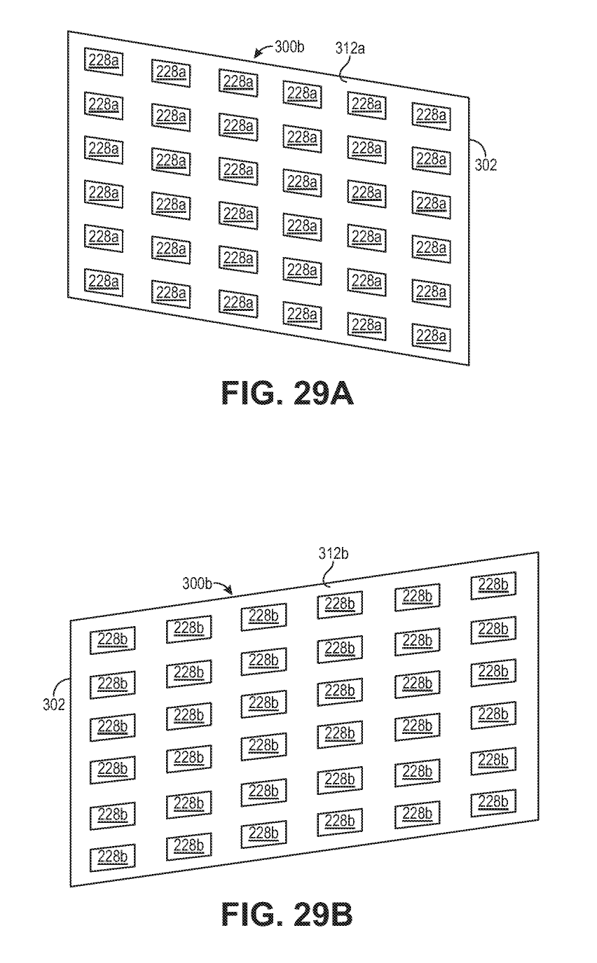

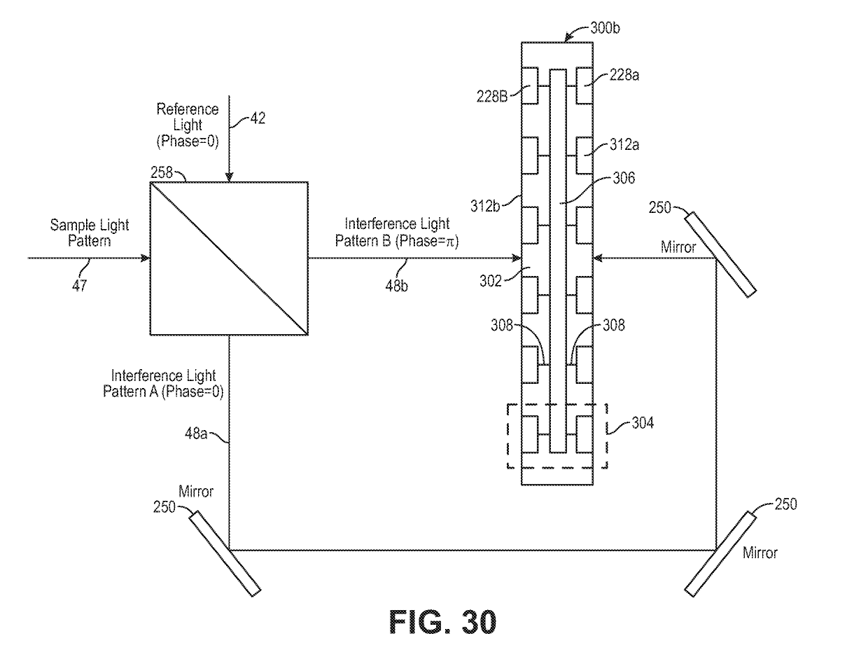

Referring now to FIGS. 29a, 29b, and 30, another embodiment of a camera microchip 300b that provides a simplified design that does not require miniaturized, integrated micro-optics that perform the light splitting and combining functions at the pixel-level, but rather is designed to be used with a single off-chip beam splitter/combiner 258 as part of an optical detection assembly 200f.

Like the camera microchip 300a, the camera microchip 300b comprises a substrate 302 and two optically registered detector arrays 228a, 228b mechanically integrated within the substrate 302 to form detector pairs, each of which comprises two corresponding detectors 228a, 228b to form a pixel 304. However, unlike the camera microchip 300a, the detector arrays 228a, 228b are not interlaced with each other, but rather are respectively disposed on opposite sides 312a, 312b of the substrate 302 to form detector pairs, each of which comprises two corresponding detectors 228a, 228b to form the pixel 304. Thus, the camera microchip 300b comprises an N number of pixels 304, each comprising two corresponding detectors 228a, 228b.

In the same manner described above with respect to the optical detection assembly 200d, the detector array 228a is configured for detecting an N number of spatial components of the interference light pattern 48a, and outputting a respective N number of intensity values for the respective spatial components, and the detector array 228b is configured for detecting an N number of spatial components of the interference light pattern 48b, and outputting a respective N number of intensity values for the respective spatial components.

However, in this case, the interference light patterns 48a, 48b are projected onto the detector arrays 228a, 228b on opposite sides of the substrate 302. As with the optical detection assembly 200a, the beam splitter/combiner 258 is configured for splitting the reference light 42 respectively into reference light 42a, 42b respectively having two different phases of 0 and π, splitting the sample light pattern 47 respectively into sample light pattern portions 47a, 47b, and concurrently combining the sample light pattern portions 47a, 47b and the reference light 42a, 42b to respectively generate two interference light patterns 48a, 48b.

The beam splitter/combiner 258 projects the interference light pattern 48a directly onto the detector array 228a on one side of the substrate 302 of the camera microchip 300b. The optical detection assembly 200f comprises angled mirrors 250 configured for redirecting the interference light pattern 48a from the beam splitter/combiner 258 towards the detector array 228a on the opposite side 312a of the substrate 302 of the camera microchip 300b.

As best shown in FIG. 30, like the camera microchip 300a, the camera microchip 300b further comprises processing circuitry 306 integrated within the substrate 302 for performing the arithmetic function on the respective intensity values respectively output by pixels containing corresponding detectors of the respective detector arrays 228a, 228b, and in this case, computing the sum of the absolute differences between the corresponding kth intensity values of each pixel and outputting a single value representative of P_{signal} in equations [20] or [21]. The outputs of the local processing circuitry 306a are coupled to respective inputs of the remote processing circuitry 306b via a respective N number of electrically conductive traces 308. Thus, the remote processing circuitry 306b comprises an N number of inputs, and one output for one value (i.e., P_{signal}). As can be appreciated the processing circuitry 306 can be conveniently located between the respective detector arrays 228a, 228b, thereby providing an efficient and simplistic means of routing signals from the detector arrays 228a, 228b to the processing circuitry 306. Moreover, any average of the absolute value of the difference or other computation can be performed through the processing circuitry 306 (which may be implemented by local analog and/or mixed-signal circuitry) in the space between the two detector arrays 228a, 228b, minimizing the amount of long-range signal routing and the power consumption associated with analog to digital conversion and other operations, and thus making the system more space and power efficient as it scales to high pixel counts.

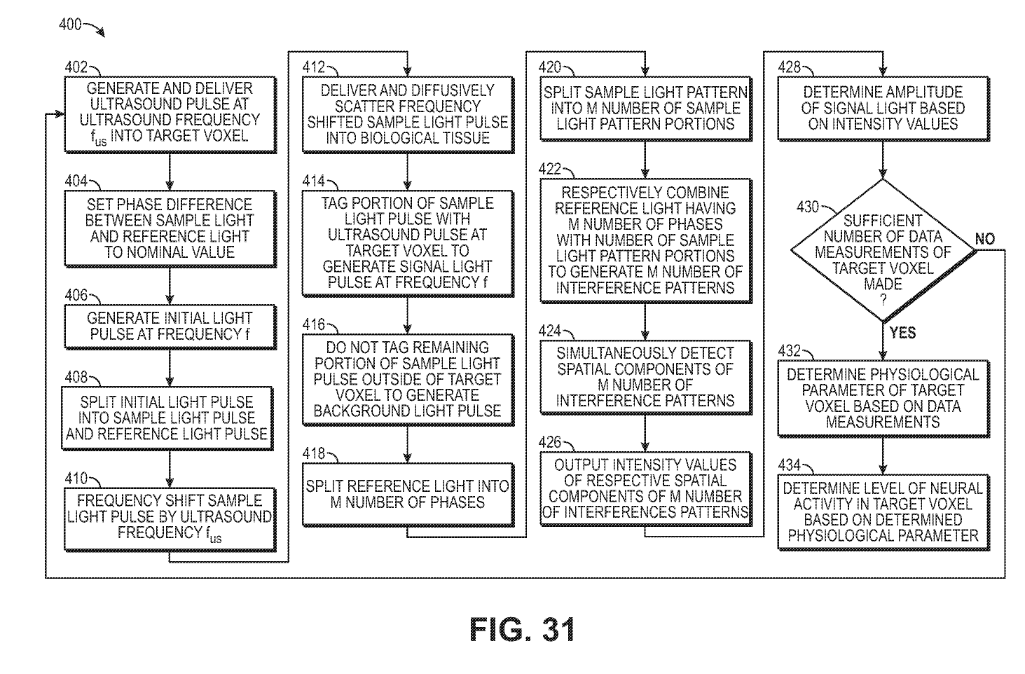

Referring to FIG. 31, one particular method 400 performed by the UOT system 210 to non-invasively image the target voxel 14 in anatomical structure 16 will now be described.

The controller 24 operates the acoustic assembly 20 to generate and deliver a pulse of ultrasound 32 having a frequency fᵤₛ into the anatomical structure 16, e.g., by sending a control signal to the signal generator 36 to pulse an electrical signal on and off (step 402). The controller 24 sets the phase difference between the sample light 40 and the reference light 42 to a nominal value (e.g., 0) (step 404). Next, the controller 24 operates the interferometer 222 to generate and emit a pulse of source light 38 having a frequency f, e.g., by sending a control signal to the drive circuit to pulse the light source 50 on and off (step 406). The interferometer 222 (e.g., via the beam splitter 52) splits the pulse of source light 38 into a pulse of sample light 40 and a pulse of reference light 42 (step 408).

The wavelength (and thus, the frequency f) of the source light 38 may be selected based on the physiologically-dependent optical parameter to be ultimately determined. For example, if the physiologically-dependent optical parameter is the level of deoxygenated and/or oxygenated hemoglobin concentration or relative abundance, the wavelength of the source light 38 may be in the range of 605 nanometers to 950 nanometers, whereas if the physiologically-dependent optical parameter to be determined is a water absorption level (level of water concentration or relative water concentration), the wavelength of the source light 38 may be in the range of 950-1080 nanometers.

Next, prior to the pulse of sample light 40 entering the anatomical structure 16, the controller 24 operates the interferometer 222 to frequency shift the pulse of sample light 40 by the ultrasound frequency fᵤₛe.g., by sending a control signal to the frequency shifter 56, resulting in the pulse of sample light 40 having a frequency f−fᵤₛ (step 410). It should be appreciated that, although this frequency shifting technique implements the frequency shifting technique illustrated in FIG. 3a, other frequency shifting techniques can be utilized, such as those illustrated in FIGS. 3b-3d. The frequency-shifted pulse of sample light 40 is then delivered into and diffusively scattered within the anatomical structure 16 (step 412). As the pulse of frequency shifted sample light 40 scatters diffusively through the anatomical structure 16, a portion will pass through the target voxel 14 and be frequency shifted (i.e., tagged) back to its original frequency f by the pulse of ultrasound 32 passing through the target voxel 14, resulting in a pulse of scattered signal light 44 having the same frequency f (step 414); and remaining portion will not pass through the target voxel 14, and thus will not be frequency shifted by the pulse of ultrasound 32, resulting in a pulse of scattered background light 46 having a frequency f−fᵤₛ (the same frequency as the frequency shifted sample light 40 prior to entering the anatomical structure 16) (step 416).

Next, the interferometer 222 then concurrently, via the one or more beam splitter/combiners 258, splits the reference light 42 respectively into an M number of different phases (step 418), splits the sample light pattern 47 into an M number of sample light pattern portions (step 420), and respectively combines the M number of sample light pattern portions and the reference light 42 to respectively generate the M number of interference light patterns 48 (step 422).

Then, under control of the controller 24, each of M number of detector arrays 228 simultaneously detect respective spatial components of the corresponding interference light pattern 48 (i.e., speckle grains in the case where the interference light pattern includes a speckle light pattern) (step 424), and output intensity values for the respective spatial components of the N number of interference light patterns 48 (step 426). The processor (e.g., a portion of which may include processing circuitry 306) then determines the amplitude of the signal light 44 based on these intensity values (e.g., using equations [20] or [21] if M equal two, and using equations [26]-[29] if M equals four) (step 428).

Steps 402-428 can be iterated to repeatedly acquire data measurements of the target voxel 14, and if a sufficient number of data measurements have been acquired (step 430), the processor 30 may then determine the physiologically-dependent optical parameter (e.g., level of deoxygenated and/or oxygenated hemoglobin concentration or relative abundance or level of water concentration or relative water concentration) of the target voxel 14 based on the data measurements (step 432). In the case where the target voxel 14 is brain matter, the processor 30 may further determine the level of neural activity within the target voxel 14 based on the determined physiologically-dependent optical parameter (step 434), e.g., in the manner described above with respect to step 134 of the method 100 illustrated in FIG. 13.

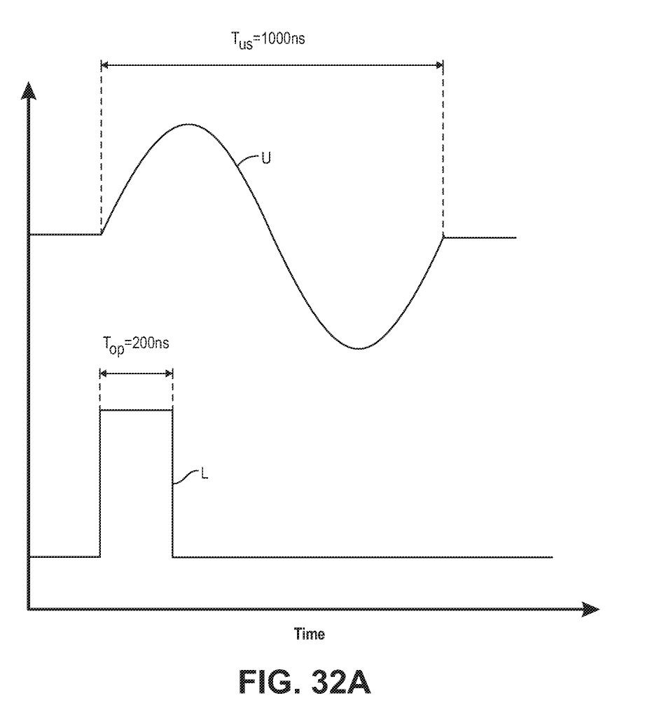

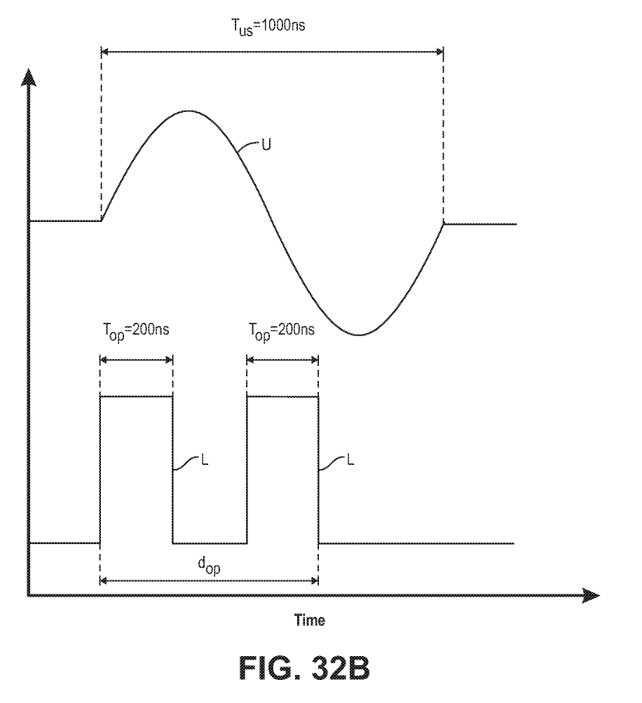

In one particular advantageous implementation of an interferometer that can be used in the UOT system 210 of FIG. 15, the duration Tₒₚ of the sample light pulse L is selected to be less than the duration Tᵤₛ of the ultrasound pulse U, as shown in FIG. 32a. In this particular example, the light pulse duration Tₒₚ is selected to be 200 ns, whereas the ultrasound pulse duration Tᵤₛ is selected to be 1000 ns (equivalent to 1 MHz for a single cycle per pulse). The shutters of the cameras may be opened longer, as may be the exposure time, but only during the sample light pulse L do photons impinge on the image sensors of the cameras. In some instances, it might be desirable to use multiple sample light pulses L, as illustrated in FIG. 32b, to provide additional photo-electrons to the camera(s); however, any series of sample light pulses L should have a combined duration dₒₚ less than the duration Tᵤₛ of the ultrasound pulse U, and be completed within the speckle decorrelation time. Whether the sample light 40 has a single light pulse L or multiple light pulses L per ultrasound pulse U, the combined duration dₒₚ may be less than 1000 ns, e.g., equal to or less than 500 ns or even equal to or less than 200 ns. Ultimately, the ultrasound pulse duration Tᵤₛ depends on the ultrasound frequency fᵤₛ used to tag the signal light 44 (shown in FIG. 15). A higher ultrasound frequency fᵤₛ results in a better spatial resolution, but also results in a higher attenuation in tissue, resulting in more heating of the tissue and lower power at the target voxel 14 (shown in FIG. 15).

Notably, the selection of a relatively short light pulse duration Tₒₚ is advantageous in that it minimizes the complexity and cost of the pulsed laser source. For example, a conventional off-the-shelf pulsed laser source (e.g., a commercially available dye laser) may be used for light pulse durations Tₒₚ of 200 ns or less. The selection of a relatively short light combined duration dₒₚ for the light pulse(s) L also allows imaging at depths corresponding to shorter speckle decorrelation times, improving maximum achievable imaging depth, since decorrelation time scales super-linearly with the depth of the tissue to be imaged, as briefly discussed above.

Furthermore, the selection of a relatively short light combined duration dₒₚ for the light pulse(s) L also corresponds to an improved axial resolution (i.e., the depth resolution or ultrasound propagation direction), since the ultrasound pulse U moves a shorter distance during the duration of the sample light pulse(s) L, minimizing any blurring effect caused by such movement. In general, the theoretical z-resolution of the UOT system 210 of FIG. 15 can be expressed as:

Z=τᵤₛ*(N+dₒₚ/Tᵤₛ), [30]

where Z is the axial resolution, τᵤₛ is the wavelength of the ultrasound, N is the number of cycles in the ultrasound pulse U, dₒₚ is the combined duration of the sample light pulse(s) L (light pulse duration Tₒₚ in the case of a single optical pulse L) and Tᵤₛ is the duration of the ultrasound pulse U.

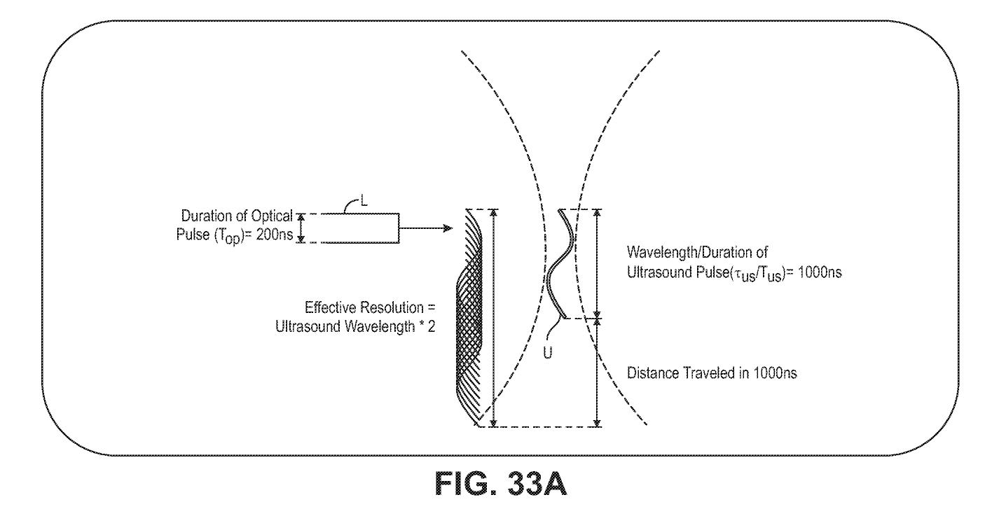

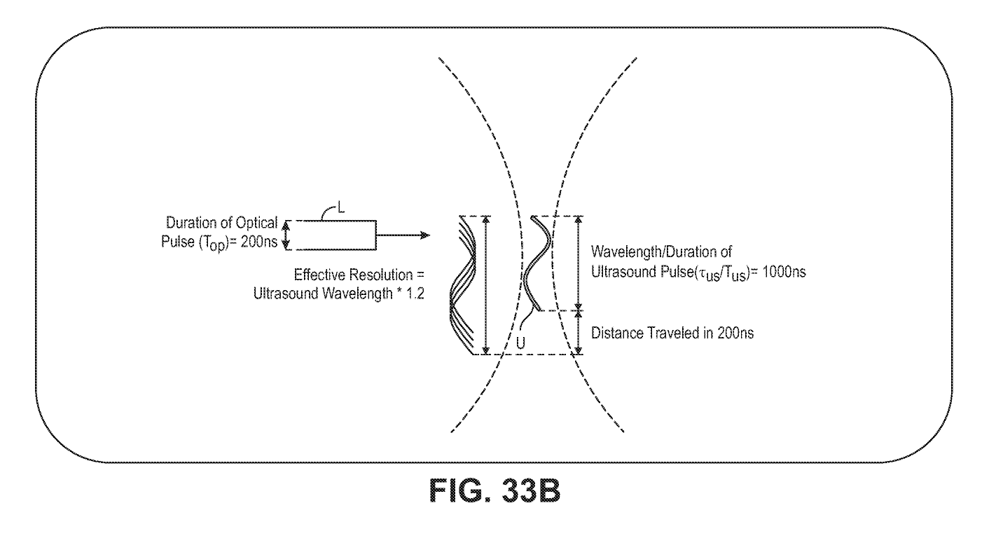

Thus, for a single ultrasound pulse U having a wavelength τᵤₛ of 1.5 mm (equivalent to 1 MHz ultrasound frequency) and a duration Tᵤₛ of 1000 ns, and a single corresponding light pulse L having a matching duration Tₒₚ of 1000 ns, the axial resolution Z will be equal to 1.5 mm*(1+1000/1000)=1.5 mm*2=3 mm, represented by the 1.5 mm wavelength of the ultrasound pulse U and an additional 1.5 mm distance that the ultrasound pulse U traveled in 1000 ns, as illustrated in FIG. 33a. In contrast, for the same single ultrasound pulse U, but a corresponding single light pulse L having a duration τₒₚ of 200 ns, the axial resolution Z is equal to 1.5 mm*(1+200/1000)=1.5 mm*1.2=1.8 mm, represented by the 1.5 mm wavelength of the ultrasound pulse U and an additional 1.5 mm distance that the ultrasound pulse U traveled in 200 ns, as illustrated in FIG. 33b. As can be appreciated by FIGS. 33a and 33b, matching the duration of the light pulse duration Tₒₚ with the ultrasound pulse duration Tᵤₛ results in a relatively blurred axial resolution, whereas decreasing the light pulse duration Tₒₚ relative to the light pulse duration Tᵤₛ results in a sharper axial resolution (by 1.2 mm or 40%).

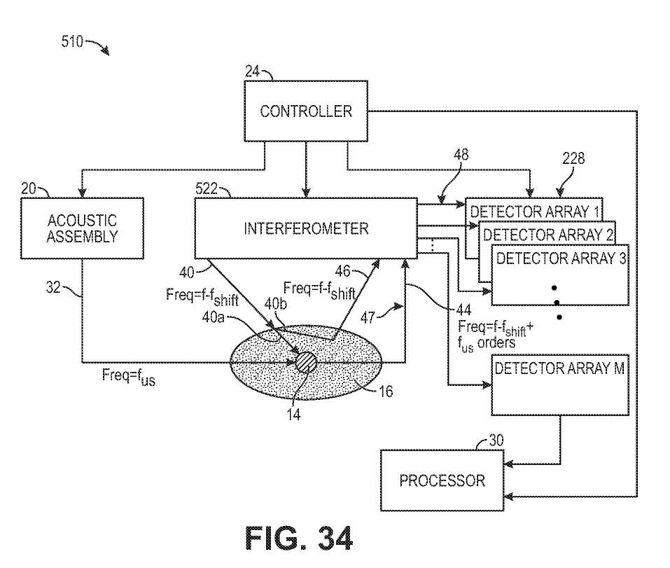

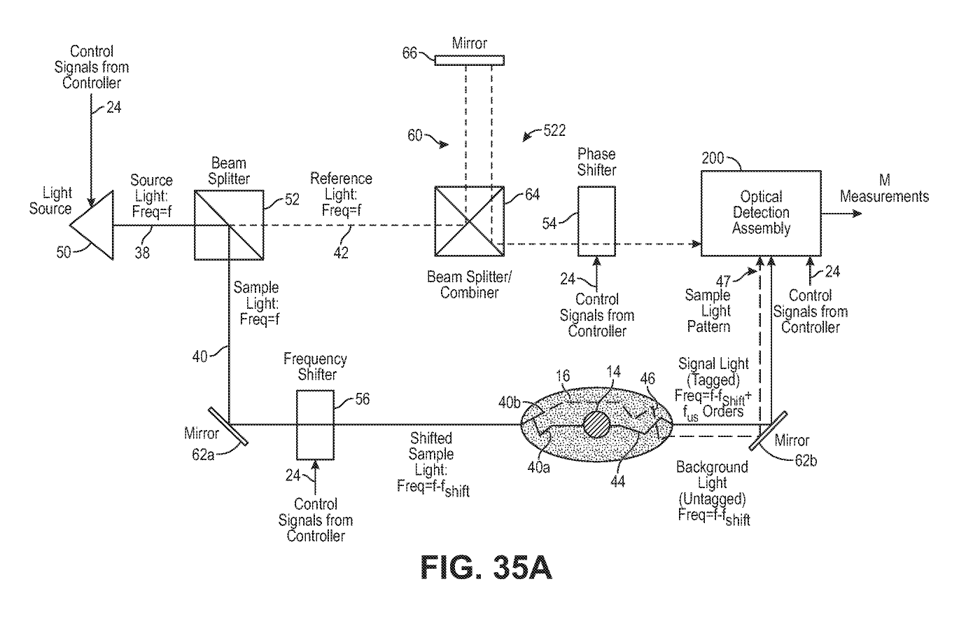

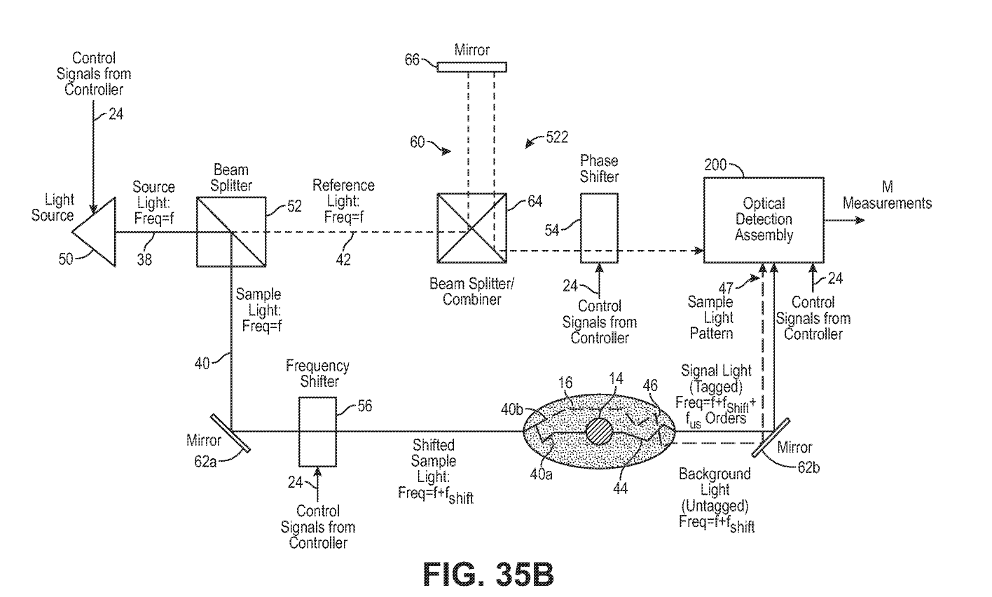

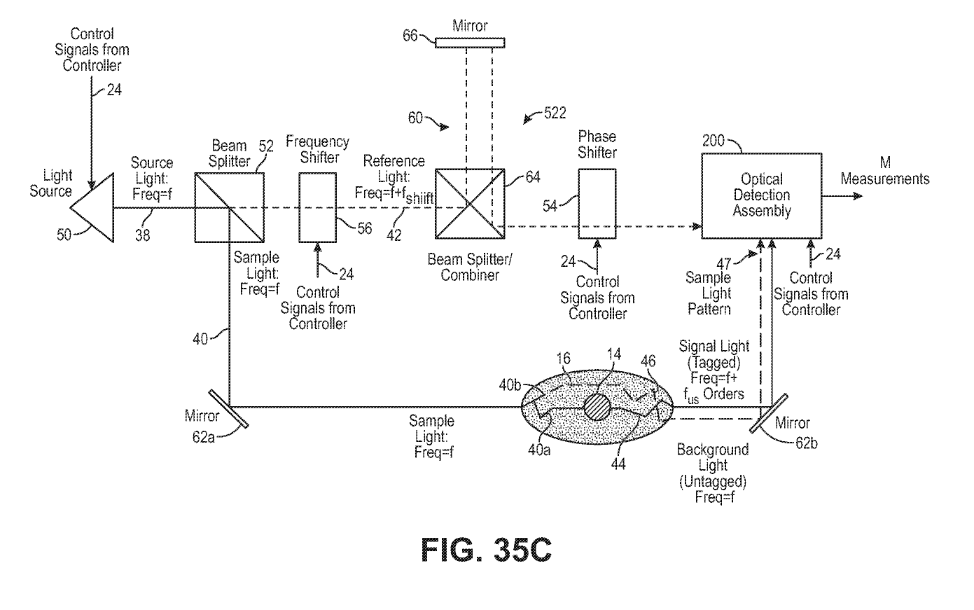

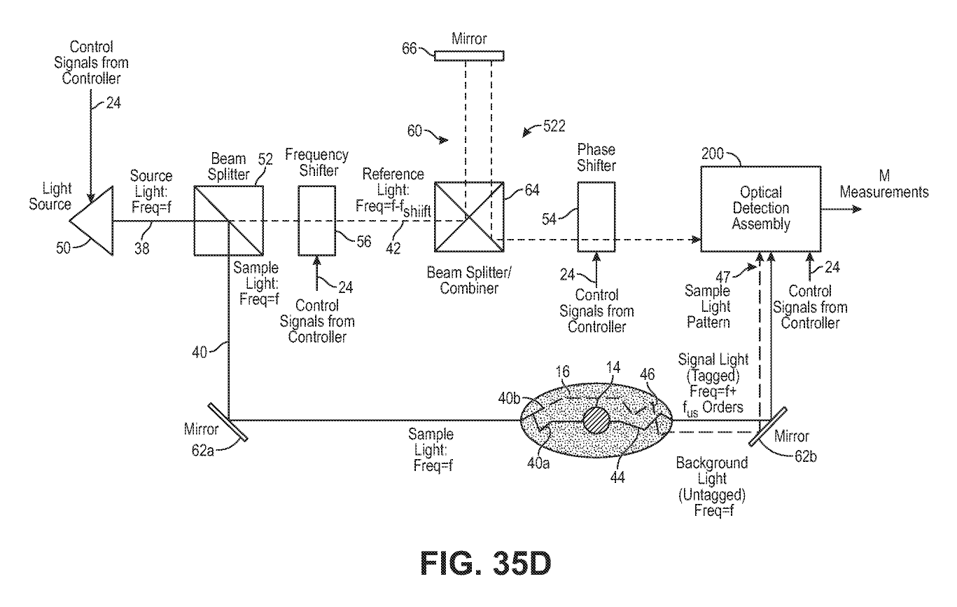

It should be noted that the signal detection efficiency of the UOT system 210 of FIG. 15, when using a relatively short combined duration dₒₚ for the light pulse(s) L (light pulse duration Tₒₚ in the case of a single light pulse L) will be decreased. However, one embodiment of a UOT system 510 illustrated in FIG. 34 is designed to combine the reference light 42 and the signal light 44 in a heterodyne manner in order to maximize the signal detection efficiency. In particular, the UOT system 510 is similar to the UOT system 210 illustrated in FIG. 15, with the exception that the UOT system 510 comprises an interferometer 522 configured for shifting the frequency f of the sample light 40 and the reference light 42 (shown in FIGS. 35a-35d) relative to each other by a frequency f_{shift} different from (rather than equal to) the frequency fᵤₛ of the ultrasound 32, such that, upon combining by the interferometer 222, the frequency of the ultrasound tagged signal light 44 will be shifted to a different frequency as the reference light 42.

That is, the sample light portion 40a passing through the target voxel 14 will be frequency shifted (i.e., tagged) by positive and negative multiples of the ultrasound frequency fᵤₛ (due to Raman-Nath diffraction and movement of scattering centers) of ultrasound 32 that also passes through the target voxel 14, such that the frequency of the signal light 44 differs from the frequency of the reference light 42 by f_{shift}+_{fits} (+1^{st} order), f_{shift}−fᵤₛ (−1^{st} order), f_{shift}+2fᵤₛ (+2^{nd} order), f_{shift}−2fᵤₛ (−2^{nd} order), etc. As a result, interference light patterns 48 having different ordered terms will be generated from the combination of the sample light pattern 47 and the reference light 42, as will be described in further detail below.

In the embodiment illustrated in FIG. 34, the interferometer 522 down frequency shifts the sample light 40 by the frequency f_{shift}, such that the ultrasound tagged signal light 44 has the frequencies f−f_{shift}+fᵤₛ (1^{st} order), f−f_{shift}−fᵤₛ (1^{st} order), f−f_{shift}+2fᵤₛ (2^{nd} order), f−f_{shift}−2fᵤₛ (2^{nd} order), etc.; the untagged background light 46 has the frequency f−f_{shift}; and the reference light 42 has a frequency f, thereby enabling combination of the reference light 42 and signal light 44 in a heterodyne manner, as better illustrated in interferometer 222 of FIG. 35a. However, the interferometer 522 may up frequency shift the sample light 40 by the frequency f_{shift}, such that, the ultrasound tagged signal light 44 has the frequencies f+f_{shift}+fᵤₛ (1^{st} order) f+f_{shift}−fᵤₛ (1^{st} order), f+f_{shift}+2fᵤₛ (2^{nd} order), f+f_{shift}-2fᵤₛ (2^{nd} order), etc., the untagged background light 46 has the frequency f+f_{shift}, and the reference light 42 has a frequency f (see FIG. 35b); may up frequency shift the reference light 42 by the frequency f_{shift}, such that the ultrasound tagged signal light 44 has the frequencies f+fᵤₛ (1^{st} order), f−fᵤₛ (1^{st} order), f+2fᵤₛ (2^{nd} order), f−2fᵤₛ (2^{nd} order), etc., the untagged background light 46 has the frequency f, and the reference light 42 has a frequency f+f_{shift} (see FIG. 35c); may down frequency shift the reference light 42 by the frequency f_{shift}, such that the ultrasound tagged signal light 44 has the frequencies f+fᵤₛ (1^{st} order), f−fᵤₛ (1^{st} order), f+2fᵤₛ (2^{nd} order), f−2fᵤₛ (2^{nd} order), etc., the untagged background light 46 has the frequency f, and the reference light 42 has a frequency f−f_{shift} (see FIG. 35d); or perform any other frequency shift of the sample light 40 or reference light 42 that results in the heterodyne combination of the reference light 42 and the signal light 44.

Significantly, assuming a rectangular pulsed waveform for the sample light 40, with a single light pulse L for each ultrasound pulse U, the relative frequency shift between the sample light 40 and the reference light 42 and the pulse width of the sample light 40 can be selected in accordance with the equation:

f_{shift}*Tₒₚ=1, [31]

wherein f_{shift} equals frequency at which the sample light 40 is shifted relative to the reference light 42 by the interferometer in Hertz, and Tₒₚ is the pulse width of the sample light 40 in seconds. In this manner, as will be described in further detail below, any contribution of the time varying component of the background light 46 to the interference light pattern 48 is eliminated from the measurement. Thus, if the pulse width of the sample light 40 is 200 ns, the relative frequency shift f_{shift} between the sample light 40 and the reference light 42 should be 5 MHz. As a result, the frequency differences between the ultrasound tagged signal light 44 and the reference light 42 will be f_{shift}+fᵤₛ=−5 MHz+1 MHz=−4 MHz (1^{st} order), f_{shift}−fᵤₛ=−5 MHz−1 MHz=−6 MHz (1^{st} order), f_{shift}+2fᵤₛ=−5 MHz+2 MHz=−3 MHz (2^{nd} order), f_{shift}−2fᵤₛ=−5 MHz−2 MHz=−7 MHz (2^{nd} order), etc.; and the frequency difference between the untagged background light 46 and the reference light 42 will be 0 MHz.

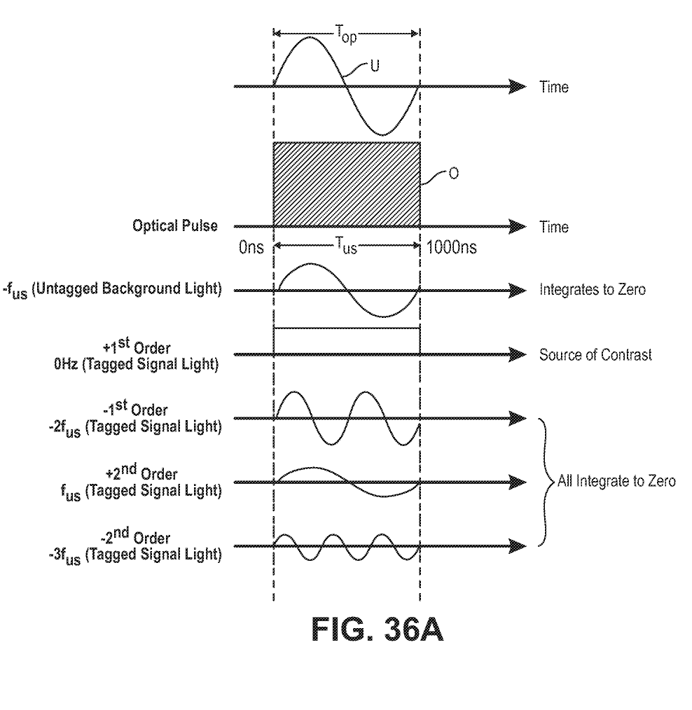

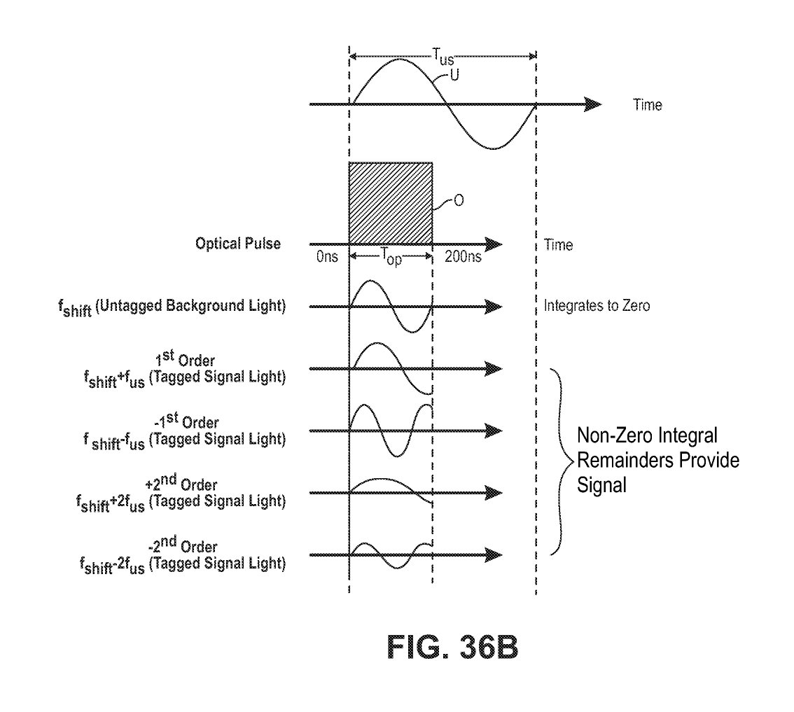

As one example, and with reference to FIG. 36a, it can be seen that when the duration Tₒₚ of the single light pulse L is matched to the duration Tᵤₛ (in this case, the wavelength) of the ultrasound pulse U, and the frequency difference between the sample light 40 and the reference light 42 is equal to the ultrasound frequency fᵤₛ (e.g., 1 MHz), the source of the contrast is a DC component resulting from the homodyne combination of the signal light 44 (tagged) and the reference light 42 at the first order (i.e., frequency difference between the signal light 44 and the reference light 42 is 0 Hz), which integrates to a non-zero value over the duration of the light pulse L (see equations [18] and [19] or equations [22]-[25]).

As can be readily seen in FIG. 36a, assuming that the sample light 40 has been downshifted by the ultrasound frequency fᵤₛ, the +1^{st} order heterodyne combination of the background light 46 and the reference light 42 (i.e., the frequency difference between the background light 46 (untagged) and the reference light 42 is −fᵤₛ) integrates to zero, thereby advantageously eliminating any contribution of the background light 46 to the interference light pattern 48 from the measurement. Coincidentally, the −1^{st} order heterodyne combination of the signal light 44 (tagged) and the reference light 42 (i.e., the frequency difference between the signal light 44 and the reference light 42 is −2fᵤₛ (e.g., −2 MHz)); the ±2^{nd} order heterodyne combination of the signal light 44 (tagged) and the reference light 42 (i.e., the frequency difference between the signal light 44 and the reference light 42 is +fᵤₛ (e.g., +1 MHz)); and the −2^{nd} order heterodyne combination of the signal light 44 (tagged) and the reference light 42 (i.e., the frequency difference between the signal light 44 and the reference light 42 is −3fᵤₛ (e.g., −3 MHz)) also integrates to zero, thereby eliminating any contribution of the signal light 44 to the interference light pattern 48 from the measurement as well. Notably, higher orders of heterodyne combinations are possible, but are not shown in FIG. 36a for purposes of brevity.

Thus, only the homodyne combination of the ultrasound tagged signal light 44 and the reference light 42 contributes to the interference light pattern 48 during measurement in this case.

In contrast, and with reference now to FIG. 36b, it can be seen that when the duration Tₒₚ of the single light pulse L (i.e., the pulse width of the sample light 40) is less than the duration Tᵤₛ (in this case, the wavelength) of the ultrasound pulse U, but the frequency difference f_{shift}(e.g., −5 MHz) between the sample light 40 and the reference light 42 is different from the ultrasound frequency fᵤₛ (e.g., 1 MHz), the source of the contrast are AC components resulting from the heterodyne combination of the ultrasound tagged signal light 44 and the reference light 42 at the +1^{st} order (i.e., frequency difference between the signal light 44 and the reference light 42 is f_{shift}+fᵤₛ (e.g., −4 MHz)); at the −1^{st} order (i.e., frequency difference between the signal light 44 and the reference light 42 is f_{shift}−fₕₛ (e.g., −6 MHz)), at the +2^{nd} order (i.e., frequency difference between the signal light 44 and the reference light 42 is f_{shift}+2fᵤₛ (e.g., −3 MHz)), and at the −2^{nd} order (i.e., frequency difference between the signal light 44 and the reference light 42 is f_{shift}−2fᵤₛ)(e.g., −7 MHz)), which all integrate to non-zero values over the duration of the light pulse L, as will be described in further detail below. Notably, higher orders of heterodyne combinations are possible, but are not shown in FIG. 36b for purposes of brevity.

Furthermore, when the frequency f_{shift} between the sample light 40 and the reference light 42 and the duration Tₒₚ of the single light pulse L are selected in accordance with equation [31] (and in the exemplary case, the frequency shift f_{shift} is selected to be 5 MHz), and the light pulse duration Tₒₚ is selected to be 200 ns), the heterodyne combination of the background light 46 and the reference light 42 (i.e., the frequency difference between the background light 46 and the reference light 42 is −f_{shift} (e.g., 5 MHz)) integrates to zero, thereby eliminating any contribution of the background light 46 to the interference light pattern 48 from the measurement.

It should be appreciated that, although the selection of the frequency f_{shift} between the sample light 40 and the reference light 42 and the light pulse duration Tₒₚ in accordance with equation [31] has been described in the context of a light pulse L having a shorter duration Tₒₚ than the duration Tᵤₛ of the ultrasound pulse U, in order to eliminate any contribution of the background light 46 to the interference light pattern 48 from the measurement, the frequency f_{shift} between the sample light 40 and the reference light 42 and the duration Tₒₚ of the light pulse L can be selected in accordance with equation [31] in the context of any relative duration between the light pulse L (or combined duration dₒₚ of light pulse(s) L) and the ultrasound pulse U, including if the combined duration dₒₚ of light pulse(s) L is greater than the duration Tᵤₛ of the ultrasound pulse U, with the accompanying advantage of eliminating any contribution of the background light 46 to the interference light pattern 48 from the measurement.

Referring back to FIG. 34, the interferometer 522 of the UOT system 510 concurrently generates an M number of interference patterns 48 via phase modulation, the spatial component values of which are concurrently detected over the M number of phases via the M number of optically registered detector arrays 228 (i.e., cameras or distinct camera regions) in the same manner described above with respect to FIG. 15. The UOT system 510 may, e.g., utilize any of the optical detection assemblies 200 illustrated in FIGS. 18-28 for performing these functions.

Once the detector arrays 228 acquire the data voxel by storing all spatial component values of the interference light patterns 48, these data can be sent to the processor 30, which, as discussed above, is configured for determining a physiologically-dependent optical parameter (e.g., absorption) of the target voxel 14 based on the acquired spatial component values of the interference light patterns 48. As briefly discussed above, the spatial component values can be power values, which can be used by the processor 30 to reconstruct the amplitude of the signal light 44, and thus, can be said to be representative of the physiologically-dependent optical parameters (e.g., optical absorption) of the target voxel 14.

In a UOT system 510 having two cameras or camera regions, such as illustrated in the optical detection assemblies 200a of FIGS. 18-21, the interference terms will be 180 degrees out of phase for the two cameras or camera regions 228a, 228b. The processor 30 may reconstruct the amplitude of the signal light 44 in accordance with the following equations. Assuming that the sample light 40 has a single rectangular pulse over the relevant time period of 1/f_{shift}, and that the interference term between the signal light 44 and the background light 46 oscillate at the ultrasound frequency fᵤₛ, given appropriate light pulse duration and integration time, and thus cannot be detected by the cameras 228a, 228b, the power detected at a kth pixel for the first camera 228a can be expressed as:

value_{1,k}=∫₀^{T}^{ₒₚ}(P_{background}+P_{signal}+P_{reference}+2\sqrt{P_{signal}×P_{reference}}×(sin(2π(−f_{shift}+fᵤₛ)t+θ_{unknown,speckle k})+sin(2π(−f_{shift}−fᵤₛ)t+Θ_{unknown,speckle k})+sin(2π(−_{shift}+2fᵤₛ)t+ϕ_{unknown,speckle k})+sin(2π(−f_{shift}−2fᵤₛ)t+Φ_{unknown,speckle k}))+2\sqrt{P_{background}×P_{reference}}×sin(−2πf_{shift}t+α_{unknown,speckle k}))dt, [32]

and likewise, the power detected at the corresponding kth pixel of the second camera 228b can be expressed as:

Value₂ₖ=∫₀^{T}^{ₒₚ}(P_{background}+P_{signal}+P_{reference}+2\sqrt{P_{signal}×P_{reference}}×(sin(2π(−f_{shift}+fᵤₛ)t+θ_{unknown,speckle k}+π)+sin(2π(−f_{shift}−fᵤₛ)t+Θ_{unknown,speckle k}+π)+sin(2π(−f_{shift}+2fᵤₛ)t+ϕ_{unknown,speckle k}+π)+sin(2π(−f_{shift}−2fᵤₛ)t+Φ_{unknown,speckle k}+π))+2\sqrt{P_{background}×P_{reference}}×sin(−2πf_{shift}t+α_{unknown,speckle k}+π))dt. [33]

where P background represents light at frequency f−f_{shift} that has not been tagged with the ultrasound 32; P_{signal} represents light at frequency f−f_{shift} (fᵤₛ, −fᵤₛ, 2fᵤₛ, −2fᵤₛ) that has been tagged with the ultrasound 32 at multiple orders; P_{reference} represents the reference light at frequency f; Tₒₚ is the duration of a single rectangular pulse of the sample light 40; and θ_{unknown, speckle k}, Θ_{unknown, speckle k}, ϕ_{unknown, speckle k}, Φ_{unknown, speckle k}, and α_{unknown, speckle k} are random phases at the kth speckle grain at the time of measurement, which originate via multiple scattering of coherent light inside the tissue.

Over the duration of the light pulse L (Tₒₚ), equations [32] and [33] respectively integrate to:

Value_{,k}=(P_{background}+P_{signal}+P_{reference})Tₒₚ−2\sqrt{P_{signal}×P_{reference}}×[(cos((2πf_{shift}+fᵤₛ))Tₒₚ+θ_{unknown,speckle k})−cos(θ_{unknown,speckle k}))/(2π−(f_{shift}+fᵤₛ))+(cos((2π(−f_{shift}−fᵤₛ))TₒₚΘ_{unknown,speckle k})−cos(Θ_{unknown,speckle k}))/(2π−(f_{shift}−fᵤₛ))+(cos((2πf_{shift}+2fᵤₛ))Tₒₚ+ϕ_{unknown,speckle k})−cos(ϕ_{unknown,speckle k}))/(2π(−f_{shift}+2fᵤₛ)+(cos((2π(−f_{shift}+2fᵤₛ))Tₒₚ+Φ_{unknown,speckle k})−cos(Φ_{unknown,speckle k}))/(2π(−f_{shift2}−fᵤₛ)]+2\sqrt{P_{background}×P_{reference}}×(cos((−2πf_{shift})Tₒₚ+α_{unknown,speckle k})−cos(α_{unknown,speckle k}))/2πf_{shift}) [34]

Significantly, because the product of the between frequency difference between the background light 46 and the reference light 42 (f_{shift}) and the duration of the light pulse L (Tₒₚ) equals 1 in accordance with equation [31], the interference term between the reference light 42 and the background light 46 integrates to zero. Furthermore, the terms P_{background}+P_{signal}+P_{reference} are constant across the two cameras 228a, 228b, and thus, can be eliminated by taking the difference of the intensity values Valueₖ between the two cameras 228a, 228b, leaving only the difference of the interference terms between the reference light 42 and the signal light 44, as follows for the two cameras 228a, 228b:

Value_{1,k} Value_{2,k}=−2\sqrt{P_{signal}×P_{reference}}×[(cos(2π(−f_{shift}+fᵤₛ)Tₒₚ+θ_{unknown,speckle k})−cos(θ_{unknown,speckle k}(2π(−f_{shift}+fᵤₛ))+(cos(2π(−f_{shift}−fᵤₛ)Tₒₚ+Θ_{unknown,speckle k})−cos(Θ_{unknown,speckle k}))/(2π(−f_{shift}−fᵤₛ))+(cos(2π(−f_{shift}+2fᵤₛ)Tₒₚ+Ø_{unknown,speckle k})−cos(Ø_{unknown,speckle k}))/(2π(f_{shift}−2fᵤₛ))+(cos(2π(−f_{shift}−2fᵤₛ)Tₒₚ+Φ_{unknown,speckle k})−cos(Φ_{unknown,speckle k}))/(2π(−f_{shift}+2fᵤₛ))]+2\sqrt{P_{signal}×P_{reference}}×[(cos(2π(−f_{shift}+fᵤₛ)Tₒₚ+θ_{unknown,speckle k}+π)−cos(θ_{unknown,speckle k}+π))/(2π(−f_{shift}+fᵤₛ))+(cos(2π(−f_{shift}+fᵤₛ)Tₒₚ+Θ_{unknown,speckle k π})−cos(Θ_{unknown,speckle k}))/(2π(−f_{shift}−fᵤₛ))+(cos(2π(−f_{shift}+2fᵤₛ)Tₒₚ+Ø_{unknown,speckle k}+π)−cos(Ø_{unknown,speckle k}+π))/(2π(−f_{shift}+2fᵤₛ))+(cos(2π(−f_{shift}−2fᵤₛ)Tₒₚ+Φ_{unknown,speckle k}+π)−cos(Φ_{unknown,speckle k}+π))/(2π(−f_{shift}+2fᵤₛ))] [35]

which can be summed over all of the k pixels and then averaged in accordance with equation [19] set forth above. Based on known scaling relationship between the power of the reference light P_{reference} and the power of the signal light P_{signal} discussed above, the power of the P_{signal} can be expressed in accordance with equations [20] or [21] set forth above.

Thus, it can be appreciated that the intention of the two-bin measurement is to arrive at a precise estimate of power of the signal light P_{signal} up to the aforementioned scaling relationship defined by the strength of the reference light P_{reference}, by removing the terms that are constant between the two measurements of the cameras 228a, 228b, removing the unknown speckle-specific phases, and extracting only the amplitude of the cosine term. As with the two-bin detection scheme described above with respect to the UOT system 10, with an N number of pixels, the signal-to-noise ratio (SNR) scales with N^{1/2} in accordance with equation [13] set forth above.

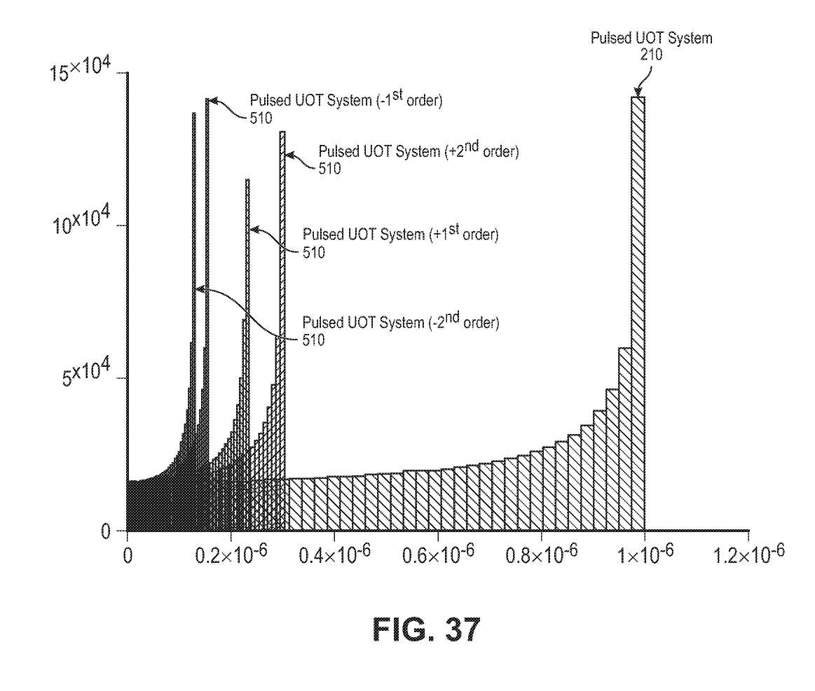

Assuming a rectangular pulsed waveform for the sample light 40, and a frequency shift f_{shift} of −5 MHz, an ultrasound frequency fᵤₛ of 1 MHz (equivalent to an ultrasound pulse duration Tᵤₛ of 1000 ns), and a light pulse duration Tₒₚ of 200 ns, a 1^{st} order ultrasound tagging efficiency of 5%, and a 2^{nd} order ultrasound tagging frequency of 3%, it has been shown that the UOT system 510 of FIG. 34 provides lower, but sufficient signal. For example, as illustrated in FIG. 37, a histogram of the population of energy for the UOT system 210 that uses a light pulse duration Tₒₚ of 1000 ns and an ultrasound frequency fᵤₛ of 1 MHz, as compared to the population of energy for the ±1^{st} order and ±2^{nd} order Raman-Nath components of the UOT system 510 that uses a light pulse duration Tₒₚ of 200 ns, an ultrasound frequency fᵤₛ of 1 MHz, and a frequency shift f_{shift} between the sample light 40 and the reference light 42 of 5 MHz, reveals that relative strengths of the +1^{st} order, −1^{st} order, +2^{nd} order, and −2^{nd} order Raman-Nath components are 23%, 16%, 30%, and 13%.

It should be appreciated that although the sample light 40 has been described as having a pulsed waveform shape with a single rectangular pulse for each cycle of the frequency shift f_{shift} between the sample light 40 and the reference light 42, as illustrated in FIGS. 32a and 32b, the sample light 40 may have other pulsed waveform shapes, including a pulsed waveform shape with double pulses for each cycle of the frequency shift f_{shift} between the sample light 40 and the reference light 42. In these cases, different functions between the frequency shift f_{shift} between the sample light 40 and the reference light 42 and the waveform of the sample light 40 other than that discussed above will need to be used to eliminate the contribution of background light 46 to the interference light pattern 47. As a general rule, a function of the shape of the pulsed waveforms for the sample light 40 and the frequency shift f_{shift} between the sample light 40 and the reference light 42 can be selected in accordance with the following equation, such that any contribution of time varying component of the background light 46 to the interference light pattern 47 is eliminated:

$${\int }_{0}^{1 / {f}_{shift}} \sqrt{{P}_{reference} ( t ) * {P}_{background} ( t )} \times sin ( 2 π {f}_{shift} + {α}_{unknown} ) dt \approx 0 , \qquad [ 36 ]$$

where P_{reference}(t) is the time varying component of the reference light 42 as a function of time t, P_{background}(t) is the time varying component of the background light 46 as a function of time t, and f_{shift} equals the frequency at which the sample light 40 is shifted relative to the reference light 42 by the interferometer 522, and α_{unknown} is an unknown phase.

It should be appreciated that it is preferred that equation [36] hold true for all possible values of the unknown phase α_{unknown}. If the sample light 40 has a single rectangular pulse per ultrasound pulse (i.e., the pulse is constant), the DC product of the reference light 42 and background light 46 can be removed from the integral, and equation [36] reduces to:

\sqrt{P_{reference}(t)*P_{background}(t)}×∫₀^{T}^{ₒₚ}sin(2πf_{shift}+a_{unknown})dt= [³⁷]

Over the duration of the light pulse L (Tₒₚ), equation [37] integrates to:

−\sqrt{P_{reference}(t)*P_{background}(t)}/2πf_{shift}*(cos(2πf_{shift}*Tₒₚ+α_{unknown})−cos(α_{unknown})) [38]

If the frequency shift f_{shift} and the optical pulse duration Tₒₚ are selected in accordance with equation [31], equation [38] will be satisfied for every value of phase α_{unknown}, and the interference product between the reference light 42 and the background light 46 integrates to zero.

If the pulses of the sample light 40 vary over time, it has been discovered that if the sample light 40 has a pulsed waveform shape with double pulses for each cycle of the frequency shift f_{shift} between the sample light 40 and the reference light 42, for any selection of a pair of identically shaped pulses for the sample light 40, equation [38] will be satisfied for every value of phase α_{unknewn} (and the interference product between the reference light 42 and the background light 46 integrates to zero) if the identically shaped pulses are separated from each other by:

dₛₑₚₐᵣₐₜᵢₒₙ=½*f_{shift}, [39]

where dₛₑₚₐᵣₐₜᵢₒₙ is the separation between any point on the first pulse and the corresponding point on the second pulse.

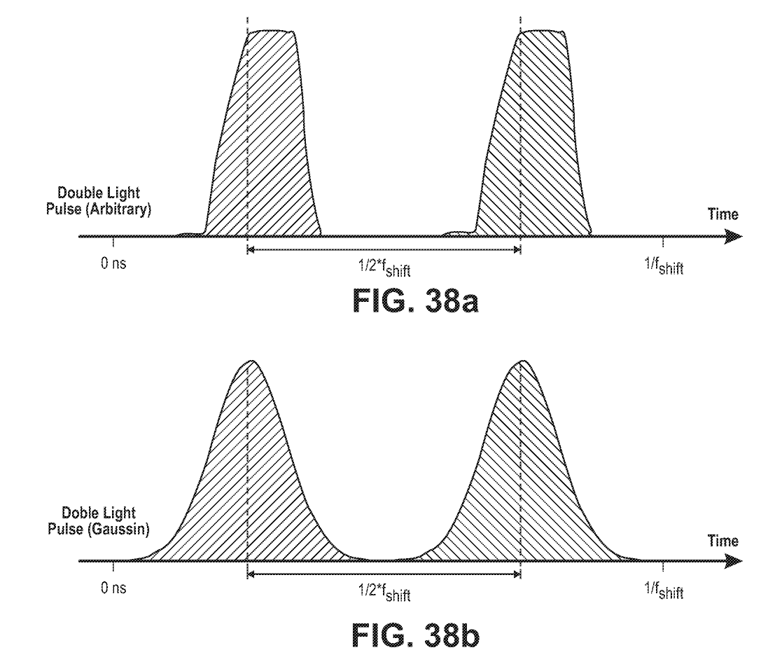

As one example illustrated in FIG. 38a, two identical arbitrarily-shaped pulses L will satisfy equation [36] when the separation dₛₑₚₐᵣₐₜᵢₒₙ between two corresponding points on the pulses L is equal to ½*f_{shift}. As illustrated in FIG. 38b, it follows that two identical symmetrical pulses, and in this case, Gaussian pulses, will satisfy equation [36] when the separation dₛₑₚₐᵣₐₜᵢₒₙ between two corresponding points on the pulses L is equal to ½*f_{shift}.

It should be appreciated that, although it may be optimal to completely eliminate all contribution of background light 46 to the interference light pattern 48, it is still desirable to eliminate approximately all of the contribution of the background light 46 to the interference light pattern 48 (i.e., the interference product between the background light 46 and the reference light 46 in equation [36] approximately equals 0). For the purposes of this specification, the interference product between the background light 46 and the reference light 46 in equation [36] approximately equals 0 if at least 90 percent of the time varying component of P_{background}(t) has been eliminated from the interference light pattern 48. Preferably, at least 99 percent of the time varying component of P_{background}(t) has been eliminated from the interference light pattern 48.

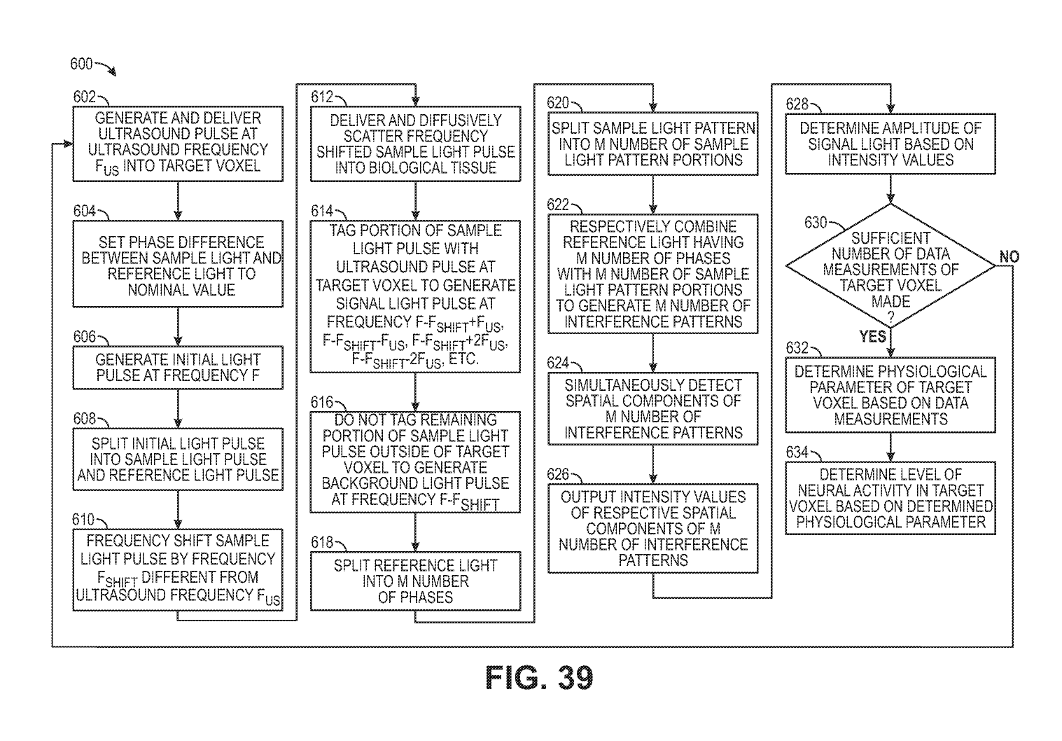

Referring to FIG. 39, one particular method 600 performed by the UOT system 510 to non-invasively measure the target voxel 14 in an anatomical structure 16 will now be described. Prior to operating the UOT system 510, the wavelength (and thus, the frequency f) of the sample light 40 may be selected based on the physiologically-dependent optical parameter to be ultimately determined; the ultrasound frequency fᵤₛ may be selected to maximize the pressure at the target voxel 14, the pulse width of the ultrasound 32 may be selected based on the ultrasound frequency fᵤₛ (e.g., such that there is one ultrasound cycle per pulse), the combined duration dₒₚ of the pulse(s) of the sample light 40 (pulse duration tₒₚ in the case of a single pulse) is selected to be less than the pulse width of the ultrasound 32 (e.g., such that an off-the-shelf laser source 50 may be used), and the frequency shift f_{shift} between the sample light 40 and the reference light 42 and pulsed waveform of the sample light 40 are selected in accordance with equation [36]).

After the design parameters of the UOT system 510 are set, in much the same way that the UOT system 210 performs steps 402-408 with respect to FIG. 31, the controller 24 of the UOT system 510 operates the acoustic assembly 20 to generate and deliver a pulse of ultrasound 32 having a frequency fᵤₛ into the anatomical structure 16 (step 602), sets the phase difference between the sample light 40 and the reference light 42 to a nominal value (e.g., 0) (step 604), and operates the interferometer 522 to generate and emit a pulse of source light 38 having a frequency f (step 606); and the interferometer 522 (e.g., via the beam splitter 52) splits the pulse of source light 38 into a pulse of sample light 40 and a pulse of reference light 42 (step 608).

Next, prior to the pulse of sample light 40 entering the anatomical structure 16, the controller 24 operates the interferometer 522 to frequency shift the pulse of sample light 40 by a frequency f_{shift} that is different from the ultrasound frequency fᵤₛ (as opposed to shifting the pulse of sample light 40 by the ultrasound frequency fᵤₛ as is performed in step 410 of FIG. 31), resulting in the pulse of sample light 40 having a frequency f−f_{shift} (step 610). It should be appreciated that, although this frequency shifting technique implements the frequency shifting technique illustrated in FIG. 35a, other frequency shifting techniques can be utilized, such as those illustrated in FIGS. 33b-35d.

The frequency-shifted pulse of sample light 40 is then delivered into and diffusively scattered within the anatomical structure 16 (step 612). As the pulse of frequency shifted sample light 40 scatters diffusively through the anatomical structure 16, a portion will pass through the target voxel 14 and be frequency shifted (i.e., tagged) by the pulse of ultrasound 32 passing through the target voxel 14 to the frequencies f−f_{shift}+fᵤₛ (1^{st} order), f−f_{shift}−fᵤₛ (1^{st} order), f−f_{shift}+2fᵤₛ (2^{nd} order), f−f_{shift}−2fᵤₛ (2^{nd} order), etc., resulting in a pulse of scattered signal light 44 having the same frequency f (step 614); and the remaining portion will not pass through the target voxel 14, and thus will not be frequency shifted by the pulse of ultrasound 32, resulting in a pulse of scattered background light 46 having a frequency f−f_{shift} (the same frequency as the frequency shifted sample light 40 prior to entering the anatomical structure 16) (step 616).

Next, in much the same way that the UOT system 210 performs steps 418-426 with respect to FIG. 31, the interferometer 522 then concurrently, via the one or more beam splitter/combiners 258, splits the reference light 42 respectively into an M number of different phases (step 618), splits the sample light pattern 47 into an M number of sample light pattern portions (step 620), and respectively combines the M number of sample light pattern portions and the reference light 42 to respectively generate the M number of interference light patterns 48 (step 622); and under control of the controller 24, each of M number of detector arrays 228 simultaneously detect respective spatial components of the corresponding interference light pattern 48 (i.e., speckle grains in the case where the interference light pattern includes a speckle light pattern) (step 624), and output intensity values for the respective spatial components of the N number of interference light patterns 48 (step 626). The processor (e.g., a portion of which may include processing circuitry 306) then determines the amplitude of the signal light 44 based on these intensity values (e.g., using equation [35] and equations [20] or [21] in the case where M equal two) (step 628).

In much the same way that the UOT system 210 performs steps 430-434 with respect to FIG. 31, steps 602-628 can be iterated to repeatedly acquire data measurements of the target voxel 14, and if a sufficient number of data measurements have been acquired (step 630), the processor 30 may then determine the physiologically-dependent optical parameter (e.g., level of deoxygenated and/or oxygenated hemoglobin concentration or relative abundance or level of water concentration or relative water concentration) of the target voxel 14 based on the data measurements (step 632); and in the case where the target voxel 14 is brain matter, the processor 30 may further determine the level of neural activity within the target voxel 14 based on the determined physiologically-dependent optical parameter (step 634), e.g., in the manner described above with respect to step 134 of the method 100 illustrated in FIG. 13.

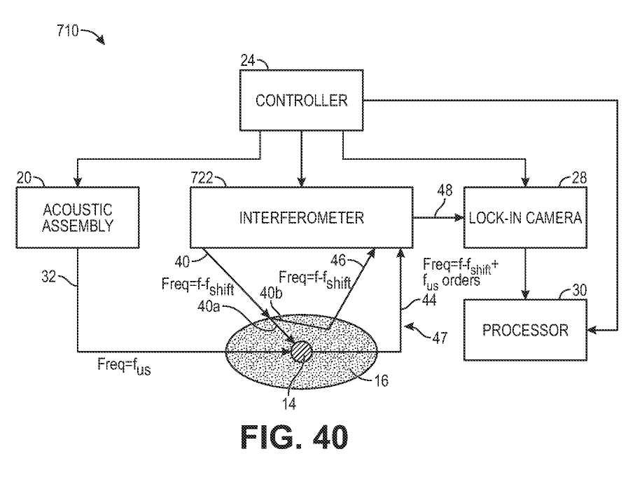

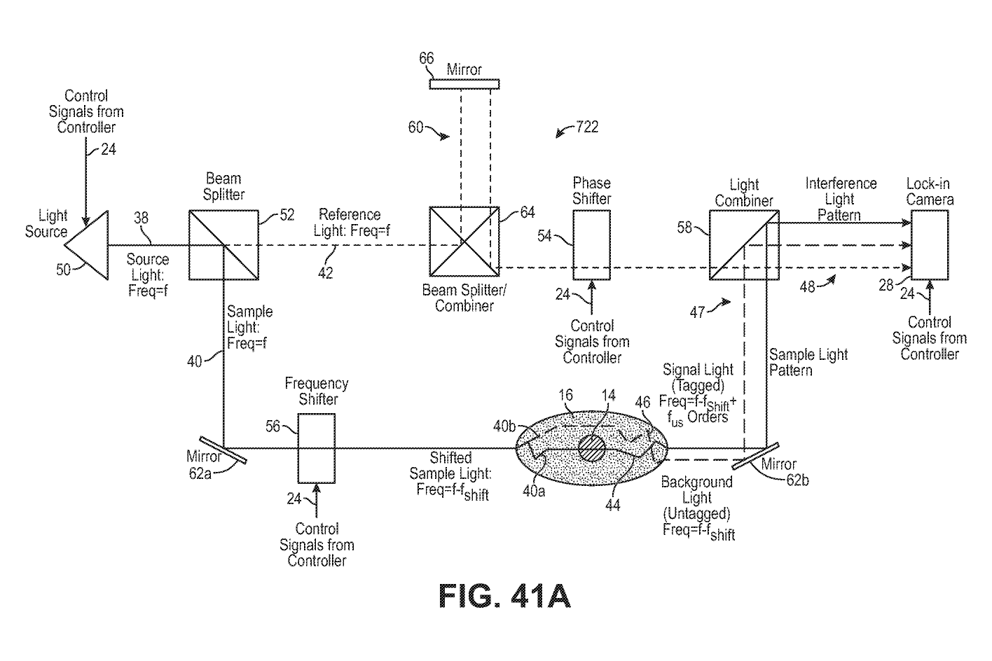

It should be noted that the heterodyne technique exemplified in FIG. 36b, and variations thereof, can be utilized in other types of UOT systems to maximize the signal detection efficiency when a shorter optical pulse width is used. As one example, and with reference to FIG. 40, another embodiment of a UOT system 710 is designed to combine the reference light 42 and signal light 44 in a heterodyne manner. In particular, the UOT system 710 is similar to the UOT system of FIG. 1, with the exception that UOT system 710 comprises an interferometer 722 that, much like the interferometer 522 described above, is configured for using sample light 40 having a relatively short pulse width, and shifting the frequency f of the sample light 40 and reference light 42 relative to each other by a frequency f_{shift} different from (rather than equal to) the frequency fᵤₛ of the ultrasound 32, such that, upon combining by the interferometer 722, the frequency of the ultrasound tagged signal light 44 will be shifted to a different frequency as the reference light 42, and the frequency of the signal light 44 differs from the frequency of the reference light 42 by f_{shift} (+1^{st} order) f_{shift}−fᵤₛ (−1^{st} order), f_{shift}+2fᵤₛ (+2^{nd} order), f_{shift}−2fᵤₛ (−2^{nd} order), etc.

The duration of the duration Tₒₚ of the sample light pulse L may be selected to be less than the duration Tᵤₛ of the ultrasound pulse U, as shown in FIG. 32a, with the associated advantages of minimizing the complexity and cost of the pulsed laser source, as well as improving the axial resolution, as described in detail above. Furthermore, the relative frequency shift f_{shift} between the sample light 40 and the reference light 42, and the pulse width of the sample light 40, may be selected in accordance with equation [31], such that any contribution of the background light 46 to the interference light pattern 48 is eliminated from the measurement.

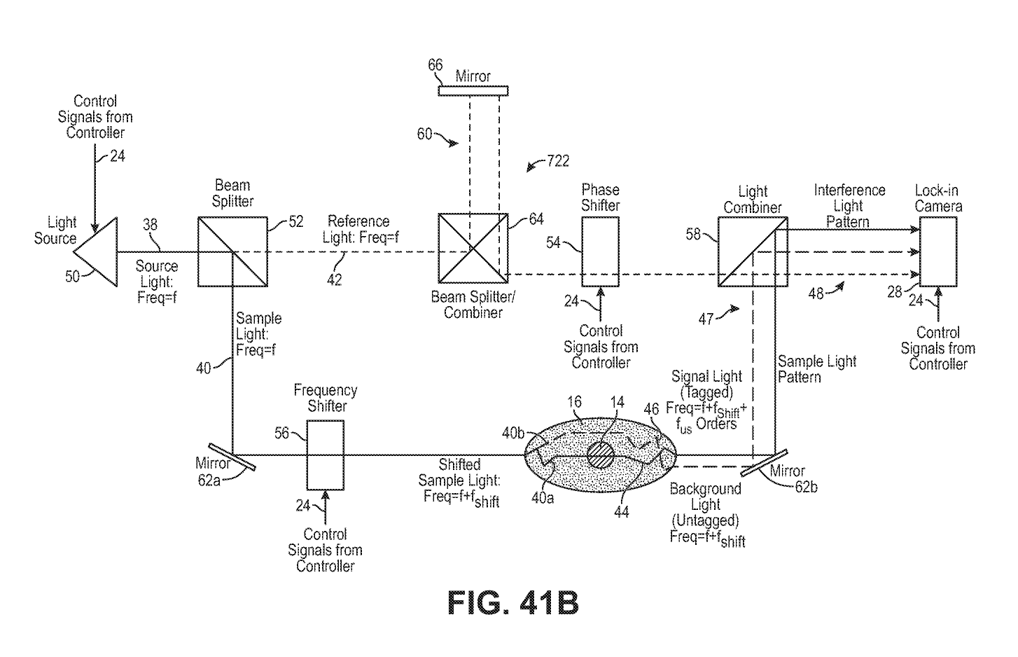

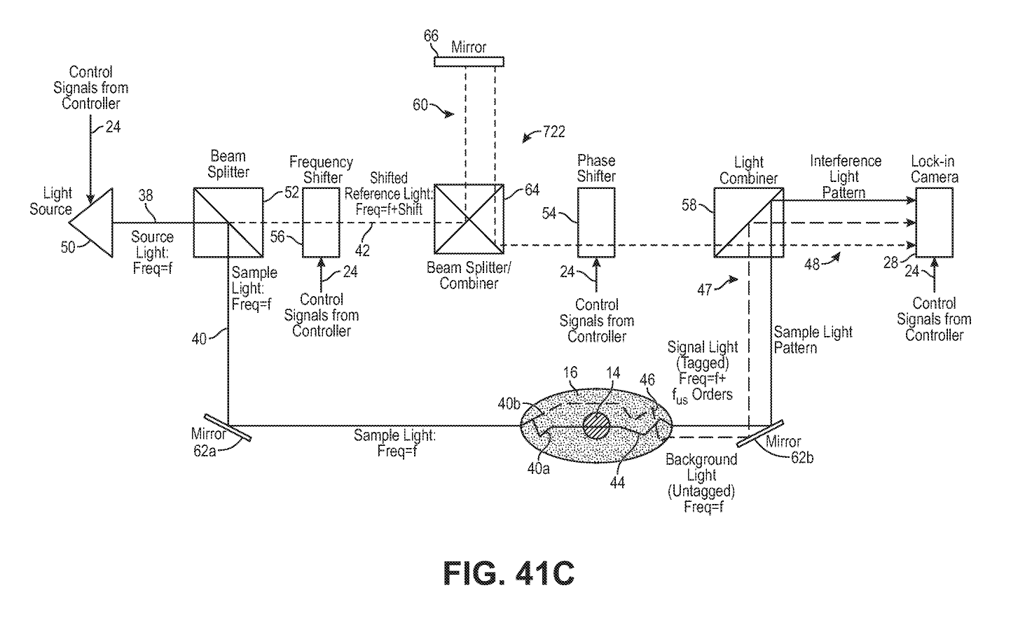

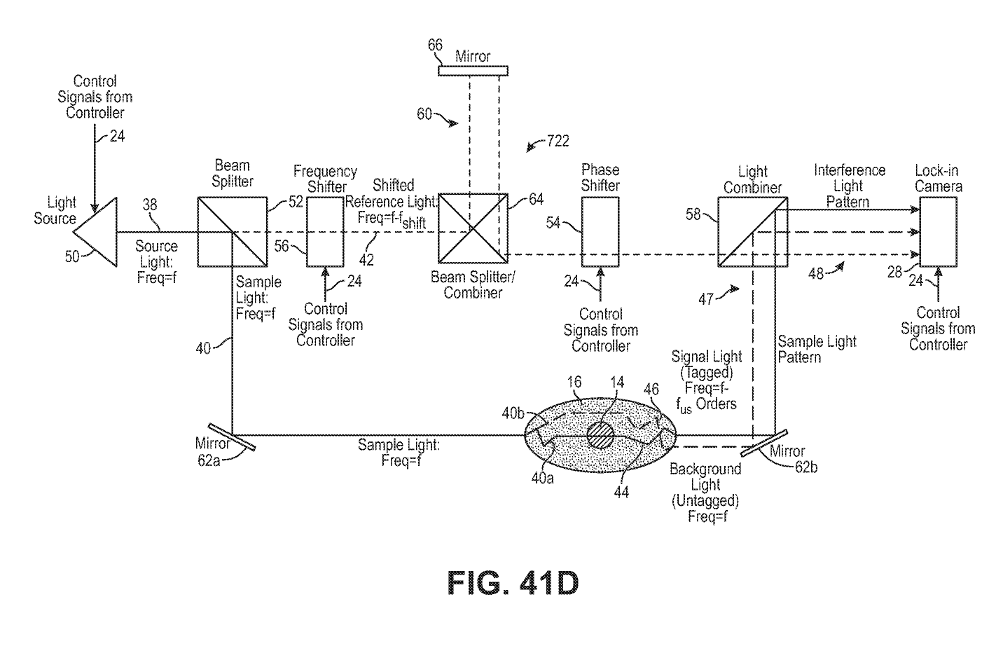

In the embodiment illustrated in FIG. 40, the interferometer 722 down frequency shifts the sample light 40 by the frequency f_{shift}, such that the ultrasound tagged signal light 44 has the frequencies f−f_{shift}+fᵤₛ (1^{st} order), f−f_{shift}−fᵤₛ (1^{st} order), f−f_{shift}+2fᵤₛ (2^{nd} order), f−f_{shift}−2fᵤₛ (2^{nd} order), etc.; the untagged background light 46 has the frequency f−f_{shift}; and the reference light 42 has a frequency f, thereby enabling combination of the reference light 42 and signal light 44 in a heterodyne manner, as better illustrated in interferometer 222 of FIG. 41a. However, the interferometer 722 may up frequency shift the sample light 40 by the frequency f_{shift}, such that, the ultrasound tagged signal light 44 has the frequencies f+f_{shift}+fᵤₛ (1^{st} order) f+f_{shift}−fᵤₛ (1^{st} order), f+f_{shift}+2fᵤₛ (2^{nd} order), f+f_{shift}−2fᵤₛ (2^{nd} order), etc., the untagged background light 46 has the frequency f+f_{shift}, and the reference light 42 has a frequency f (see FIG. 41b); may up frequency shift the reference light 42 by the frequency f_{shift}, such that the ultrasound tagged signal light 44 has the frequencies f+fᵤₛ (1^{st} order), f−fᵤₛ (1^{st} order), f+2fᵤₛ (2^{nd} order), f−2fᵤₛ (2^{nd} order), etc., the untagged background light 46 has the frequency f, and the reference light 42 has a frequency f+f_{shift} (see FIG. 41c); may down frequency shift the reference light 42 by the frequency f_{shift}, such that the ultrasound tagged signal light 44 has the frequencies f+fᵤₛ (1^{st} order), f−fᵤₛ (1^{st} order), f+2fᵤₛ (2^{nd} order), f−2fᵤₛ (2^{nd} order), etc., the untagged background light 46 has the frequency f, and the reference light 42 has a frequency f−f_{shift} (see FIG. 41d); or perform any other frequency shift of the sample light 40 or reference light 42 that results in the heterodyne combination of the reference light 42 and the signal light 44.

The UOT system 710, like the UOT system of FIG. 1, comprises a lock-in camera 28 with multiple data storing bins for respectively storing spatial component values used to extract the signal light 44 from the interference light pattern 48 in accordance with equations [3] or [6]-[10]. Once the lock-in camera 28 acquires the data voxel by storing all spatial component values of the interference light pattern 48, these data can be sent to the processor 30, which, as discussed above, is configured for determining a physiologically-dependent optical parameter (e.g., absorption) of the target voxel 14 based on the acquired spatial component values of the interference light pattern 48. As briefly discussed above, the spatial component values can be power values, which can be used by the processor 30 to reconstruct the amplitude of the signal light 44, and thus, can be said to be representative of the physiologically-dependent optical parameters (e.g., optical absorption) of the target voxel 14. The processor 30 may extract the amplitude of the signal light 44 in accordance with an equation similar to the equation [35] (with the exception that, instead of using two detector arrays (i.e., cameras or camera regions) a single detector array with two data storing bins are used) and equations [19]-[21].

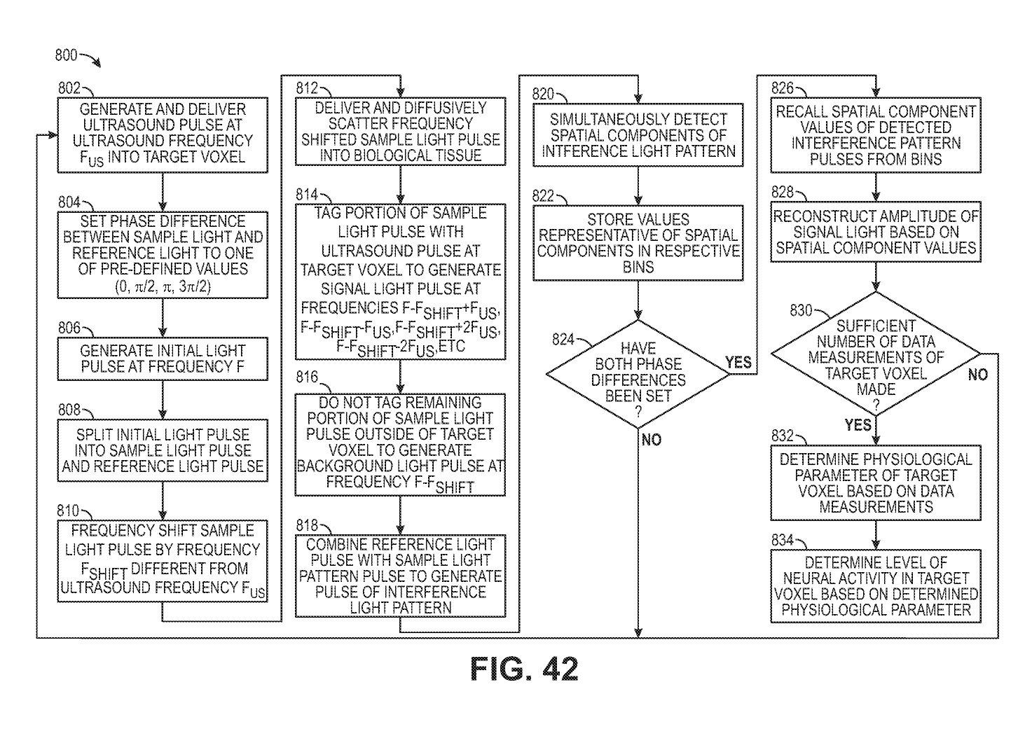

Referring to FIG. 42, one particular method 800 performed by the UOT system 710 to non-invasively measure the target voxel 14 in an anatomical structure 16 will now be described. Prior to operating the UOT system 710, the wavelength (and thus, the frequency f) of the sample light 40 may be selected based on the physiologically-dependent optical parameter to be ultimately determined; the ultrasound frequency fᵤₛ may be selected to maximize the pressure at the target voxel 14, the pulse width of the ultrasound 32 may be selected based on the ultrasound frequency fᵤₛ (e.g., such that there is one ultrasound cycle per pulse), the combined duration dₒₚ of the pulse(s) of the sample light 40 (pulse duration tₒₚ in the case of a single pulse) is selected to be less than the pulse width of the ultrasound 32 (e.g., such that an off-the-shelf laser source 50 may be used), and the frequency shift f_{shift} between the sample light 40 and the reference light 42 and pulsed waveform of the sample light 40 are selected in accordance with equation [36]).

After the design parameters of the UOT system 710 are set, in much the same way that the UOT system 210 performs steps 102-110 with respect to FIG. 13, the controller 24 of the UOT system 510 operates the acoustic assembly 20 to generate and deliver a pulse of ultrasound 32 having a frequency fᵤₛ into the anatomical structure 16 (step 802), sets the phase difference between the sample light 40 and the reference light 42 to one of the two pre-defined values (0 and π) (step 804), and operates the interferometer 522 to generate and emit a pulse of source light 38 having a frequency f (step 806); and the interferometer 522 (e.g., via the beam splitter 52) splits the pulse of source light 38 into a pulse of sample light 40 and a pulse of reference light 42 (step 808).

Next, prior to the pulse of sample light 40 entering the anatomical structure 16, the controller 24 operates the interferometer 522 to frequency shift the pulse of sample light 40 by a frequency f_{shift} that is different from the ultrasound frequency fᵤₛ (as opposed to shifting the pulse of sample light 40 by the ultrasound frequency fᵤₛ as is performed in step 210 of FIG. 13) (step 810). It should be appreciated that, although this frequency shifting technique implements the frequency shifting technique illustrated in FIG. 41a, other frequency shifting techniques can be utilized, such as those illustrated in FIGS. 41b-41d.

The frequency-shifted pulse of sample light 40 is then delivered into and diffusively scattered within the anatomical structure 16 (step 812). As the pulse of frequency shifted sample light 40 scatters diffusively through the anatomical structure 16, a portion will pass through the target voxel 14 and be frequency shifted (i.e., tagged) by the pulse of ultrasound 32 passing through the target voxel 14 to the frequencies f−f_{shift}+fᵤₛ (1^{st} order), f−f_{shift}−fᵤₛ (1^{st} order), f−f_{shift}+2fᵤₛ (2^{nd} order), f−f_{shift}+2fᵤₛ (2^{nd} order), etc., resulting in a pulse of scattered signal light 44 having the same frequency f (step 814); and the remaining portion will not pass through the target voxel 14, and thus will not be frequency shifted by the pulse of ultrasound 32, resulting in a pulse of scattered background light 46 having a frequency f−f_{shift} (the same frequency as the frequency shifted sample light 40 prior to entering the anatomical structure 16) (step 816).

Next, in much the same way that the UOT system 110 performs steps 418-426 with respect to FIG. 13, the interferometer 722 then combines the pulse of reference light 42 with the pulse of the sample light pattern 47 to generate a pulse of an interference light pattern 48 (step 818). Then, under control of the controller 24, all of the detectors 68 (FIG. 4) of the lock-in camera 28 simultaneously detect respective spatial components of the interference light pattern 48 (i.e., speckle grains in the case where the interference light pattern includes a speckle light pattern) (step 820), and values (e.g., power values) representative of the spatial components of the interference light pattern 48 are stored in data storing bins 70 (initially, the first bins 70a of the corresponding detectors 68) (step 822). If the interferometer 222 has not been set to both phases (step 824), the controller 24 then repeats steps 802-822 to take the last measurement.

After measurements have been taken, the controller 24 recalls the spatial component values of the detected interference light pattern pulses 48 from the data storing bins 70 of the lock-in camera 28 and transfers these values to the processor 30 (step 826). The processor 30 reconstructs the amplitude of the signal light 44 from the two interference light patterns 48 based on these spatial component values (e.g., by using an equation similar to the equation [35] (with the exception that, instead of using two detector arrays (i.e., cameras or camera regions) a single detector array with two bins are used) and equations [19]-[21].

In much the same way that the UOT system 110 performs steps 130-134 with respect to FIG. 13, steps 802-828 can be iterated to repeatedly acquire data measurements of the target voxel 14, and if a sufficient number of data measurements have been acquired (step 830), the processor 30 may then determine the physiologically-dependent optical parameter (e.g., level of deoxygenated and/or oxygenated hemoglobin concentration or relative abundance or level of water concentration or relative water concentration) of the target voxel 14 based on the data measurements (step 832); and in the case where the target voxel 14 is brain matter, the processor 30 may further determine the level of neural activity within the target voxel 14 based on the determined physiologically-dependent optical parameter (step 834), e.g., in the manner described above with respect to step 134 of the method 100 illustrated in FIG. 13.

It should be appreciated that any type of pulsed UOT system, e.g., an off-axis pulsed UOT holographic system, may benefit from the use of a shorter optical pulse width, with the associated advantages described herein. Furthermore, selecting the pulsed waveform of the sample light 40 and the frequency shift f_{shift} between the sample light 40 and the reference light 42 in accordance equation [36] in order to eliminate the contribution of the background light 46 to the interference pattern(s) 47, any implementation of a pulsed UOT system can be used to detect the remaining signal components in the interference pattern(s) 47.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A pulsed ultrasound modulated optical tomography (UOT) system, comprising:
an interferometer configured for delivering sample light into the anatomical structure having a target voxel, whereby a portion of the sample light passing through the target voxel is scattered by the anatomical structure as signal light, and another portion of the sample light not passing through the target voxel is scattered by the anatomical structure as background light that is combined with the signal light to create a sample light pattern, the interferometer is further configured for combining reference light with the sample light pattern to generate at least one interference light pattern, such that at least one component of the signal light and the reference light are combined in a heterodyne manner;
an acoustic assembly configured for delivering ultrasound into the target voxel, such that the signal light is frequency shifted by the ultrasound;
a controller configured for operating the acoustic assembly and the interferometer to pulse the ultrasound and the sample light in synchrony at the target voxel, such that at least one pulse of the sample light has a combined duration less than a pulse width of the ultrasound;
at least one array of detectors configured for detecting spatial components of each of the at least one interference light pattern and storing a plurality of values representative of the respective spatial components of each of the at least one interference light pattern; and
a processor configured for determining a physiologically-dependent optical parameter of the target voxel based on the plurality of values.

2. The pulsed UOT system of claim 1, wherein the at least one pulse of the sample light comprises only a single pulse.

3. The pulsed UOT system of claim 1, wherein the at least one pulse of the sample light comprises a plurality of pulses.

4. The pulsed UOT system of claim 1, wherein the interferometer is further configured for shifting the sample light relative to the reference light by a frequency different from the frequency of the ultrasound, such that the signal light and the reference light are combined in the heterodyne manner.

5. The pulsed UOT system of claim 4, wherein
$${\int }_{0}^{1 / {f}_{shift}} \sqrt{{P}_{reference} ( t ) * {P}_{background} ( t )} \times sin ( 2 π {f}_{shift} + {α}_{unknown} ) dt \approx 0 ,$$
wherein P_{reference}(t) is the time varying component of the reference light as a function of time t, P_{background}(t) is the time varying component of the background light as a function of time t, and f_{shift} equals the frequency at which the sample light is shifted relative to the reference light by the interferometer.

6. The pulsed UOT system of claim 5, wherein at least 99% of P_{background}(t) is eliminated from the interference light pattern.

7. The pulsed UOT system of claim 5, wherein the at least one pulse of the sample light is a single rectangular pulse, and wherein f_{shift}*Tₒₚ=1, wherein Tₒₚ is the width of the single rectangular pulse.

8. The pulsed UOT system of claim 5, wherein the at least one pulse of the sample light comprises two identical pulses separated from each other by 1/(2*f_{shift}).

9. The pulsed UOT system of claim 8, wherein the two identical pulses are Gaussian pulses.

10. The pulsed UOT system of claim 8, wherein the two identical pulses are arbitrarily-shaped pulses.

11. The pulsed UOT system of claim 1, wherein the combined duration of the at least one pulse of the sample light is equal to or less than 500 ns.

12. The pulsed UOT system of claim 1, wherein the combined duration of the at least one pulse of the sample light is equal to or less than 200 ns.

13. The pulsed UOT system of claim 1, wherein the combined duration of the at least one pulse of the sample light is within the range of 100 ns to 1000 ns.

14. The pulsed UOT system of claim 1, wherein the target voxel comprises brain matter.

15. The pulsed UOT system of claim 14, wherein the processor is configured for determining neural activity within the target voxel based on the determined physiologically-dependent optical parameter.

16. The pulsed UOT system of claim 1, wherein each of the at least one interference light pattern comprises a speckle light pattern.

17. The pulsed UOT system of claim 1, wherein the at least one array of detectors is configured for detecting the spatial components of the at least one different interference light pattern, and storing the plurality of values for all of the at least one interference pattern, within 10 milliseconds.

18. The pulsed UOT system of claim 1, wherein the at least one array of detectors is configured for detecting the spatial components of the at least one different interference light pattern, and storing the plurality of values for all of the at least one interference pattern, within 1 millisecond.

19. The pulsed UOT system of claim 1, wherein the target voxel is less than one mm³.

20. The pulsed UOT system of claim 1, wherein the interferometer comprises a light source configured for generating source light, a beam splitter configured for splitting the source light into the sample light and the reference light.

21. The pulsed UOT system of claim 1, wherein the processor is configured for computing the amplitude of the signal light using the plurality of values generated by each detector array, and determining the physiologically-dependent optical parameter of the target voxel based on the computed amplitude of the signal light.

22. The pulsed UOT system of claim 21, wherein the plurality of values generated by each detector array are intensities of the spatial components of the respective interference light pattern, the processor is configured for using the plurality of values generated by each detector array to determine a product of the amplitude of the signal light and a known amplitude of the reference light, and determining the amplitude of the signal light from the determined product.

23. A method of performing pulsed ultrasound modulated optical tomography (UOT), comprising:
delivering ultrasound into a target voxel within an anatomical structure;
delivering sample light into the anatomical structure, whereby a portion of the sample light passing through the target voxel is scattered by the anatomical structure as signal light, and another portion of the sample light not passing through the target voxel is scattered by the anatomical structure as background light that combines with the signal light to create a sample light pattern;
pulsing the ultrasound and the sample light in synchrony at the target voxel, such that only the signal light is frequency shifted by the ultrasound at the target voxel, such that at least one pulse of the sample light has a combined duration less than a pulse width of the ultrasound;
combining reference light with the sample light pattern to generate at least one interference light pattern, such that at least one component of the signal light and the reference light are combined in a heterodyne manner;
detecting spatial components of each of the at least one interference light pattern;
storing a plurality of values representative of the respective spatial components of each of the at least one interference light pattern; and
determining a physiologically-dependent optical parameter of the target voxel based on the plurality of values.

24. The method of claim 23, wherein the at least one pulse of the sample light comprises only a single pulse.

25. The method of claim 23, wherein the at least one pulse of the sample light comprises a plurality of pulses.

26. The method of claim 23, further configured shifting the sample light relative to the reference light by a frequency different from the frequency of the ultrasound, such that the signal light and the reference light are combined in the heterodyne manner.

27. The method of claim 26, wherein
$${\int }_{0}^{1 / {f}_{shift}} \sqrt{{P}_{reference} ( t ) * {P}_{background} ( t )} \times sin ( 2 π {f}_{shift} + {α}_{unknown} ) dt \approx 0 ,$$
wherein P_{reference}(t) is the time varying component of the reference light as a function of time t, P_{background}(t) is the time varying component of the background light as a function of time t, and f_{shift} equals the frequency at which the sample light is shifted relative to the reference light by the interferometer.

28. The method of claim 27, wherein at least 99% of the P_{background}(t) is eliminated from the interference light pattern.

29. The method of claim 27, wherein the at least one pulse of the sample light is a single rectangular pulse, and wherein f_{shift}*τₒₚ=1, wherein τₒₚ is the width of the single rectangular pulse.

30. The method of claim 27, wherein the at least one pulse of the sample light comprises two identical pulses separated from each other by 1/(2*f_{shift}).

31. The method of claim 30, wherein the two identical pulses are Gaussian pulses.

32. The method of claim 30, wherein the two identical pulses are arbitrarily-shaped pulses.

33. The method of claim 23, wherein the combined duration of the at least one pulse of the sample light is equal to or less than 500 ns.

34. The pulsed UOT system of claim 23, wherein the combined duration of the at least one pulse of the sample light is equal to or less than 200 ns.

35. The method of claim 23, wherein the combined duration of the at least one pulse of the sample light is within the range of 100 ns to 1000 ns.

36. The method of claim 23, wherein the target voxel comprises brain matter.

37. The method of claim 36, further comprising determining neural activity within the target voxel based on the determined physiologically-dependent optical parameter.

38. The method of claim 23, wherein each of the at least one interference light pattern comprises a speckle light pattern.

39. The method of claim 23, the spatial components of the at least one different interference light pattern are detected, and the plurality of values for all of the at least one interference pattern are stored, within 10 milliseconds.

40. The method of claim 23, the spatial components of the at least one different interference light pattern are detected, and the plurality of values for all of the at least one interference pattern are stored, within 1 millisecond.

41. The method of claim 23, wherein the target voxel is less than one mm³.

42. The method of claim 23, further comprising generating source light, and splitting the source light into the sample light and the reference light.

43. The method of claim 23, further comprising computing the amplitude of the signal light using the plurality of values, wherein the physiologically-dependent optical parameter of the target voxel is determined based on the computed amplitude of the signal light.

44. The method of claim 43, wherein the plurality of values are intensities of the spatial components of the respective interference light pattern, wherein the plurality of values are used to determine a product of the amplitude of the signal light and a known amplitude of the reference light, and the amplitude of the signal light is determined from the determined product.

45. A pulsed ultrasound modulated optical tomography (UOT) system, comprising:
an interferometer configured for delivering sample light into an anatomical structure having a target voxel, whereby a portion of the sample light passing through the target voxel is scattered by the anatomical structure as signal light, and another portion of the sample light not passing through the target voxel is scattered by the anatomical structure as background light that is combined with the signal light to create a sample light pattern, the interferometer further configured for shifting the sample light relative to the reference light by a frequency different from the frequency of the ultrasound, and combining reference light with the sample light pattern to generate at least one interference light pattern, such that the signal light and the reference light are combined in a heterodyne manner.
an acoustic assembly configured for delivering ultrasound into the target voxel, such that the signal light is frequency shifted by the ultrasound;
a controller configured for operating the acoustic assembly and the interferometer to pulse the ultrasound and the sample light in synchrony at the target voxel, such that at least one pulse of the sample light has a combined duration different from a pulse width of the ultrasound, wherein
$${\int }_{0}^{1 / {f}_{shift}} \sqrt{{P}_{reference} ( t ) * {P}_{background} ( t )} \times sin ( 2 π {f}_{shift} + {α}_{unknown} ) dt \approx 0 ,$$
wherein P_{reference}(t) is the time varying component of the reference light as a function of time t, P_{background}(t) is the time varying component of the background light as a function of time t, and f_{shift} equals the frequency at which the sample light is shifted relative to the reference light by the interferometer;
at least one array of detectors configured for detecting spatial components of each of the at least one interference light pattern and storing a plurality of values representative of the respective spatial components of each of the at least one interference light pattern; and
a processor configured for determining a physiologically-dependent optical parameter of the target voxel based on the plurality of values.
